SEQUENCE LISTING (1 sequence)

SEQ ID NO: 1 - DNA, 24 bp, synthetic construct
  source 1..24: mol_type = other DNA
ttagggttagggttagggttaggg

(19) United States
(12) Patent Application Publication
(10) Pub. No.: US 2025/0313862 A1
(43) Pub. Date: Oct. 9, 2025

(54) INHIBITION OF GENOTOXIC STRESS TO IMPROVE T CELL ENGINEERING

(71) Applicant: GENENTECH, INC., South San Francisco, CA (US)

(72) Inventors: Shuya WANG, South San Francisco, CA (US); Christian Anh-Tu HUYNH, South San Francisco, CA (US); David SHAW, South San Francisco, CA (US); Shahram MISAGHI, South San Francisco, CA (US)

(21) Appl. No.: 19/090,269

(22) Filed: Mar. 25, 2025

Related U.S. Application Data
(63) Continuation of application No. PCT/US2023/075156, filed on Sep. 26, 2023.
(60) Provisional application No. 63/377,251, filed on Sep. 27, 2022.

(51) Int. Cl. C12N 15/90; C07K 14/725; C12N 5/0783; C12N 9/22; C12N 15/11

(52) U.S. Cl. CPC C12N 15/907; C07K 14/7051; C12N 5/0636; C12N 9/226; C12N 15/11; C12N 2310/20; C12N 2501/24; C12N 2501/727; C12N 2510/00

(57) ABSTRACT

Provided herein, inter alia, are methods and compositions for engineering T cells. The methods include contacting a T cell with a nucleic acid and one or more cyclic GMP-AMP synthase (cGAS)-stimulator of interferon gene (STING) pathway inhibitors. The methods provided herein are contemplated to increase cell viability, expansion and gene editing efficiency, thereby allowing an increase in the total number of engineered T cells.

### CROSS-REFERENCE TO RELATED APPLICATIONS

This application claims priority to U.S. Provisional Application No. 63/377,251, filed Sep. 27, 2022, which is hereby incorporated by reference in its entirety and for all purposes.

### REFERENCE TO AN ELECTRONIC SEQUENCE LISTING

The contents of the electronic sequence listing (048893-565001WO_Sequence_Listing_ST26.xml; Size: 1,720 bytes; and Date of Creation: Sep. 26, 2023) is hereby incorporated by reference in its entirety.

### BACKGROUND

Gene editing allows for engineering of various cultured cell lines and primary cells, including T cells. Editing of cell genomes enables generation of T cells that are specific for disease targets, for example cancer cell antigens recognized by edited T cell receptors, allowing for production of personalized cancer therapeutics. However, methods for engineering T cells often result in low recovery rates of the final cell product. For example, the toxic impact of T cell engineering methods can lead to decreases in cell viability and expansion rates. Moreover, previous methods of engineering T cells often resulted in low gene editing efficiency, further contributing to low total engineered T cell number.

Disclosed herein, inter alia, are solutions to these and other problems in the art.

### BRIEF SUMMARY

In an aspect is provided a method of engineering a T cell, including contacting the T cell with a nucleic acid and one or more cyclic GMP-AMP synthase (cGAS)-stimulator of interferon gene (STING) pathway inhibitors.

In an aspect is provided a method of increasing cell viability of a population of engineered T cells, including contacting a population of T cells with one or more cyclic GMP-AMP synthase (cGAS)-stimulator of interferon gene (STING) pathway inhibitors and a nucleic acid, thereby forming the population of engineered T cells. In embodiments, the population of engineered T cells has increased cell viability relative to a population of engineered T cells wherein the population of T cells are not contacted with one or more cGAS-STING pathway inhibitors.

In an aspect is provided a method of increasing gene editing efficiency in a population of T cells, including contacting the population of T cells with one or more cyclic GMP-AMP synthase (cGAS)-stimulator of interferon gene (STING) pathway inhibitors and a nucleic acid, thereby forming a population of engineered T cells. In embodiments, the population of T cells has increased gene editing efficiency relative to a population of T cells that are not contacted with one or more cGAS-STING pathway inhibitors.

In an aspect is provided a method for increasing expansion of a population of engineered T cells, including i) contacting a population of T cells with one or more cyclic GMP-AMP synthase (cGAS)-stimulator of interferon gene (STING) pathway inhibitors and a nucleic acid, thereby forming the population of engineered T cells, and ii) expanding the population of engineered T cells, thereby forming a population of expanded engineered T cells. In embodiments, the one or more cGAS-STING pathway inhibitors increases the population of expanded engineered T cells relative to a population of engineered T cells, wherein the population of T cells of step i) are not contacted with one or more cGAS-STING pathway inhibitors.

In an aspect is provided an engineered T cell, made by a method provided herein including embodiments thereof.

In an aspect is provided a population of engineered T cells made by contacting a population of T cells with a nucleic acid and one or more cyclic GMP-AMP synthase (cGAS)-stimulator of interferon gene (STING) pathway inhibitors.

In an aspect is provided a composition including a population of T cells, a nucleic acid, and one or more cyclic GMP-AMP synthase (cGAS)-stimulator of interferon gene (STING) pathway inhibitors.

In an aspect is provided a pharmaceutical composition including an engineered T cell provided herein including embodiments thereof.

In an aspect is provided a method of treating a disease in a subject in need thereof, including administering a therapeutically effective amount of an engineered T cell provided herein including embodiments thereof or a pharmaceutical composition provided herein including embodiments thereof.

### BRIEF DESCRIPTION OF THE DRAWINGS

FIGS. 1A-1F. Plasmid DNA mediated activation of cGAS-STING pathway negatively affects T cell viability and expansion post electroporation. (FIG. 1A) Schematic of exogenic T cell receptor DNA templates used for CRISPR/Cas9 mediated homology directed repair at the TCR-α locus. DNA template including TCR-α variant chain and TCR-β chain is designed to insert into TCR-α (TRAC) locus while endogenous TCR-α (VJ) and TCR-β have been disrupted; (FIG. 1B) Schematic of 15-day work flow of T cell activation, engineering and cell culture process. The whole process is performed in chemically defined media; (FIGS. 1C-1E) Analysis of T cell viability (FIG. 1C), T cell expansion (FIG. 1D) and knock-in/out efficiency (FIG. 1E) during a 15-day T cell engineer and culture process; T cell viability and cell expansion were measured by NucleoCounter NC-200. Knock-in/out efficiency was measured by flow cytometry and identified by MHC-peptide dextramer staining using flow cytometry. (FIG. 1F) Western blot analysis of DNA genotoxic pathway activation (cGAS-STING-TBK1-IRF3) during and after TCR engineering process in presence of different components of transfection used for gene editing, as indicated (RNP, DNA).

FIGS. 2A-2C. T cell viability, expansion, and targeting data for a different donor 48 h-post transfection. T cell viability (FIG. 2A), T cell expansion rate (FIG. 2B) and knock-in/out efficiency (FIG. 2C) with different transfection process components as indicated (DNA, RNP). T cell viability data and cell expansion rate were measured by NucleoCounter NC-200. Knock-in/out efficiency was measured by flow cytometry and identified by MHC-peptide dextramer staining in flow cytometry.

FIGS. 3A-3B. Western blot analysis of AIM2 DNA inflammasome and toll-like-receptor (TLRs) pathway activation during and after TCR engineering process in the presence of different transfection components, as indicated (RNP, DNA). (FIG. 3A) Cell lysates collected during the T cell engineering process were analyzed for activation of Aim2-Cas1-IL-1B inflammasome pathway. (FIG. 3B) Cell lysates collected during the T cell engineering process were analyzed for activation of MyD88-TRAF6 TLRs pathway.

FIGS. 4A-4I. Individual donor T cell response data after inhibitor treatment (three donors). (FIGS. 4A, 4D, 4G) Percent viability 48 h post transfection was determined using NucleoCounter NC-200 for donors 1, 2 and 3, respectively; (FIGS. 4B, 4E, 4H) final drug product (FDP) expansion folds after a 15-day T cell engineering process corresponding to donors 1, 2, and 3, respectively, calculated based on seeding cell number on day 2; (FIGS. 4C, 4F, 4I) total edited cell (TEC) number in FDP corresponding to donors 1, 2, and 3, respectively. The TEC were calculated based on 2 million cell seeding density post transfection per group. Numbers on each column are rounded up to whole integers. Total edited cell number was calculated by multiplying the expansion fold with the knock-in percentage.

FIGS. 5A-5F: Individual donor T cell response data after inhibitor treatment (two more donors in addition to that of FIG. 4). (FIGS. 5A, 5D) Percent viability 48 h post transfection was determined using NucleoCounter NC-200 for donors 4 and 5, respectively; (FIGS. 5B, 5E) final drug product (FDP) expansion folds after a 15-day T cell engineering process for donors 4 and 5, respectively, calculated based on seeding cell number on day 2, (FIGS. 5C, 5F) Total edited cell (TEC) number in FDP for donors 4 and 5, respectively. The TEC were calculated based on 2 million cell seeding density post transfection per group. Numbers on each column are rounded up to whole integers. Total edited cell number was calculated by multiplying the expansion fold with the knock-in percentage.

FIGS. 6A-6C: Summary data of 48 h-post transfection percent viability (FIG. 6A), final cell product expansion fold (FIG. 6B) and total edited cell (TEC) number (FIG. 6C) from five independent donors. T cell viability data and expansion folds were measured by the NucleoCounter NC-200 machine. TEC was calculated by expansion fold multiplied by the knock-in percentage. Data presented as mean±SEM (FIGS. 6A-6C). *** P<0.001; ** P<0.01; *P<0.05 compared with the control (CTR) group.

FIGS. 7A-7C: Final T cell product cell editing ratio. (FIGS. 7A-7C) Knock-in and knock out data from 3 representative donors, respectively, measured by flow cytometry. Knock-in ratio was identified by MHC-peptide dextramer and TCR double positive staining in flow cytometry. Knock-out is representative of MHC-peptide dextramer and TCR double negative. Control (CTR) represents no inhibitor treatment group.

FIGS. 8A-8F. BX795 pretreatment attenuates T cell cGAS-STING pathway. (FIG. 8A) Western blot analysis of TBK1 and IRF3 expression and phosphorylation with or without (CTR group) BX795 pretreatment. N, 30 min and 60 min represent immediately prior to transfection, 30 min post transfection and 60 min post transfection, respectively. (FIGS. 8B-8E) Percent viability, expansion fold, knock-in/out efficiency, and total edited cell numbers obtained from the same donor as in (FIG. 8A). Viability data (FIG. 8B) was collected 48 h post transfection by NucleoCounter NC-200. Expansion fold (FIG. 8C), knock-in/out efficiency (FIG. 8D) and total edited cells (FIG. 8E) values represent data obtained from the last day of culture (day 15). (FIG. 8F) Cytokine expression comparison (using qPCR) of groups with and without inhibitor treatment. Samples used for qPCR analysis were collected 6 h post transfection and analyzed in triplicates.

FIGS. 9A-9D: Inhibitor pretreatment improves knock-out and knock-in ratio in the final T cell product. (FIG. 9A) Total knock-out percentage of BX795 pre-treatment groups, compared with the control groups. (FIG. 9B) Analysis of the percentage difference of knock-out values between control, which is set at 1, and inhibitor pretreatment groups. (FIG. 9C) Knock-in ratio of BX795 pre-treatment groups compared with the control group. (FIG. 9D) Analysis of the percentage difference in knock-in among BX795 pretreatment groups and control group, which is set at 1. Samples were collected on day 15 and ratios were measured by flow cytometry. Knock-in/out ratio was identified by MHC-peptide dextramer staining in flow cytometry. *** P<0.001; ** P<0.01; * P<0.05 compared with the control group.

FIGS. 10A-10F: Pretreatment of T cell cultures with BX795 prior to transfection had no impact on the final T cell product phenotype. T cell phenotype data shown in FIGS. 10A-10F were collected from six independent donors, respectively. Inhibitor type and dosage were used as indicated. Cell samples were collected on day 15 and phenotype ratio was measured by flow cytometry. T stem cell memory (TSCM: CD45RA⁺CD45RO⁻CD95⁺CD27⁺), T central memory (TCM: CD45RA⁺CD45RO⁺CD95⁺CD27⁺), T cell effector memory (TEM: CD45RA⁺CD45RO⁺CD95⁺CD27⁻), and effector T cells (TE:CD45RA⁺CD45RO⁻CD95⁺CD27⁻) phenotypes were measured as indicated. All six donors were treated similarly with regards to inhibitor pretreatment, electroporation process, and culture conditions and methods.

FIGS. 11A-11F. T cell cultures pretreated with BX795 inhibitor prior to transfection displayed similar functional potential to that of untreated control groups. (FIG. 11A) Activation marker CD137 expression comparison between inhibitor pretreated and control groups. Cytokine expression: IFNγ (FIG. 11B), TNF-α (FIG. 11C), and Granzyme B (FIG. 11D), comparison between inhibitor pretreated and control groups. Cell samples were collected on the final day (day 15). Cells were then co-cultured with target peptide (WT1) at indicated concentration for 24 h prior to flow cytometry analysis. (FIG. 11E) Proliferation analysis comparison between inhibitor pretreated and control groups. Cell samples were collected on the final day (day 15). Cells were then labeled with CFSE dye and co-cultured with target peptide (WT1) at the indicated concentrations for 72 h prior to flow cytometry analysis. (FIG. 11F) Cell killing analysis to compare T cell function between inhibitor pretreated and control groups. Cell samples were collected on the final day (day 15) and then co-culture with target peptide pre-labeled T2 target cells for 20 h at the indicated ratios. Target cell apoptosis levels were measured by calculating annexin V and 7-aminoactinomycin D (7-AAD) double positive populations using flow cytometry, corrected by the target cell-only group.

FIGS. 12A-12B. Inhibitor pretreated and control groups have comparable T cell exhaustion profiles. Median fluorescence intensity (MFI) of Tim3 (FIG. 12A) and PD-1 (FIG. 12B) were analyzed by flow cytometry. Cell samples were collected on the final day (day 15) of the process. Cells were then co-cultured with target peptide (WT1) at the indicated concentration for 24 h prior to flow cytometry analysis.

FIGS. 13A-13C. T cell cultures pretreated with BX795 inhibitor prior to transfection displayed similar functional potential to that of untreated control groups in three different donors. More donors (donors 1-3) were analyzed for T cell function, respectively similar as shown in FIGS. 11A-11F. Activation marker CD137, cytokine expression of IFNγ, TNF-α, GranzymeB and Perforin expression for inhibitor pretreated and control groups were measured. Cell samples were collected on the final day (day 15) of the process. Cells were then co-cultured with target peptide (WT1) at indicated concentration for 24 h before analysis by flow cytometry. For proliferation analysis comparison between inhibitor pretreated and control groups, samples were collected on day 15 and cells were then labeled by CFSE dye and co-cultured with target peptide (WT1) at indicated concentration for 72 h prior to flow cytometry analysis. For cell killing analysis, T cell function between inhibitor pretreated and control groups were compared. Cell samples were collected on day 15 and were co-cultured with target peptide pre-labeled T2 target cells for 20 h at the indicated ratios. Target cell apoptosis levels were measured by calculating annexin V and 7-aminoactinomycin D (7-AAD) double positive populations using flow cytometry, corrected by the target cell-only group.

FIGS. 14A-14C. Plasmid DNA transfection efficiency depends on electroporation (pulse code) program used. Inhibitor BX795 enhances cell editing efficiency (knock-in) throughout the 15-day culture process. (FIG. 14A) GFP positive CD8+ T cells percentage comparison between different electroporation pulse codes used. EH115 (strong), EW113 (medium), EW100 (mild). 1 ug/ml GFP plasmid was used to analyze DNA uptake based on vendor's instruction. GFP positive CD8+ T cells percentage is measured by flow cytometry 24 h post electroporation. EH115 offers the harshest condition of the three pulse-codes, while EW100 is recommended by the vendor as the basic pulse code with the mildest transfection condition. (FIGS. 14B-C) T cell knock-in and knock out data from untreated control group (FIG. 14B) and BX795 (2.5 uM) pre-treated group (FIG. 14C), according to date, measured by flow cytometry. Transfection was done by day 2 and measurement starts from day 4 (48 h post transfection). Knock-in ratio was identified by double positive of MHC-peptide dextramer staining and TCR staining by flow cytometry. Knock-out is representative of MHC-peptide dextramer and TCR double negative population.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g., mRNA, siRNA, miRNA, and guide RNA and any types of DNA, e.g., genomic DNA, plasmid DNA, minicircle DNA, linear DNA, and any fragments thereof.

As used herein, the term “gene editing reagent” refers to components required for gene editing tools and may include enzymes, riboproteins, solutions, co-factors and the like. For example, gene editing reagents include one or more components required for Zinc finger nucleases (ZFNs), transcription activator like effector nucleases (TALEN), meganucleases, and clustered regularly interspaced short palindromic repeats system (CRISPR/Cas) gene editing.

As used herein, “zinc finger protein” (ZFP) refers to a chimeric protein comprising a nuclease domain and a nucleic acid (e.g., DNA) binding domain that is stabilized by zinc. The individual DNA binding domains are typically referred to as “fingers,” such that a zinc finger protein or polypeptide has at least one finger, more typically two fingers, or three fingers, or even four or five fingers, to at least six or more fingers. Each finger typically binds from two to four base pairs of DNA. Each finger may comprise about 30 amino acids zinc-chelating, DNA-binding region (see, e.g., U.S. Pat. Publ. No. 2012/0329067 A1, the disclosure of which is incorporated herein by reference).

As used herein, “transcription activator-like effectors” (TALEs) refer to proteins composed of more than one TAL repeat and is capable of binding to nucleic acid in a sequence specific manner. TALEs represent a class of DNA binding proteins secreted by plant-pathogenic bacteria of the species, such as Xanthomonas and Ralstonia, via their type III secretion system upon infection of plant cells. Natural TALEs specifically have been shown to bind to plant promoter sequences thereby modulating gene expression and activating effector-specific host genes to facilitate bacterial propagation (Römer, P., et al., Science 318:645-648 (2007); Boch, J., et al., Annu. Rev. Phytopathol. 48:419-436 (2010); Kay, S., et al., Science 318:648-651 (2007); Kay, S., et al., Curr. Opin. Microbiol. 12:37-43 (2009)). The modular structure of TALs allows for combination of the DNA binding domain with effector molecules such as nucleases. In particular, TALE nucleases allow for the development of new genome engineering tools.

Natural TALEs are generally characterized by a central repeat domain and a carboxyl-terminal nuclear localization signal sequence (NLS) and a transcriptional activation domain (AD). The central repeat domain typically consists of a variable amount of between 1.5 and 33.5 amino acid repeats that are usually 33-35 residues in length except for a generally shorter carboxyl-terminal repeat referred to as half-repeat. The repeats are mostly identical but differ in certain hypervariable residues. DNA recognition specificity of TALEs is mediated by hypervariable residues typically at positions 12 and 13 of each repeat—the so-called repeat variable diresidue (RVD) wherein each RVD targets a specific nucleotide in a given DNA sequence. Thus, the sequential order of repeats in a TAL protein tends to correlate with a defined linear order of nucleotides in a given DNA sequence. The underlying RVD code of some naturally occurring TALEs has been identified, allowing prediction of the sequential repeat order required to bind to a given DNA sequence (Boch, J., et al., Science 326:1509-1512 (2009); Moscou, M. J., et al., Science 326:1501 (2009)). Further, TAL effectors generated with new repeat combinations have been shown to bind to target sequences predicted by this code. It has been shown that the target DNA sequence generally start with a 5′ thymine base to be recognized by the TAL protein.

The term “RNA-guided DNA nuclease” or “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system. The C2c1 (“Class 2 candidate 1”) enzyme is a Class II type V-B enzyme. The C2c2 (“Class 2 candidate 2”) enzyme is a Class II type VI-A enzyme. The C2c3 (“Class 2 candidate 3”) enzyme is a Class II type V-C enzyme. Non-limiting exemplary CRISPR associated proteins include Cas1, Cas1B, Cas2, Cas3, Cas4, Cas5, Cas6, Cas7, Cas8, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

A “CRISPR-associated endonuclease Cas12a,” “Cas12a,” “Cas12” or “Cas 12 protein” as referred to herein includes any of the recombinant or naturally-occurring forms of the Cas12 endonuclease or variants or homologs thereof that maintain Cas12 endonuclease enzyme activity (e.g. within at least 50%, 80%, 90%, 95%, 96%, 97%, 98%, 99% or 100% activity compared to Cas12). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas12 protein. In aspects, the Cas12 protein is substantially identical to the protein identified by the UniProt reference number AOQ7Q2 or a variant or homolog having substantial identity thereto.

A “Cfp1” or “Cfp1 protein” as referred to herein includes any of the recombinant or naturally-occurring forms of the Cfp1 (CxxC finger protein 1) endonuclease or variants or homologs thereof that maintain Cfp1 endonuclease enzyme activity (e.g. within at least 50%, 80%, 90%, 95%, 96%, 97%, 98%, 99% or 100% activity compared to Cfp1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cfp1 protein. In embodiments, the Cfp1 protein is substantially identical to the protein identified by the UniProt reference number Q9P0U4 or a variant or homolog having substantial identity thereto.

The term “RNA-guided RNA nuclease” or “RNA-guided RNase” and the like refer, in the usual and customary sense, to an RNA-guided nuclease that targets a specific phosphodiester bond within an RNA polynucleotide, wherein the recognition of the phosphodiester bond is facilitated by a separate polynucleotide sequence (for example, a RNA sequence (e.g., single guide RNA (sgRNA), a guide RNA (gRNA)). Typically, an RNA guided RNase targets single-stranded RNA. In aspects, the RNA-guided RNase is Cas13 (e.g. Cas13a, Cas13b).

A “Cas13a” or “Cas13a protein” as referred to herein includes any of the recombinant or naturally-occurring forms of the Cas13a (CRISPR-associated endoribonuclease Cas13a) endonuclease, also known as CRISPR-associated endoribonuclease C2c2, C2c2, or variants or homologs thereof that maintain Cas13a endonuclease enzyme activity (e.g. within at least 50%, 80%, 90%, 95%, 96%, 97%, 98%, 99% or 100% activity compared to Cas13a). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13a protein. In embodiments, the Cas13a protein is substantially identical to the protein identified by the UniProt reference number C7NBY4 or a variant or homolog having substantial identity thereto.

A “Cas13b” or “Cas13b protein” as referred to herein includes any of the recombinant or naturally-occurring forms of the Cas13b (CRISPR-associated RNA-guided ribonuclease Cas13b) endonuclease, or variants or homologs thereof that maintain Cas13b nuclease enzyme activity (e.g. within at least 50%, 80%, 90%, 95%, 96%, 97%, 98%, 99% or 100% activity compared to Cas13b). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13b protein. In embodiments, the Cas13b protein is substantially identical to the protein identified by the UniProt reference number A0A8G0P913 or a variant or homolog having substantial identity thereto.

In embodiments, the gene editing reagent comprises Cas-CLOVER. In embodiments, Cas-CLOVER comprises Clo051 nuclease domain fused with catalytically dead Cas9. See, e.g., U.S. Patent Pub. No. US2021/0107993, and Madison et al., Molecular Therapy Nucleic Acids, Vol. 29, P979-995, Sep. 13, 2022, each of which is incorporated by reference herein in its entirety. In embodiments, the gene editing reagent comprises a nickase, e.g., nCas9 (nickase Cas9). Nickases are engineered Cas proteins capable of introducing a single-strand cut with the same specificity as a regular CRISPR/Cas nuclease. See, e.g., PCT Pub. No. WO2014093694, which is incorporated herein by reference in its entirety.

The terms “guide RNA” and “gRNA” are used interchangeably and refer to the polynucleotide sequence including the crRNA sequence and optionally the tracrRNA sequence. In embodiments, the gRNA includes the crRNA sequence and the tracrRNA sequence (e.g., “single guide RNA” or “sgRNA”). In embodiments, the gRNA does not include the tracrRNA sequence. The crRNA sequence includes a guide sequence (i.e., “guide” or “spacer”) and a tracr mate sequence (i.e., direct repeat(s)). The term “guide sequence” refers to the sequence that specifies the target site. In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) comprises the tracr mate sequence hybridized to the tracr sequence.

In embodiments, the gRNA is a single-stranded ribonucleic acid. In aspects, the gRNA is from about 10 to about 200 nucleic acid residues in length. In aspects, the gRNA is from about 50 to about 150 nucleic acid residues in length. In aspects, the gRNA is from about 80 to about 140 nucleic acid residues in length. In aspects, the gRNA is from about 90 to about 130 nucleic acid residues in length. In aspects, the gRNA is from about 100 to about 120 nucleic acid residues in length.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

As used herein, the term “donor DNA” refers to a single-stranded or double-stranded DNA that can be inserted into the genome of a cell (e.g. a T cell) using genetic modification methods (e.g. CRISPR). For example, the donor DNA may have homology arms that are homologous to a region of a gene where the donor DNA is to be inserted. For example, the donor DNA may form a complex with a Cas protein. In instances, the cell may be transfected with gene editing reagents and the donor DNA. In embodiments, the donor DNA is part of a plasmid, vector, or expression vector that facilitates delivery of the donor DNA into a cell. In embodiments, the donor DNA is part of a circular DNA. In embodiments, the donor DNA is part of a linear DNA. In embodiments, the donor DNA may include one or more modifications.

Nucleic acids (such as donor DNA) used in the methods herein may be modified. For example, the nucleic acids may include known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds.

Nucleic acids containing one or more carbocyclic sugars are also included within one definition of nucleic acids. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

A “TANK-binding kinase 1 protein” or “TBK1” as used herein includes any of the recombinant or naturally-occurring forms of TANK-binding kinase 1 (TBK1), also known as Serine/threonine-protein kinase TBK1, NF-kappa-B-activating kinase, T2K or variants or homologs thereof that maintain TANK-binding kinase 1 (TBK1) activity (e.g. within at least 50%, 80%, 90%, 95%, 96%, 97%, 98%, 99% or 100% activity compared to TBK1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TBK1 protein. In aspects, the TBK1 protein is substantially identical to the protein identified by the UniProt reference number Q9UHD2 or a variant or homolog having substantial identity thereto.

A “Cyclic GMP-AMP synthase protein” or “cGAS” as used herein includes any of the recombinant or naturally-occurring forms of Cyclic GMP-AMP synthase protein (cGAS) or variants or homologs thereof that maintain cGAS activity (e.g. within at least 50%, 80%, 90%, 95%, 96%, 97%, 98%, 99% or 100% activity compared to cGAS). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cGAS protein. In aspects, the cGAS protein is substantially identical to the protein identified by the UniProt reference number Q8N884 or a variant or homolog having substantial identity thereto.

A “Stimulator of interferon genes protein” or “STING” as used herein includes any of the recombinant or naturally-occurring forms of Stimulator of interferon genes protein (STING), also referred to as Endoplasmic reticulum interferon stimulator, Mediator of IRF3 activation, Transmembrane protein 173 or variants or homologs thereof that maintain STING activity (e.g. within at least 50%, 80%, 90%, 95%, 96%, 97%, 98%, 99% or 100% activity compared to STING). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring STING protein. In aspects, the STING protein is substantially identical to the protein identified by the UniProt reference number Q86WV6 or a variant or homolog having substantial identity thereto.

A “Interferon regulatory factor 3 protein” or “IRF3” as used herein includes any of the recombinant or naturally-occurring forms of Interferon regulatory factor 3 (IRF3) or variants or homologs thereof that maintain IRF3 activity (e.g. within at least 50%, 80%, 90%, 95%, 96%, 97%, 98%, 99% or 100% activity compared to IRF3). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IRF3 protein. In aspects, the IRF3 protein is substantially identical to the protein identified by the UniProt reference number Q14653 or a variant or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. In embodiments, the plasmid, vector, or expression vector is a circular nucleic acid. In embodiments, the plasmid, vector, or expression vector is not a linear nucleic acid. In embodiments, the plasmid, vector, or expression vector is a linear nucleic acid.

As used herein, the term “nanoplasmid” is used to refer to an circular nucleic acid containing at minimum a nucleic acid(s) sequence of interest, an miniature origin of replication (e.g. R6K), and an selectable marker (e.g. a small RNA selectable marker, RNA-OUT). In embodiments, a nanoplasmid contains less than 500 bp of prokaryotic DNA.

As used herein, the term “minicircle” refers to a circular nucleic acid, generally from about 200 bases to about 5 kilobases in length. In embodiments, a minicircle is about 2 kilobases to about 5 kilobases in length. In embodiments, a minicircle does not include prokaryotic DNA. Thus, in embodiments, a minicircle includes at a minimum a nucleic acid(s) sequence of interest and elements essential for expression of the nucleic acid sequence.

As used herein, the terms “T cell engineering” or “T cell gene engineering” or the like refer to a type of genetic modification in which DNA is inserted, deleted, modified or replaced at one or more specified locations in the genome of a T cell. Unlike early genetic engineering techniques that randomly insert genetic material into a host genome, T cell engineering targets the genetic modification at site specific locations. Gene editing reagents may be used for T cell engineering to, for example, to generate a double stranded break at a specific point within a gene or genome where DNA is inserted. A gene editing reagent may include, for example a clustered regularly interspaced short palindromic repeats system (CRISPR/Cas), ZFN, or TALEN. Thus, an “engineered T cell” is a T cell wherein DNA is inserted, deleted, modified or replaced at one or more specified locations in the T cell genome.

The term “recombinant” when used with reference, e.g., to a virus, cell, nucleic acid, protein, or vector, indicates that the cell (e.g. T cell), nucleic acid, protein or vector, has been modified by the introduction of a heterologous nucleic acid or protein or the alteration of a native nucleic acid or protein, or that the cell is derived from a cell so modified. In instances, recombinant cells express genes that are not found within the native (non-recombinant) form of the cell or express native genes that are otherwise abnormally expressed, under expressed or not expressed at all. Transgenic cells are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid may be recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A nucleic acid that is the predominant species present in a preparation is substantially purified.

As used herein, the term “electroporation”, “electropermeabilization”, and “electrotransfer” are used in accordance with its plain ordinary meaning and refer to a technique in which an electrical field is applied to cells in order to increase the permeability of the cell membrane, allowing chemicals, drugs, proteins, or nucleic acids, or combinations thereof to be introduced into the cell.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector (e.g. adenovirus vector) may be used in the methods described herein. Examples for viral vectors include, but are not limited to retroviral, adenoviral, lentiviral and adeno-associated viral vectors. In some embodiments, the nucleic acid molecules are introduced into a cell using an adenoviral vector following standard procedures well known in the art. The terms “transfection” or “transduction” also refer to introducing proteins into a cell from the external environment. In embodiments, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Transduce” or “transduction” are used according to their plain ordinary meanings and refer to the process by which one or more foreign nucleic acids (i.e. DNA not naturally found in the cell) are introduced into a cell. Transduction may occur by introduction of a virus or viral vector (e.g. adenovirus vector) into the cell.

The word “expression” or “expressed” as used herein in reference to a gene means the transcriptional and/or translational product of that gene (e.g. a TCR-alpha, TCR-beta, etc.). The level of expression of a DNA molecule in a cell may be determined on the basis of either the amount of corresponding mRNA that is present within the cell or the amount of protein encoded by that DNA produced by the cell. The level of expression of nucleic acid molecules may be detected by standard methods, including PCR or Northern blot methods well known in the art. See, Sambrook et al., 1989 Molecular Cloning: A Laboratory Manual, 18.1-18.88.

“Contacting” is used in accordance with its plain ordinary meaning and refers to the process of allowing at least two distinct species (e.g. chemical compounds including biomolecules or cells) to become sufficiently proximal to react, interact or physically touch. The two species may be, for example, a cGAS-STING pathway inhibitor as provided herein and a T cell. In embodiments contacting includes, for example, allowing a cGAS-STING pathway inhibitor described herein to physically touch a T cell. In embodiments, the contacting may result in delivery of a compound into a cell. For example, the contacting may result in delivery of a cGAS-STING inhibitor into a cell. In embodiments, the contacting may result in delivery of a nucleic acid into the cell. In embodiments, “contacting” or “contacted” includes culturing T cells in the presence of a species, e.g., a cGAS-STING pathway inhibitor.

A “control” or “standard control” refers to a sample, measurement, or value that serves as a reference, usually a known reference, for comparison to a test sample, measurement, or value. For example, a standard control may be an engineered T cell made without contacting a T cell with one or more cGAS-STING inhibitors as provided herein including embodiments thereof. In embodiments, a standard control may be a population of engineered T cells made without contacting a population of T cells with one or more cGAS-STING inhibitors as provided herein including embodiments thereof. Thus, the standard control may be an engineered T cell made by contacting a T cell with a nucleic acid without one or more cGAS-STING inhibitors. The standard control may be a population of engineered T cells made by contacting a population of T cells with a nucleic acid without one or more cGAS-STING inhibitors. Controls also are valuable for determining the significance of data. For example, if values for a given parameter are widely variant in controls, variation in test samples will not be considered as significant. One of skill will recognize that standard controls can be designed for assessment of any number of parameters (e.g. cell viability, cell expansion, total edited cell number, gene editing efficiency, etc.).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). In embodiments, “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

The terms “disease” or “condition” refer to a state of being or health status of a patient or subject capable of being treated with the compounds or methods provided herein. The disease may be a cancer. In some further instances, “cancer” refers to human cancers. In embodiments, the cancer is lymphoma, melanoma, or leukemia.

The term “associated” or “associated with” in the context of a substance or substance activity or function associated with a disease (e.g. cancer) is caused by (in whole or in part), or a symptom of the disease is caused by (in whole or in part) the substance or substance activity or function.

“Patient” or “subject in need thereof” refers to a living organism suffering from or prone to a disease (e.g. cancer, etc.) or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form e.g., for injection.

By “therapeutically effective dose or amount” as used herein is meant a dose that produces effects for which it is administered (e.g. treating a disease). The exact dose and formulation will depend on the purpose of the treatment, and will be ascertainable by one skilled in the art using known techniques. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease.

As used herein, the term “administering” is used in accordance with its plain and ordinary meaning and includes any administration appropriate for cell therapy. Parenteral administration includes, e.g., intravenous, intramuscular, intra-arteriole, intradermal, subcutaneous, intraperitoneal, intraventricular, and intracranial. In embodiments, administration is intravenous.

The term “signaling pathway” as used herein refers to a series of interactions between cellular components and optionally extra-cellular components (e.g. proteins, nucleic acids, small molecules, ions, lipids) that conveys a change in one component to one or more other components, which in turn may convey a change to additional components, which is optionally propagated to other signaling pathway components. In embodiments, the signaling pathway is the cyclic GMP-AMP synthase (cGAS)-stimulator of interferon gene (STING) pathway. A signaling pathway may be activated by, for example, the presence of a compound within a cell (e.g. a circular nucleic acid). For example, a signaling pathway may be activated by the presence of a circular nucleic acid (e.g. a plasmid) within the cytoplasm of a cell. In instances, a signaling pathway may be activated by one or more conditions to which the cell is subjected (e.g. transduction, transfection, or electroporation of a nucleic acid into a cell).

### Methods

Provided herein, inter alia, are methods for engineering a T cell, including contacting the T cell with one or more cyclic GMP-AMP synthase (cGAS)-stimulator of interferon gene (STING) pathway inhibitors. The methods provided herein allow formation of an engineered T cell while inhibiting or decreasing cellular toxicity associated with previously known methods of engineering T cells. For example, the methods provided herein are contemplated to be effective for decreasing or inhibiting toxicity associated with the presence of DNA (e.g. plasmid DNA) in the cytoplasm of cells. For example, the methods provided herein are effective for decreasing or inhibiting the cGAS-STING pathway, thereby decreasing or inhibiting cell death. As used herein, “cyclic GMP-AMP synthase-stimulator of interferon genes pathway inhibitor” or “cGAS-STING pathway inhibitor” refers to a compound that inhibits or downregulates the activity or production of any one of the components in the cGAS-STING axis. For example, the cGAS-STING pathway inhibitor may inhibit or downregulate the activity or production of any one of the components of the cGAS pathway. In embodiments, the cGAS-STING pathway inhibitor inhibits binding of cGAS to double-stranded DNA (e.g. plasmid DNA). In embodiments, the cGAS-STING pathway inhibitor inhibits the activity of cGAMP. In another example, the cGAS-STING pathway inhibitor may inhibit or downregulate the activity or production of any one of the components of the STING pathway. In embodiments, the cGAS-STING pathway inhibitor may inhibit STING oligomerization. In another example, the cGAS-STING pathway inhibitor may inhibit or downregulate the activity or production of TANK-binding kinase 1 (TBK1). In embodiments, the cGAS-STING pathway inhibitor inhibits TBK1 phosphorylation or STING phosphorylation. In embodiments, the cGAS-STING pathway inhibitor inhibits phosphorylation of interferon regulatory factor 3 (IRF-3). Thus, in embodiments, the cGAS-STING pathway inhibitor includes a kinase inhibitor (e.g. BX795, Aml, MRT, etc.). In embodiments, the cGAS-STING pathway inhibitor is a kinase inhibitor. In an aspect is provided a method of engineering a T cell, including contacting the T cell with a nucleic acid and one or more cyclic GMP-AMP synthase (cGAS)-stimulator of interferon gene (STING) pathway inhibitors.

In embodiments, the nucleic acid is about 20 bases to about 30000 in length bases in length. In embodiments, the nucleic acid is about 1000 bases to about 30000 in length bases in length. In embodiments, the nucleic acid is about 2000 bases to about 30000 in length bases in length. In embodiments, the nucleic acid is about 3000 bases to about 30000 in length bases in length. In embodiments, the nucleic acid is about 4000 bases to about 30000 bases in length. In embodiments, the nucleic acid is about 5000 bases to about 30000 bases in length. In embodiments, the nucleic acid is about 6000 bases to about 30000 bases in length. In embodiments, the nucleic acid is about 7000 bases to about 30000 bases in length. In embodiments, the nucleic acid is about 8000 bases to about 30000 bases in length. In embodiments, the nucleic acid is about 9000 bases to about 30000 bases in length. In embodiments, the nucleic acid is about 10000 bases to about 30000 bases in length. In embodiments, the nucleic acid is about 11000 bases to about 30000 bases in length. In embodiments, the nucleic acid is about 12000 bases to about 30000 bases in length. In embodiments, the nucleic acid is about 13000 bases to about 30000 bases in length. In embodiments, the nucleic acid is about 14000 bases to about 30000 bases in length. In embodiments, the nucleic acid is about 15000 bases to about 30000 bases in length. In embodiments, the nucleic acid is about 16000 bases to about 30000 bases in length. In embodiments, the nucleic acid is about 17000 bases to about 30000 bases in length. In embodiments, the nucleic acid is about 18000 bases to about 30000 bases in length. In embodiments, the nucleic acid is about 19000 bases to about 30000 bases in length. In embodiments, the nucleic acid is about 20000 bases to about 30000 bases in length. In embodiments, the nucleic acid is about 21000 bases to about 30000 bases in length. In embodiments, the nucleic acid is about 22000 bases to about 30000 bases in length. In embodiments, the nucleic acid is about 23000 bases to about 30000 bases in length. In embodiments, the nucleic acid is about 24000 bases to about 30000 bases in length. In embodiments, the nucleic acid is about 25000 bases to about 30000 bases in length. In embodiments, the nucleic acid is about 26000 bases to about 30000 bases in length. In embodiments, the nucleic acid is about 27000 bases to about 30000 bases in length. In embodiments, the nucleic acid is about 28000 bases to about 30000 bases in length. In embodiments, the nucleic acid is about 29000 bases to about 30000 bases in length.

In embodiments, the nucleic acid is about 20 bases to about 29000 bases in length. In embodiments, the nucleic acid is about 20 bases to about 28000 bases in length. In embodiments, the nucleic acid is about 20 bases to about 27000 bases in length. In embodiments, the nucleic acid is about 20 bases to about 26000 bases in length. In embodiments, the nucleic acid is about 20 bases to about 25000 bases in length. In embodiments, the nucleic acid is about 20 bases to about 24000 bases in length. In embodiments, the nucleic acid is about 20 bases to about 23000 bases in length. In embodiments, the nucleic acid is about 20 bases to about 22000 bases in length. In embodiments, the nucleic acid is about 20 bases to about 21000 bases in length. In embodiments, the nucleic acid is about 20 bases to about 20000 bases in length. In embodiments, the nucleic acid is about 20 bases to about 19000 bases in length. In embodiments, the nucleic acid is about 20 bases to about 18000 bases in length. In embodiments, the nucleic acid is about 20 bases to about 17000 bases in length. In embodiments, the nucleic acid is about 20 bases to about 16000 bases in length. In embodiments, the nucleic acid is about 20 bases to about 15000 bases in length. In embodiments, the nucleic acid is about 20 bases to about 14000 bases in length. In embodiments, the nucleic acid is about 20 bases to about 13000 bases in length. In embodiments, the nucleic acid is about 20 bases to about 12000 bases in length. In embodiments, the nucleic acid is about 20 bases to about 11000 bases in length. In embodiments, the nucleic acid is about 20 bases to about 10000 bases in length. In embodiments, the nucleic acid is about 20 bases to about 9000 bases in length. In embodiments, the nucleic acid is about 20 bases to about 8000 bases in length. In embodiments, the nucleic acid is about 20 bases to about 7000 bases in length. In embodiments, the nucleic acid is about 20 bases to about 6000 bases in length. In embodiments, the nucleic acid is about 20 bases to about 5000 bases in length. In embodiments, the nucleic acid is about 20 bases to about 4000 bases in length. In embodiments, the nucleic acid is about 20 bases to about 3000 bases in length. In embodiments, the nucleic acid is about 20 bases to about 2000 bases in length. In embodiments, the nucleic acid is about 20 bases to about 1000 bases in length. In embodiments, the nucleic acid is about 20, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, or 30000 in length.

In embodiments, the nucleic acid is about 20 bases to about 5000 bases in length. In embodiments, the nucleic acid is about 50 bases to about 5000 bases in length. In embodiments, the nucleic acid is about 100 bases to about 5000 bases in length. In embodiments, the nucleic acid is about 150 bases to about 5000 bases in length. In embodiments, the nucleic acid is about 200 bases to about 5000 bases in length. In embodiments, the nucleic acid is about 250 bases to about 5000 bases in length. In embodiments, the nucleic acid is about 300 bases to about 5000 bases in length. In embodiments, the nucleic acid is about 350 bases to about 5000 bases in length. In embodiments, the nucleic acid is about 400 bases to about 5000 bases in length. In embodiments, the nucleic acid is about 450 bases to about 5000 bases in length. In embodiments, the nucleic acid is about 500 bases to about 5000 bases in length. In embodiments, the nucleic acid is about 550 bases to about 5000 bases in length. In embodiments, the nucleic acid is about 600 bases to about 5000 bases in length. In embodiments, the nucleic acid is about 650 bases to about 5000 bases in length. In embodiments, the nucleic acid is about 700 bases to about 5000 bases in length. In embodiments, the nucleic acid is about 750 bases to about 5000 bases in length. In embodiments, the nucleic acid is about 800 bases to about 5000 bases in length. In embodiments, the nucleic acid is about 850 bases to about 5000 bases in length. In embodiments, the nucleic acid is about 900 bases to about 5000 bases in length. In embodiments, the nucleic acid is about 950 bases to about 5000 bases in length. In embodiments, the nucleic acid is about 1000 bases to about 5000 bases in length. In embodiments, the nucleic acid is about 1050 bases to about 5000 bases in length. In embodiments, the nucleic acid is about 1100 bases to about 5000 bases in length. In embodiments, the nucleic acid is about 1150 bases to about 5000 bases in length. In embodiments, the nucleic acid is about 1200 bases to about 5000 bases in length. In embodiments, the nucleic acid is about 1250 bases to about 5000 bases in length. In embodiments, the nucleic acid is about 1300 bases to about 5000 bases in length. In embodiments, the nucleic acid is about 1350 bases to about 5000 bases in length. In embodiments, the nucleic acid is about 1400 bases to about 5000 bases in length. In embodiments, the nucleic acid is about 1450 bases to about 5000 bases in length. In embodiments, the nucleic acid is about 1500 bases to about 5000 bases in length. In embodiments, the nucleic acid is about 1550 bases to about 5000 bases in length. In embodiments, the nucleic acid is about 1600 bases to about 5000 bases in length. In embodiments, the nucleic acid is about 1650 bases to about 5000 bases in length. In embodiments, the nucleic acid is about 1700 bases to about 5000 bases in length. In embodiments, the nucleic acid is about 1750 bases to about 5000 bases in length. In embodiments, the nucleic acid is about 1800 bases to about 5000 bases in length. In embodiments, the nucleic acid is about 1850 bases to about 5000 bases in length. In embodiments, the nucleic acid is about 1900 bases to about 5000 bases in length. In embodiments, the nucleic acid is about 1950 bases to about 5000 bases in length. In embodiments, the nucleic acid is about 2000 bases to about 5000 bases in length. In embodiments, the nucleic acid is about 2050 bases to about 5000 bases in length. In embodiments, the nucleic acid is about 2100 bases to about 5000 bases in length. In embodiments, the nucleic acid is about 2150 bases to about 5000 bases in length. In embodiments, the nucleic acid is about 2200 bases to about 5000 bases in length. In embodiments, the nucleic acid is about 2250 bases to about 5000 bases in length. In embodiments, the nucleic acid is about 2300 bases to about 5000 bases in length. In embodiments, the nucleic acid is about 2350 bases to about 5000 bases in length. In embodiments, the nucleic acid is about 2400 bases to about 5000 bases in length. In embodiments, the nucleic acid is about 2450 bases to about 5000 bases in length. In embodiments, the nucleic acid is about 2500 bases to about 5000 bases in length. In embodiments, the nucleic acid is about 2550 bases to about 5000 bases in length. In embodiments, the nucleic acid is about 2600 bases to about 5000 bases in length. In embodiments, the nucleic acid is about 2650 bases to about 5000 bases in length. In embodiments, the nucleic acid is about 2700 bases to about 5000 bases in length. In embodiments, the nucleic acid is about 2750 bases to about 5000 bases in length. In embodiments, the nucleic acid is about 2800 bases to about 5000 bases in length. In embodiments, the nucleic acid is about 2850 bases to about 5000 bases in length. In embodiments, the nucleic acid is about 2900 bases to about 5000 bases in length. In embodiments, the nucleic acid is about 2950 bases to about 5000 bases in length. In embodiments, the nucleic acid is about 3000 bases to about 5000 bases in length. In embodiments, the nucleic acid is about 3050 bases to about 5000 bases in length. In embodiments, the nucleic acid is about 3100 bases to about 5000 bases in length. In embodiments, the nucleic acid is about 3150 bases to about 5000 bases in length. In embodiments, the nucleic acid is about 3200 bases to about 5000 bases in length. In embodiments, the nucleic acid is about 3250 bases to about 5000 bases in length. In embodiments, the nucleic acid is about 3300 bases to about 5000 bases in length. In embodiments, the nucleic acid is about 3350 bases to about 5000 bases in length. In embodiments, the nucleic acid is about 3400 bases to about 5000 bases in length. In embodiments, the nucleic acid is about 3450 bases to about 5000 bases in length. In embodiments, the nucleic acid is about 3500 bases to about 5000 bases in length. In embodiments, the nucleic acid is about 3550 bases to about 5000 bases in length. In embodiments, the nucleic acid is about 3600 bases to about 5000 bases in length. In embodiments, the nucleic acid is about 3650 bases to about 5000 bases in length. In embodiments, the nucleic acid is about 3700 bases to about 5000 bases in length. In embodiments, the nucleic acid is about 3750 bases to about 5000 bases in length. In embodiments, the nucleic acid is about 3800 bases to about 5000 bases in length. In embodiments, the nucleic acid is about 3850 bases to about 5000 bases in length. In embodiments, the nucleic acid is about 3900 bases to about 5000 bases in length. In embodiments, the nucleic acid is about 3950 bases to about 5000 bases in length. In embodiments, the nucleic acid is about 4000 bases to about 5000 bases in length. In embodiments, the nucleic acid is about 4050 bases to about 5000 bases in length. In embodiments, the nucleic acid is about 4100 bases to about 5000 bases in length. In embodiments, the nucleic acid is about 4150 bases to about 5000 bases in length. In embodiments, the nucleic acid is about 4200 bases to about 5000 bases in length. In embodiments, the nucleic acid is about 4250 bases to about 5000 bases in length. In embodiments, the nucleic acid is about 4300 bases to about 5000 bases in length. In embodiments, the nucleic acid is about 4350 bases to about 5000 bases in length. In embodiments, the nucleic acid is about 4400 bases to about 5000 bases in length. In embodiments, the nucleic acid is about 4450 bases to about 5000 bases in length. In embodiments, the nucleic acid is about 4500 bases to about 5000 bases in length. In embodiments, the nucleic acid is about 4550 bases to about 5000 bases in length. In embodiments, the nucleic acid is about 4600 bases to about 5000 bases in length. In embodiments, the nucleic acid is about 4650 bases to about 5000 bases in length. In embodiments, the nucleic acid is about 4700 bases to about 5000 bases in length. In embodiments, the nucleic acid is about 4750 bases to about 5000 bases in length. In embodiments, the nucleic acid is about 4800 bases to about 5000 bases in length. In embodiments, the nucleic acid is about 4850 bases to about 5000 bases in length. In embodiments, the nucleic acid is about 4900 bases to about 5000 bases in length. In embodiments, the nucleic acid is about 4950 bases to about 5000 bases in length.

In embodiments, the nucleic acid is about 20 bases to about 4950 bases in length. In embodiments, the nucleic acid is about 20 bases to about 4900 bases in length. In embodiments, the nucleic acid is about 20 bases to about 4850 bases in length. In embodiments, the nucleic acid is about 20 bases to about 4800 bases in length. In embodiments, the nucleic acid is about 20 bases to about 4750 bases in length. In embodiments, the nucleic acid is about 20 bases to about 4700 bases in length. In embodiments, the nucleic acid is about 20 bases to about 4650 bases in length. In embodiments, the nucleic acid is about 20 bases to about 4600 bases in length. In embodiments, the nucleic acid is about 20 bases to about 4550 bases in length. In embodiments, the nucleic acid is about 20 bases to about 4500 bases in length. In embodiments, the nucleic acid is about 20 bases to about 4450 bases in length. In embodiments, the nucleic acid is about 20 bases to about 4400 bases in length. In embodiments, the nucleic acid is about 20 bases to about 4350 bases in length. In embodiments, the nucleic acid is about 20 bases to about 4300 bases in length. In embodiments, the nucleic acid is about 20 bases to about 4250 bases in length. In embodiments, the nucleic acid is about 20 bases to about 4200 bases in length. In embodiments, the nucleic acid is about 20 bases to about 4150 bases in length. In embodiments, the nucleic acid is about 20 bases to about 4100 bases in length. In embodiments, the nucleic acid is about 20 bases to about 4050 bases in length. In embodiments, the nucleic acid is about 20 bases to about 4000 bases in length. In embodiments, the nucleic acid is about 20 bases to about 3950 bases in length. In embodiments, the nucleic acid is about 20 bases to about 3900 bases in length. In embodiments, the nucleic acid is about 20 bases to about 3850 bases in length. In embodiments, the nucleic acid is about 20 bases to about 3800 bases in length. In embodiments, the nucleic acid is about 20 bases to about 3750 bases in length. In embodiments, the nucleic acid is about 20 bases to about 3700 bases in length. In embodiments, the nucleic acid is about 20 bases to about 3650 bases in length. In embodiments, the nucleic acid is about 20 bases to about 3600 bases in length. In embodiments, the nucleic acid is about 20 bases to about 3550 bases in length. In embodiments, the nucleic acid is about 20 bases to about 3500 bases in length. In embodiments, the nucleic acid is about 20 bases to about 3450 bases in length. In embodiments, the nucleic acid is about 20 bases to about 3400 bases in length. In embodiments, the nucleic acid is about 20 bases to about 3350 bases in length. In embodiments, the nucleic acid is about 20 bases to about 3300 bases in length. In embodiments, the nucleic acid is about 20 bases to about 3250 bases in length. In embodiments, the nucleic acid is about 20 bases to about 3200 bases in length. In embodiments, the nucleic acid is about 20 bases to about 3150 bases in length. In embodiments, the nucleic acid is about 20 bases to about 3100 bases in length. In embodiments, the nucleic acid is about 20 bases to about 3050 bases in length. In embodiments, the nucleic acid is about 20 bases to about 3000 bases in length. In embodiments, the nucleic acid is about 20 bases to about 2950 bases in length. In embodiments, the nucleic acid is about 20 bases to about 2900 bases in length. In embodiments, the nucleic acid is about 20 bases to about 2850 bases in length. In embodiments, the nucleic acid is about 20 bases to about 2800 bases in length. In embodiments, the nucleic acid is about 20 bases to about 2750 bases in length. In embodiments, the nucleic acid is about 20 bases to about 2700 bases in length. In embodiments, the nucleic acid is about 20 bases to about 2650 bases in length. In embodiments, the nucleic acid is about 20 bases to about 2600 bases in length. In embodiments, the nucleic acid is about 20 bases to about 2550 bases in length. In embodiments, the nucleic acid is about 20 bases to about 2500 bases in length. In embodiments, the nucleic acid is about 20 bases to about 2450 bases in length. In embodiments, the nucleic acid is about 20 bases to about 2400 bases in length. In embodiments, the nucleic acid is about 20 bases to about 2350 bases in length. In embodiments, the nucleic acid is about 20 bases to about 2300 bases in length. In embodiments, the nucleic acid is about 20 bases to about 2250 bases in length. In embodiments, the nucleic acid is about 20 bases to about 2200 bases in length. In embodiments, the nucleic acid is about 20 bases to about 2150 bases in length. In embodiments, the nucleic acid is about 20 bases to about 2100 bases in length. In embodiments, the nucleic acid is about 20 bases to about 2050 bases in length. In embodiments, the nucleic acid is about 20 bases to about 2000 bases in length. In embodiments, the nucleic acid is about 20 bases to about 1950 bases in length. In embodiments, the nucleic acid is about 20 bases to about 1900 bases in length. In embodiments, the nucleic acid is about 20 bases to about 1850 bases in length. In embodiments, the nucleic acid is about 20 bases to about 1800 bases in length. In embodiments, the nucleic acid is about 20 bases to about 1750 bases in length. In embodiments, the nucleic acid is about 20 bases to about 1700 bases in length. In embodiments, the nucleic acid is about 20 bases to about 1650 bases in length. In embodiments, the nucleic acid is about 20 bases to about 1600 bases in length. In embodiments, the nucleic acid is about 20 bases to about 1550 bases in length. In embodiments, the nucleic acid is about 20 bases to about 1500 bases in length. In embodiments, the nucleic acid is about 20 bases to about 1450 bases in length. In embodiments, the nucleic acid is about 20 bases to about 1400 bases in length. In embodiments, the nucleic acid is about 20 bases to about 1350 bases in length. In embodiments, the nucleic acid is about 20 bases to about 1300 bases in length. In embodiments, the nucleic acid is about 20 bases to about 1250 bases in length. In embodiments, the nucleic acid is about 20 bases to about 1200 bases in length. In embodiments, the nucleic acid is about 20 bases to about 1150 bases in length. In embodiments, the nucleic acid is about 20 bases to about 1100 bases in length. In embodiments, the nucleic acid is about 20 bases to about 1050 bases in length. In embodiments, the nucleic acid is about 20 bases to about 1000 bases in length. In embodiments, the nucleic acid is about 20 bases to about 950 bases in length. In embodiments, the nucleic acid is about 20 bases to about 900 bases in length. In embodiments, the nucleic acid is about 20 bases to about 850 bases in length. In embodiments, the nucleic acid is about 20 bases to about 800 bases in length. In embodiments, the nucleic acid is about 20 bases to about 750 bases in length. In embodiments, the nucleic acid is about 20 bases to about 700 bases in length. In embodiments, the nucleic acid is about 20 bases to about 650 bases in length. In embodiments, the nucleic acid is about 20 bases to about 600 bases in length. In embodiments, the nucleic acid is about 20 bases to about 550 bases in length. In embodiments, the nucleic acid is about 20 bases to about 500 bases in length. In embodiments, the nucleic acid is about 20 bases to about 450 bases in length. In embodiments, the nucleic acid is about 20 bases to about 400 bases in length. In embodiments, the nucleic acid is about 20 bases to about 350 bases in length. In embodiments, the nucleic acid is about 20 bases to about 300 bases in length. In embodiments, the nucleic acid is about 20 bases to about 250 bases in length. In embodiments, the nucleic acid is about 20 bases to about 200 bases in length. In embodiments, the nucleic acid is about 20 bases to about 150 bases in length. In embodiments, the nucleic acid is about 20 bases to about 100 bases in length. In embodiments, the nucleic acid is about 20 bases to about 50 bases in length. In embodiments, the nucleic acid is about 20, 50, 100, 200, 250, 300, 350, 400, 450, 500, 550, 600, 650, 700, 750, 800, 850, 900, 950, 1000, 1050, 1100, 1200, 1250, 1300, 1350, 1400, 1450, 1500, 1550, 1600, 1650, 1700, 1750, 1800, 1850, 1900, 1950, 2000, 2050, 2100, 2200, 2250, 2300, 2350, 2400, 2450, 2500, 2550, 2600, 2650, 7200, 2750, 2800, 2850, 2900, 2950, 3000, 3050, 3100, 3200, 3250, 3300, 3350, 3400, 3450, 3500, 3550, 3600, 3650, 3700, 3750, 3800, 3850, 3900, 3950, 4000, 4050, 4100, 4200, 4250, 4300, 4350, 4400, 4450, 4500, 4550, 4600, 4650, 4700, 4750, 4800, 4850, 4900, 4950, or 5000 bases in length.

In embodiments, the nucleic acid is from about 20 bases to about 1000 bases in length. In embodiments, the nucleic acid is from about 100 bases to about 1000 bases in length. In embodiments, the nucleic acid is from about 200 bases to about 1000 bases in length. In embodiments, the nucleic acid is from about 300 bases to about 1000 bases in length. In embodiments, the nucleic acid is from about 400 bases to about 1000 bases in length. In embodiments, the nucleic acid is from about 500 bases to about 1000 bases in length. In embodiments, the nucleic acid is from about 600 bases to about 1000 bases in length. In embodiments, the nucleic acid is from about 700 bases to about 1000 bases in length. In embodiments, the nucleic acid is from about 800 bases to about 1000 bases in length. In embodiments, the nucleic acid is from about 900 bases to about 1000 bases in length.

In embodiments, the nucleic acid is from about 20 bases to about 900 bases in length. In embodiments, the nucleic acid is from about 20 bases to about 800 bases in length. In embodiments, the nucleic acid is from about 20 bases to about 700 bases in length. In embodiments, the nucleic acid is from about 20 bases to about 600 bases in length. In embodiments, the nucleic acid is from about 20 bases to about 500 bases in length. In embodiments, the nucleic acid is from about 20 bases to about 400 bases in length. In embodiments, the nucleic acid is from about 20 bases to about 300 bases in length. In embodiments, the nucleic acid is from about 20 bases to about 200 bases in length. In embodiments, the nucleic acid is from about 20 bases to about 100 bases in length. In embodiments, the nucleic acid is about 20, 100, 200, 300, 400, 500, 600, 700, 800, 900, or 1000 bases in length.

For the methods provided herein, in embodiments, the nucleic acid includes a donor DNA that in instances is inserted into the genome of the T cell, thereby producing the engineered T cell. Thus, in embodiments, the nucleic acid includes a donor DNA. In embodiments, the nucleic acid is a double-stranded circular DNA (e.g. a plasmid). The donor DNA may be delivered into the cell using a variety of methods including transfection, electroporation, and viral transduction methods. Thus, in embodiments, the nucleic acid is a plasmid, a nanoplasmid, a minicircle, a linear plasmid, or a viral vector including the donor DNA. In embodiments, the nucleic acid is a plasmid including the donor DNA. In embodiments, the nucleic acid is a nanoplasmid including the donor DNA. In embodiments, the nucleic acid is a minicircle including the donor DNA. In embodiments, the nucleic acid is a viral vector including the donor DNA. In embodiments, the DNA is not a linear DNA (e.g. a double-stranded linear DNA). In embodiments, the DNA is a circular DNA (e.g. a double-stranded circular DNA). In embodiments, the DNA is a linear DNA. In embodiments, the DNA is single stranded DNA.

The methods provided herein are useful for making T cell receptor (TCR) engineered T cells. For example, the methods may be used to replace (e.g. knock-out) an endogenous TCR with a tumor specific antigen associated TCR (e.g. knock-in) in a T cell. Thus, in embodiments, the donor DNA encodes an exogenous T cell receptor (TCR)-alpha or a fragment thereof, an exogenous TCR-beta or a fragment thereof, or a combination thereof. In embodiments, the donor DNA encodes an exogenous T cell receptor (TCR)-alpha or a fragment thereof. In embodiments, the donor DNA encodes an exogenous TCR-beta or a fragment thereof.

In embodiments, the donor DNA is about 5 to about 1000 bases in length. In embodiments, the donor DNA is about 100 to about 1000 bases in length. In embodiments, the donor DNA is about 200 to about 1000 bases in length. In embodiments, the donor DNA is about 300 to about 1000 bases in length. In embodiments, the donor DNA is about 400 to about 1000 bases in length. In embodiments, the donor DNA is about 500 to about 1000 bases in length. In embodiments, the donor DNA is about 600 to about 1000 bases in length. In embodiments, the donor DNA is about 700 to about 1000 bases in length. In embodiments, the donor DNA is about 800 to about 1000 bases in length. In embodiments, the donor DNA is about 900 to about 1000 bases in length.

In embodiments, the donor DNA is about 5 to about 900 bases in length. In embodiments, the donor DNA is about 5 to about 800 bases in length. In embodiments, the donor DNA is about 5 to about 700 bases in length. In embodiments, the donor DNA is about 5 to about 600 bases in length. In embodiments, the donor DNA is about 5 to about 500 bases in length. In embodiments, the donor DNA is about 5 to about 400 bases in length. In embodiments, the donor DNA is about 5 to about 300 bases in length. In embodiments, the donor DNA is about 5 to about 200 bases in length. In embodiments, the donor DNA is about 5 to about 100 bases in length. In embodiments, the donor DNA is about 5, 100, 200, 300, 400, 500, 600, 700, 800, 900 or 1000 bases in length.

In embodiments, the donor DNA is about 50 to about 600 bases in length. In embodiments, the donor DNA is about 100 to about 600 bases in length. In embodiments, the donor DNA is about 150 to about 600 bases in length. In embodiments, the donor DNA is about 200 to about 600 bases in length. In embodiments, the donor DNA is about 250 to about 600 bases in length. In embodiments, the donor DNA is about 300 to about 600 bases in length. In embodiments, the donor DNA is about 350 to about 600 bases in length. In embodiments, the donor DNA is about 400 to about 600 bases in length. In embodiments, the donor DNA is about 450 to about 600 bases in length. In embodiments, the donor DNA is about 500 to about 600 bases in length. In embodiments, the donor DNA is about 550 to about 600 bases in length.

In embodiments, the donor DNA is about 50 to about 550 bases in length. In embodiments, the donor DNA is about 50 to about 500 bases in length. In embodiments, the donor DNA is about 50 to about 450 bases in length. In embodiments, the donor DNA is about 50 to about 400 bases in length. In embodiments, the donor DNA is about 50 to about 350 bases in length. In embodiments, the donor DNA is about 50 to about 300 bases in length. In embodiments, the donor DNA is about 50 to about 250 bases in length. In embodiments, the donor DNA is about 50 to about 200 bases in length. In embodiments, the donor DNA is about 50 to about 150 bases in length. In embodiments, the donor DNA is about 150 to about 100 bases in length. In embodiments, the donor DNA is about 50, 100, 150, 200, 250, 300, 350, 400, 450, 500, 550, or 600 bases in length.

In embodiments, the donor DNA is about 20 bases to about 5000 bases in length. In embodiments, the donor DNA is about 250 bases to about 5000 bases in length. In embodiments, the donor DNA is about 500 bases to about 5000 bases in length. In embodiments, the donor DNA is about 750 bases to about 5000 bases in length. In embodiments, the donor DNA is about 1000 bases to about 5000 bases in length. In embodiments, the donor DNA is about 1250 bases to about 5000 bases in length. In embodiments, the donor DNA is about 1500 bases to about 5000 bases in length. In embodiments, the donor DNA is about 1750 bases to about 5000 bases in length. In embodiments, the donor DNA is about 2000 bases to about 5000 bases in length. In embodiments, the donor DNA is about 2250 bases to about 5000 bases in length. In embodiments, the donor DNA is about 2500 bases to about 5000 bases in length. In embodiments, the donor DNA is about 2750 bases to about 5000 bases in length. In embodiments, the donor DNA is about 3000 bases to about 5000 bases in length. In embodiments, the donor DNA is about 3250 bases to about 5000 bases in length. In embodiments, the donor DNA is about 3500 bases to about 5000 bases in length. In embodiments, the donor DNA is about 3750 bases to about 5000 bases in length. In embodiments, the donor DNA is about 4000 bases to about 5000 bases in length. In embodiments, the donor DNA is about 4250 bases to about 5000 bases in length. In embodiments, the donor DNA is about 4500 bases to about 5000 bases in length. In embodiments, the donor DNA is about 4750 bases to about 5000 bases in length.

In embodiments, the donor DNA is about 20 bases to about 4750 bases in length. In embodiments, the donor DNA is about 20 bases to about 4500 bases in length. In embodiments, the donor DNA is about 20 bases to about 4250 bases in length. In embodiments, the donor DNA is about 20 bases to about 4000 bases in length. In embodiments, the donor DNA is about 20 bases to about 3750 bases in length. In embodiments, the donor DNA is about 20 bases to about 3500 bases in length. In embodiments, the donor DNA is about 20 bases to about 3250 bases in length. In embodiments, the donor DNA is about 20 bases to about 3000 bases in length. In embodiments, the donor DNA is about 20 bases to about 2750 bases in length. In embodiments, the donor DNA is about 20 bases to about 2500 bases in length. In embodiments, the donor DNA is about 20 bases to about 2250 bases in length. In embodiments, the donor DNA is about 20 bases to about 2000 bases in length. In embodiments, the donor DNA is about 20 bases to about 1750 bases in length. In embodiments, the donor DNA is about 20 bases to about 1500 bases in length. In embodiments, the donor DNA is about 20 bases to about 1250 bases in length. In embodiments, the donor DNA is about 20 bases to about 1000 bases in length. In embodiments, the donor DNA is about 20 bases to about 750 bases in length. In embodiments, the donor DNA is about 20 bases to about 500 bases in length. In embodiments, the donor DNA is about 20 bases to about 250 bases in length. In embodiments, the donor DNA is about 20, 250, 500, 750, 1000, 1250, 1500, 1750, 2000, 2250, 2500, 2750, 3000, 3250, 3500, 3750, 4000, 4250, 4500, 4750, or 5000 bases in length.

In instances, delivery of the nucleic acid into the T cell may be facilitated by a delivery vehicle. The delivery vehicle may facilitate interaction of the nucleic acid and the T cell membrane, thereby allowing entry of the nucleic acid into the T cell. In one example, the nucleic acid may be encapsulated in the delivery vehicle. In another example, the nucleic acid may be non-covalently associated with the delivery vehicle. Thus, in embodiments, the nucleic acid is associated with a delivery vehicle. In embodiments, the delivery vehicle is a lipid particle or a nanoparticle. In embodiments, the delivery vehicle is a lipid particle. In embodiments, the delivery vehicle is a nanoparticle. In embodiments, the delivery vehicle is a liposome or a lipid nanoparticle.

For the methods provided herein, in embodiments, the T cell is a primary T cell. “Primary T cell” is used in accordance to its ordinary meaning in the biological arts and refers to a T cell that is directly expanded from a T cell extracted from a subject.

The methods provided herein including embodiments thereof may include contacting the T cell with a gene editing reagent, thereby allowing editing of a target gene within the T cell. For example, the gene editing reagent may facilitate knock-out of an endogenous gene (e.g. endogenous TCR) and knock-in of an tumor antigen specific TCR. Thus, in embodiments, the method further includes contacting the T cell with a gene editing reagent. In embodiments, contacting the T cell with the gene editing reagent includes contacting the T cell with a nucleic acid sequence encoding the gene editing reagent. In embodiments, the T cell is contacted with the nucleic acid in the presence of the gene editing agent or the nucleic acid sequence encoding the gene editing reagent. In embodiments, the T cell is contacted with the nucleic acid in the presence of the gene editing reagent. In embodiments, the T cell is contacted with the nucleic acid in the presence of the nucleic acid sequence encoding the gene editing reagent.

In embodiments, the gene editing reagent includes an RNA-guided nuclease. In embodiments, the RNA-guided nuclease is a CRISPR-Cas system. In embodiments, the CRISPR-Cas system includes Cas1, Cas1B, Cas2, Cas3, Cas4, Cas5, Cas6, Cas7, Cas8, Cas9, Cas10, Cas12, Cas13, nCas9, Cas-CLOVER, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4. In embodiments, the CRISPR-Cas system includes Cas1. In embodiments, the CRISPR-Cas system includes Cas1B. In embodiments, the CRISPR-Cas system includes Cas2. In embodiments, the CRISPR-Cas system includes Cas3. In embodiments, the CRISPR-Cas system includes Cas4. In embodiments, the CRISPR-Cas system includes Cas5. In embodiments, the CRISPR-Cas system includes Cas6. In embodiments, the CRISPR-Cas system includes Cas7. In embodiments, the CRISPR-Cas system includes Cas8. In embodiments, the CRISPR-Cas system includes Cas9. In embodiments, the CRISPR-Cas system includes Cas10. In embodiments, the CRISPR-Cas system includes Cas12. In embodiments, the CRISPR-Cas system includes Cas13. In embodiments, the CRISPR-Cas system includes nCas9. In embodiments, the CRISPR-Cas system includes Cas-CLOVER. In embodiments, the CRISPR-Cas system includes Csy1. In embodiments, the CRISPR-Cas system includes Csy2. In embodiments, the CRISPR-Cas system includes Csy3. In embodiments, the CRISPR-Cas system includes Cse1. In embodiments, the CRISPR-Cas system includes Cse2. In embodiments, the CRISPR-Cas system includes Csc1. In embodiments, the CRISPR-Cas system includes Csc2. In embodiments, the CRISPR-Cas system includes Csm2. In embodiments, the CRISPR-Cas system includes Csm3. In embodiments, the CRISPR-Cas system includes Csm4. In embodiments, the CRISPR-Cas system includes Csm5. In embodiments, the CRISPR-Cas system includes Csm6. In embodiments, the CRISPR-Cas system includes Cmr1. In embodiments, the CRISPR-Cas system includes Cmr3. In embodiments, the CRISPR-Cas system includes Cmr4. In embodiments, the CRISPR-Cas system includes Cmr5. In embodiments, the CRISPR-Cas system includes Cmr6. In embodiments, the CRISPR-Cas system includes Csb1. In embodiments, the CRISPR-Cas system includes Csb3. In embodiments, the CRISPR-Cas system includes Csx17. In embodiments, the CRISPR-Cas system includes Csx14. In embodiments, the CRISPR-Cas system includes Csx10. In embodiments, the CRISPR-Cas system includes Csx16. In embodiments, the CRISPR-Cas system includes CsaX. In embodiments, the CRISPR-Cas system includes Csx3. In embodiments, the CRISPR-Cas system includes Csx1. In embodiments, the CRISPR-Cas system includes Csx15. In embodiments, the CRISPR-Cas system includes Csf1. In embodiments, the CRISPR-Cas system includes Csf2. In embodiments, the CRISPR-Cas system includes Csf3. In embodiments, the CRISPR-Cas system includes Csf4. In embodiments, the gene editing reagent further includes a guide RNA (gRNA).

In embodiments, the gene editing reagent is MAD7, a TALEN, or a ZFN. In embodiments, the gene editing reagent is MAD7. In embodiments, the gene editing reagent is a TALEN. In embodiments, the gene editing reagent is a ZFN. MAD7 is an engineered nuclease of the Class 2 type V-A CRISPR-Cas (Cas12a/Cpf1) family (refseq WP_055225123.1). See, e.g., CRISPR J. April 2020; 3 (2): 97-108, which is incorporated herein by reference in its entirety.

As described throughout the specification, including in the figures and examples, the donor DNA may be inserted into a TCR locus of a T cell, thereby forming an engineered T cell. Thus, for the methods provided herein, in embodiments, the donor DNA is inserted into an endogenous TCR locus. In embodiments, the endogenous TCR locus is an endogenous TCR-alpha locus, an endogenous TCR-beta locus, or a combination thereof. In embodiments, the endogenous TCR locus is an endogenous TCR-alpha locus. In embodiments, the endogenous TCR locus is an endogenous TCR-beta locus.

For the methods provided herein, the gene editing reagent and the nucleic acid may be delivered into the T cell using a variety of methods known in the art, including but not limited to electroporation and transfection methods. In embodiments, contacting the T cell with the gene editing reagent includes transfecting the T cell with the gene editing reagent. In embodiments, contacting the T cell with the nucleic acid includes transfecting the T cell with the nucleic acid.

As described above, the one or more cGAS-STING pathway inhibitors may be any compound that inhibits or downregulates the activity or production of any component of the cGAS-STING axis, including a component of the cGAS pathway, the STING pathway, or TBK1. In embodiments, the one or more cGAS-STING pathway inhibitors includes a cGAS inhibitor. In embodiments, the one or more cGAS-STING pathway inhibitors includes a STING inhibitor. In embodiments, the one or more cGAS-STING pathway inhibitors includes a TANK-binding kinase 1 (TBK1) inhibitor. The cGAS, STING, or TBK1 inhibitor may include a kinase inhibitor. As used herein, “TANK-binding kinase 1 inhibitor” or “TBK1 inhibitor” refers to a compound that decreases or downregulates the activity or production of TANK-binding kinase 1. For example, the TBK1 inhibitor may inhibit phosphorylation of TBK1 or a TBK1 target. In embodiments, the TBK1 inhibitor is Amlexanox, BX795, or MRT67307.

In embodiments, the one or more cGAS-STING pathway inhibitors is Amlexanox (Aml), MRT67307 (MRT), BX795, H151, ODN-A151 (ODN151), Ru.521, G140, or combinations thereof. In embodiments, the one or more cGAS-STING pathway inhibitors includes Aml. In embodiments, the one or more cGAS-STING pathway inhibitors includes MRT. In embodiments, the one or more cGAS-STING pathway inhibitors includes BX795. In embodiments, the one or more cGAS-STING pathway inhibitors includes H151. In embodiments, the one or more cGAS-STING pathway inhibitors includes ODN151. In embodiments, the one or more cGAS-STING pathway inhibitors includes Ru.521. In embodiments, the one or more cGAS-STING pathway inhibitors includes G140. In embodiments, the one or more cGAS-STING pathway inhibitors includes a compound set forth in Table 5.

As used herein, ODN-A151 (ODN151) refers to a nucleic sequence including 5′-TTAGGGTTAGGGTTAGGGTTAGGG-3 (SEQ ID NO:1). In embodiments, ODN-A151 is the nucleic acid sequence of SEQ ID NO: 1. In embodiments, one or more internucleotide linkages of SEQ ID NO: 1 are through a phosphorothioate moiety (thiophosphate) moiety. The phosphorothioate moiety may be a monothiophosphate (—P(O)₃(S)₃⁻—) or a dithiophosphate (—P(O)₂(S)₂³⁻—). In embodiments, the phosphorothioate moiety is a monothiophosphate (—P(O)₃(S)₃⁻—).

In embodiments, the one or more cGAS-STING pathway inhibitors is Aml. In embodiments, the one or more cGAS-STING pathway inhibitors is MRT. In embodiments, the one or more cGAS-STING pathway inhibitors is BX795. In embodiments, the one or more cGAS-STING pathway inhibitors is H151. In embodiments, the one or more cGAS-STING pathway inhibitors is ODN151. In embodiments, the one or more cGAS-STING pathway inhibitors is Ru.521. In embodiments, the one or more cGAS-STING pathway inhibitors is G140.

In embodiments, the cGAS-STING pathway inhibitor is Aml. In embodiments, the cGAS-STING pathway inhibitor is MRT. In embodiments, the cGAS-STING pathway inhibitor is BX795. In embodiments, the cGAS-STING pathway inhibitor is H151. In embodiments, the cGAS-STING pathway inhibitor is ODN151. In embodiments, the cGAS-STING pathway inhibitor is Ru.521. In embodiments, the cGAS-STING pathway inhibitor is G140. In embodiments, the cGAS-STING pathway inhibitor is Aml and no other cGAS-STING inhibitor. In embodiments, the cGAS-STING pathway inhibitor is MRT and no other cGAS-STING inhibitor. In embodiments, the cGAS-STING pathway inhibitor is BX795 and no other cGAS-STING inhibitor. In embodiments, the cGAS-STING pathway inhibitor is H151 and no other cGAS-STING inhibitor. In embodiments, the cGAS-STING pathway inhibitor is ODN151 and no other cGAS-STING inhibitor. In embodiments, the cGAS-STING pathway inhibitor is Ru.521 and no other cGAS-STING inhibitor. In embodiments, the cGAS-STING pathway inhibitor is G140 and no other cGAS-STING inhibitor.

In embodiments, the one or more cGAS-STING pathway inhibitors is selected from: Aml, BX795, ODN151, and MRT. In embodiments, the one or more cGAS-STING pathway inhibitors is ODN151. In embodiments, the one or more cGAS-STING pathway inhibitors is BX795. In embodiments, the one or more cGAS-STING pathway inhibitors includes at least one cGAS-STING pathway inhibitor. In embodiments, the one or more cGAS-STING pathway inhibitors includes a combination of cGAS-STING pathway inhibitors. In embodiments, the cGAS-STING pathway inhibitor does not include more than one cGAS-STING pathway inhibitor.

Applicant has discovered that treatment of the T cell with one or more cGAS-STING pathway inhibitors prior to contacting the T cell with the nucleic acid or in the presence of the nucleic acid is effective for generating an engineered T cell. In embodiments, the T cell and the nucleic acid are contacted in the presence of one or more cGAS-STING pathway inhibitors. For example, the T cell may be transfected with the nucleic acid in the presence of the one or more cGAS-STING pathway inhibitors. In another example, the T cell may be electroporated with the nucleic acid in the presence of the one or more cGAS-STING pathway inhibitors. In embodiments, the T cell is contacted sequentially with the nucleic acid and the one or more cGAS-STING pathway inhibitors. In embodiments, T cell is contacted with the one or more cGAS-STING pathway inhibitors prior to the nucleic acid. For example, the one or more cGAS-STING pathway inhibitors may be added to the T cell culture prior to transfecting the T cells with the nucleic acid.

In embodiments, the T cell is contacted with the one or more cGAS-STING pathway inhibitors for up to about 10 hours. In embodiments, the T cell is contacted with the one or more cGAS-STING pathway inhibitors for about 1 hours to about 10 hours. In embodiments, the T cell is contacted with the one or more cGAS-STING pathway inhibitors for about 2 hours to about 10 hours. In embodiments, the T cell is contacted with the one or more cGAS-STING pathway inhibitors for about 3 hours to about 10 hours. In embodiments, the T cell is contacted with the one or more cGAS-STING pathway inhibitors for about 4 hours to about 10 hours. In embodiments, the T cell is contacted with the one or more cGAS-STING pathway inhibitors for about 5 hours to about 10 hours. In embodiments, the T cell is contacted with the one or more cGAS-STING pathway inhibitors for about 6 hours to about 10 hours. In embodiments, the T cell is contacted with the one or more cGAS-STING pathway inhibitors for about 7 hours to about 10 hours. In embodiments, the T cell is contacted with the one or more cGAS-STING pathway inhibitors for about 8 hours to about 10 hours. In embodiments, the T cell is contacted with the one or more cGAS-STING pathway inhibitors for about 9 hours to about 10 hours.

In embodiments, the T cell is contacted with the one or more cGAS-STING pathway inhibitors for up to about 9 hours. In embodiments, the T cell is contacted with the one or more cGAS-STING pathway inhibitors for about 1 hours to about 9 hours. In embodiments, the T cell is contacted with the one or more cGAS-STING pathway inhibitors for about 2 hours to about 9 hours. In embodiments, the T cell is contacted with the one or more cGAS-STING pathway inhibitors for about 2 hours to about 8 hours. In embodiments, the T cell is contacted with the one or more cGAS-STING pathway inhibitors for about 2 hours to about 7 hours. In embodiments, the T cell is contacted with the one or more cGAS-STING pathway inhibitors for about 2 hours to about 6 hours. In embodiments, the T cell is contacted with the one or more cGAS-STING pathway inhibitors for about 2 hours to about 5 hours. In embodiments, the T cell is contacted with the one or more cGAS-STING pathway inhibitors for about 2 hours to about 4 hours. In embodiments, the T cell is contacted with the one or more cGAS-STING pathway inhibitors for about 2 hours to about 3 hours. In embodiments, the T cell is contacted with the one or more cGAS-STING pathway inhibitors for about 2 hours, 3 hours, 4 hours, 5 hours, 6 hours, 7 hours, 8 hours, 9 hours, or 10 hours. In embodiments, the T cell is contacted with the one or more cGAS-STING pathway inhibitors for about 6 hours. In embodiments, the T cell is contacted with the one or more cGAS-STING pathway inhibitors for 6 hours.

For the methods provided herein, in embodiments, the T cell is contacted with one cGAS-STING pathway inhibitor. In embodiments, the T cell is contacted with one cGAS-STING pathway inhibitor and no other cGAS-STING pathway inhibitors. In embodiments, the cGAS-STING pathway inhibitor is a cGAS-STING pathway inhibitor provided herein and does not include any other cGAS-STING pathway inhibitor.

Provided herein, inter alia, are methods for increasing the viability of engineered T cells, including contacting T cells with a nucleic acid and one or more cGAS-STING pathway inhibitors thereby forming the engineered T cells, wherein the engineered T cells have increased cell viability relative to engineered T cells that are formed without contacting T cells with the one or more cGAS-STING pathway inhibitors. Methods provided herein are useful for overcoming toxicity associated with engineering T cells, including toxicity associated with delivering a nucleic acid into a T cell or the presence of a nucleic acid in the cytoplasm of the T cell. Thus, the methods are contemplated to improve viability of engineered T cells. “Cell viability” is used in accordance to its ordinary meaning in the arts and refers to the number or proportion of living cells within a population of cells. Cell viability may be assessed by measuring cell proliferation, cell membrane integrity, cell function, or metabolic activity. Cell viability may be measured by contacting the cells with a nucleic acid binding dye that only enter cells with compromised or damaged cell membrane. Cell viability may be measured by contacting the cells with reagents that react with enzymes in live cells, or reagents that detect cellular redox potential. For example, cell viability may be measured using fluorescence detection assays, including assays using one or more of acridine orange, 4′,6-diamidino-2-phenylindole (DAPI), propidium iodide (PI), or SYTOX Blue nucleic acid stain, etc. In embodiments, cell viability may be measured by fluorescence microscopy or flow cytometry. Thus, in an aspect is provided a method of increasing cell viability of a population of engineered T cells, including contacting a population of T cells with one or more cyclic GMP-AMP synthase (cGAS)-stimulator of interferon gene (STING) pathway inhibitors and a nucleic acid, thereby forming the population of engineered T cells, wherein the population of engineered T cells has increased cell viability relative to a population of engineered T cells that are not contacted with one or more cGAS-STING pathway inhibitors.

In embodiments, the nucleic acid includes a donor DNA. In embodiments, the nucleic acid is a double-stranded circular DNA (e.g. a plasmid). In embodiments, the nucleic acid is a plasmid, a nanoplasmid, a minicircle, or a viral vector including a donor DNA. In embodiments, the donor DNA encodes an exogenous T cell receptor (TCR)-beta or a fragment thereof, an exogenous TCR-alpha or a fragment thereof, or a combination thereof. In embodiments, the nucleic acid is associated with a delivery vehicle. In embodiments, the delivery vehicle is a lipid particle or a nanoparticle.

For the methods provided herein, in embodiments, the T cell is a primary T cell.

For the methods provided herein, in embodiments, the method further includes contacting the population of T cells with a gene editing reagent. In embodiments, contacting the population of T cells with the gene editing reagent includes contacting the population of T cells with a nucleic acid sequence encoding the gene editing reagent. In embodiments, the T cell is contacted with the nucleic acid in the presence of the gene editing agent or the nucleic acid sequence encoding the gene editing reagent.

In embodiments, the donor DNA is inserted into an endogenous TCR locus. In embodiments, the endogenous TCR locus is an endogenous TCR-alpha locus, an endogenous TCR-beta locus, or a combination thereof.

In embodiments, contacting the T cell with the gene editing reagent includes transfecting the T cell with the gene editing reagent.

In embodiments, the one or more cGAS-STING pathway inhibitors includes a cGAS inhibitor. In embodiments, the one or more cGAS-STING pathway inhibitors includes a STING inhibitor. In embodiments, the one or more cGAS-STING pathway inhibitors includes a TANK-binding kinase 1 (TBK1) inhibitor. In embodiments, the one or more cGAS-STING pathway inhibitors is Amlexanox (Aml), MRT67307 (MRT), BX795, H151, ODN-A151 (ODN151), Ru.521, G140, or combinations thereof. In embodiments, the one or more cGAS-STING pathway inhibitors includes Aml. In embodiments, the one or more cGAS-STING pathway inhibitors includes MRT. In embodiments, the one or more cGAS-STING pathway inhibitors includes BX795. In embodiments, the one or more cGAS-STING pathway inhibitors includes H151. In embodiments, the one or more cGAS-STING pathway inhibitors includes ODN-A151. In embodiments, the one or more cGAS-STING pathway inhibitors includes Ru.521. In embodiments, the one or more cGAS-STING pathway inhibitors includes G140. In embodiments, the one or more cGAS-STING pathway inhibitors is Aml. In embodiments, the one or more cGAS-STING pathway inhibitors is MRT. In embodiments, the one or more cGAS-STING pathway inhibitors is BX795. In embodiments, the one or more cGAS-STING pathway inhibitors is H151. In embodiments, the one or more cGAS-STING pathway inhibitors is ODN-A151 (ODN151). In embodiments, the one or more cGAS-STING pathway inhibitors is Ru.521. In embodiments, the one or more cGAS-STING pathway inhibitors is G140.

In embodiments, the population of T cells is independently contacted with about 0.1 uM to about 50 uM of the one or more cGAS-STING pathway inhibitors. In embodiments, the population of T cells is independently contacted with about 1 uM to about 50 uM of the one or more cGAS-STING pathway inhibitors. In embodiments, the population of T cells is independently contacted with about 2 uM to about 50 uM of the one or more cGAS-STING pathway inhibitors. In embodiments, the population of T cells is independently contacted with about 3 uM to about 50 uM of the one or more cGAS-STING pathway inhibitors. In embodiments, the population of T cells is independently contacted with about 4 uM to about 50 uM of the one or more cGAS-STING pathway inhibitors. In embodiments, the population of T cells is independently contacted with about 5 uM to about 50 uM of the one or more cGAS-STING pathway inhibitors. In embodiments, the population of T cells is independently contacted with about 6 uM to about 50 uM of the one or more cGAS-STING pathway inhibitors. In embodiments, the population of T cells is independently contacted with about 7 uM to about 50 uM of the one or more cGAS-STING pathway inhibitors. In embodiments, the population of T cells is independently contacted with about 8 uM to about 50 uM of the one or more cGAS-STING pathway inhibitors. In embodiments, the population of T cells is independently contacted with about 9 uM to about 50 uM of the one or more cGAS-STING pathway inhibitors. In embodiments, the population of T cells is independently contacted with about 10 uM to about 50 uM of the one or more cGAS-STING pathway inhibitors. In embodiments, the population of T cells is independently contacted with about 11 uM to about 50 uM of the one or more cGAS-STING pathway inhibitors. In embodiments, the population of T cells is independently contacted with about 12 uM to about 50 uM of the one or more cGAS-STING pathway inhibitors. In embodiments, the population of T cells is independently contacted with about 13 uM to about 50 uM of the one or more cGAS-STING pathway inhibitors. In embodiments, the population of T cells is independently contacted with about 14 uM to about 50 uM of the one or more cGAS-STING pathway inhibitors. In embodiments, the population of T cells is independently contacted with about 15 uM to about 50 uM of the one or more cGAS-STING pathway inhibitors. In embodiments, the population of T cells is independently contacted with about 16 uM to about 50 uM of the one or more cGAS-STING pathway inhibitors. In embodiments, the population of T cells is independently contacted with about 17 uM to about 50 uM of the one or more cGAS-STING pathway inhibitors. In embodiments, the population of T cells is independently contacted with about 18 uM to about 50 uM of the one or more cGAS-STING pathway inhibitors. In embodiments, the population of T cells is independently contacted with about 19 uM to about 50 uM of the one or more cGAS-STING pathway inhibitors. In embodiments, the population of T cells is independently contacted with about 20 uM to about 50 uM of the one or more cGAS-STING pathway inhibitors. In embodiments, the population of T cells is independently contacted with about 21 uM to about 50 uM of the one or more cGAS-STING pathway inhibitors. In embodiments, the population of T cells is independently contacted with about 22 uM to about 50 uM of the one or more cGAS-STING pathway inhibitors. In embodiments, the population of T cells is independently contacted with about 23 uM to about 50 uM of the one or more cGAS-STING pathway inhibitors. In embodiments, the population of T cells is independently contacted with about 24 uM to about 50 uM of the one or more cGAS-STING pathway inhibitors. In embodiments, the population of T cells is independently contacted with about 25 uM to about 50 uM of the one or more cGAS-STING pathway inhibitors. In embodiments, the population of T cells is independently contacted with about 26 uM to about 50 uM of the one or more cGAS-STING pathway inhibitors. In embodiments, the population of T cells is independently contacted with about 27 uM to about 50 uM of the one or more cGAS-STING pathway inhibitors. In embodiments, the population of T cells is independently contacted with about 28 uM to about 50 uM of the one or more cGAS-STING pathway inhibitors. In embodiments, the population of T cells is independently contacted with about 29 uM to about 50 uM of the one or more cGAS-STING pathway inhibitors. In embodiments, the population of T cells is independently contacted with about 30 uM to about 50 uM of the one or more cGAS-STING pathway inhibitors. In embodiments, the population of T cells is independently contacted with about 31 uM to about 50 uM of the one or more cGAS-STING pathway inhibitors. In embodiments, the population of T cells is independently contacted with about 32 uM to about 50 uM of the one or more cGAS-STING pathway inhibitors. In embodiments, the population of T cells is independently contacted with about 33 uM to about 50 uM of the one or more cGAS-STING pathway inhibitors. In embodiments, the population of T cells is independently contacted with about 34 uM to about 50 uM of the one or more cGAS-STING pathway inhibitors. In embodiments, the population of T cells is independently contacted with about 35 uM to about 50 uM of the one or more cGAS-STING pathway inhibitors. In embodiments, the population of T cells is independently contacted with about 36 uM to about 50 uM of the one or more cGAS-STING pathway inhibitors. In embodiments, the population of T cells is independently contacted with about 37 uM to about 50 uM of the one or more cGAS-STING pathway inhibitors. In embodiments, the population of T cells is independently contacted with about 38 uM to about 50 uM of the one or more cGAS-STING pathway inhibitors. In embodiments, the population of T cells is independently contacted with about 39 uM to about 50 uM of the one or more cGAS-STING pathway inhibitors. In embodiments, the population of T cells is independently contacted with about 40 uM to about 50 uM of the one or more cGAS-STING pathway inhibitors. In embodiments, the population of T cells is independently contacted with about 41 uM to about 50 uM of the one or more cGAS-STING pathway inhibitors. In embodiments, the population of T cells is independently contacted with about 42 uM to about 50 uM of the one or more cGAS-STING pathway inhibitors. In embodiments, the population of T cells is independently contacted with about 43 uM to about 50 uM of the one or more cGAS-STING pathway inhibitors. In embodiments, the population of T cells is independently contacted with about 44 uM to about 50 uM of the one or more cGAS-STING pathway inhibitors. In embodiments, the population of T cells is independently contacted with about 45 uM to about 50 uM of the one or more cGAS-STING pathway inhibitors. In embodiments, the population of T cells is independently contacted with about 46 uM to about 50 uM of the one or more cGAS-STING pathway inhibitors. In embodiments, the population of T cells is independently contacted with about 47 uM to about 50 uM of the one or more cGAS-STING pathway inhibitors. In embodiments, the population of T cells is independently contacted with about 48 uM to about 50 uM of the one or more cGAS-STING pathway inhibitors. In embodiments, the population of T cells is independently contacted with about 49 uM to about 50 uM of the one or more cGAS-STING pathway inhibitors.

In embodiments, the population of T cells is independently contacted with about 0.1 uM to about 49 uM of the one or more cGAS-STING pathway inhibitors. In embodiments, the population of T cells is independently contacted with about 0.1 uM to about 48 uM of the one or more cGAS-STING pathway inhibitors. In embodiments, the population of T cells is independently contacted with about 0.1 uM to about 47 uM of the one or more cGAS-STING pathway inhibitors. In embodiments, the population of T cells is independently contacted with about 0.1 uM to about 46 uM of the one or more cGAS-STING pathway inhibitors. In embodiments, the population of T cells is independently contacted with about 0.1 uM to about 45 uM of the one or more cGAS-STING pathway inhibitors. In embodiments, the population of T cells is independently contacted with about 0.1 uM to about 44 uM of the one or more cGAS-STING pathway inhibitors. In embodiments, the population of T cells is independently contacted with about 0.1 uM to about 43 uM of the one or more cGAS-STING pathway inhibitors. In embodiments, the population of T cells is independently contacted with about 0.1 uM to about 42 uM of the one or more cGAS-STING pathway inhibitors. In embodiments, the population of T cells is independently contacted with about 0.1 uM to about 41 uM of the one or more cGAS-STING pathway inhibitors. In embodiments, the population of T cells is independently contacted with about 0.1 uM to about 40 uM of the one or more cGAS-STING pathway inhibitors. In embodiments, the population of T cells is independently contacted with about 0.1 uM to about 39 uM of the one or more cGAS-STING pathway inhibitors. In embodiments, the population of T cells is independently contacted with about 0.1 uM to about 38 uM of the one or more cGAS-STING pathway inhibitors. In embodiments, the population of T cells is independently contacted with about 0.1 uM to about 37 uM of the one or more cGAS-STING pathway inhibitors. In embodiments, the population of T cells is independently contacted with about 0.1 uM to about 36 uM of the one or more cGAS-STING pathway inhibitors. In embodiments, the population of T cells is independently contacted with about 0.1 uM to about 35 uM of the one or more cGAS-STING pathway inhibitors. In embodiments, the population of T cells is independently contacted with about 0.1 uM to about 34 uM of the one or more cGAS-STING pathway inhibitors. In embodiments, the population of T cells is independently contacted with about 0.1 uM to about 33 uM of the one or more cGAS-STING pathway inhibitors. In embodiments, the population of T cells is independently contacted with about 0.1 uM to about 32 uM of the one or more cGAS-STING pathway inhibitors. In embodiments, the population of T cells is independently contacted with about 0.1 uM to about 31 uM of the one or more cGAS-STING pathway inhibitors. In embodiments, the population of T cells is independently contacted with about 0.1 uM to about 30 uM of the one or more cGAS-STING pathway inhibitors. In embodiments, the population of T cells is independently contacted with about 0.1 uM to about 29 uM of the one or more cGAS-STING pathway inhibitors. In embodiments, the population of T cells is independently contacted with about 0.1 uM to about 28 uM of the one or more cGAS-STING pathway inhibitors. In embodiments, the population of T cells is independently contacted with about 0.1 uM to about 27 uM of the one or more cGAS-STING pathway inhibitors. In embodiments, the population of T cells is independently contacted with about 0.1 uM to about 26 uM of the one or more cGAS-STING pathway inhibitors. In embodiments, the population of T cells is independently contacted with about 0.1 uM to about 25 uM of the one or more cGAS-STING pathway inhibitors. In embodiments, the population of T cells is independently contacted with about 0.1 uM to about 24 uM of the one or more cGAS-STING pathway inhibitors. In embodiments, the population of T cells is independently contacted with about 0.1 uM to about 23 uM of the one or more cGAS-STING pathway inhibitors. In embodiments, the population of T cells is independently contacted with about 0.1 uM to about 22 uM of the one or more cGAS-STING pathway inhibitors. In embodiments, the population of T cells is independently contacted with about 0.1 uM to about 21 uM of the one or more cGAS-STING pathway inhibitors. In embodiments, the population of T cells is independently contacted with about 0.1 uM to about 20 uM of the one or more cGAS-STING pathway inhibitors. In embodiments, the population of T cells is independently contacted with about 0.1 uM to about 19 uM of the one or more cGAS-STING pathway inhibitors. In embodiments, the population of T cells is independently contacted with about 0.1 uM to about 18 uM of the one or more cGAS-STING pathway inhibitors. In embodiments, the population of T cells is independently contacted with about 0.1 uM to about 17 uM of the one or more cGAS-STING pathway inhibitors. In embodiments, the population of T cells is independently contacted with about 0.1 uM to about 16 uM of the one or more cGAS-STING pathway inhibitors. In embodiments, the population of T cells is independently contacted with about 0.1 uM to about 15 uM of the one or more cGAS-STING pathway inhibitors. In embodiments, the population of T cells is independently contacted with about 0.1 uM to about 14 uM of the one or more cGAS-STING pathway inhibitors. In embodiments, the population of T cells is independently contacted with about 0.1 uM to about 13 uM of the one or more cGAS-STING pathway inhibitors. In embodiments, the population of T cells is independently contacted with about 0.1 uM to about 12 uM of the one or more cGAS-STING pathway inhibitors. In embodiments, the population of T cells is independently contacted with about 0.1 uM to about 11 uM of the one or more cGAS-STING pathway inhibitors. In embodiments, the population of T cells is independently contacted with about 0.1 uM to about 10 uM of the one or more cGAS-STING pathway inhibitors. In embodiments, the population of T cells is independently contacted with about 0.1 uM to about 9 uM of the one or more cGAS-STING pathway inhibitors. In embodiments, the population of T cells is independently contacted with about 0.1 uM to about 8 uM of the one or more cGAS-STING pathway inhibitors. In embodiments, the population of T cells is independently contacted with about 0.1 uM to about 7 uM of the one or more cGAS-STING pathway inhibitors. In embodiments, the population of T cells is independently contacted with about 0.1 uM to about 6 uM of the one or more cGAS-STING pathway inhibitors. In embodiments, the population of T cells is independently contacted with about 0.1 uM to about 5 uM of the one or more cGAS-STING pathway inhibitors. In embodiments, the population of T cells is independently contacted with about 0.1 uM to about 4 uM of the one or more cGAS-STING pathway inhibitors. In embodiments, the population of T cells is independently contacted with about 0.1 uM to about 3 uM of the one or more cGAS-STING pathway inhibitors. In embodiments, the population of T cells is independently contacted with about 0.1 uM to about 2 uM of the one or more cGAS-STING pathway inhibitors. In embodiments, the population of T cells is independently contacted with about 0.1 uM to about 1 uM of the one or more cGAS-STING pathway inhibitors. In embodiments, the population of T cells is independently contacted with about 0.1 uM, 1 uM, 2 uM, 3 uM, 4 uM, 5 uM, 6 uM, 7 uM, 8 uM, 9 uM, 10 uM, 11 uM, 12 uM, 13 uM, 14 uM, 15 uM, 16 uM, 17 uM, 18 uM, 19 uM, 20 uM, 21 uM, 22 uM, 23 uM, 24 uM, 25 uM, 26 uM, 27 uM, 28 uM, 29 uM, 30 uM, 31 uM, 32 uM, 33 uM, 34 uM, 35 uM, 36 uM, 37 uM, 38 uM, 39 uM, 40 uM, 41 uM, 42 uM, 43 uM, 44 uM, 45 uM, 46 uM, 47 uM, 48 uM, 49 uM, or 50 uM of the one or more cGAS-STING pathway inhibitors.

In embodiments, the population of T cells is contacted with the nucleic acid in the presence of one or more cGAS-STING pathway inhibitors. In embodiments, the population of T cells is contacted sequentially with the nucleic acid and the one or more cGAS-STING pathway inhibitors. In embodiments, the population of T cells is contacted with the one or more cGAS-STING pathway inhibitors prior to the nucleic acid. In embodiments, the population of T cells is contacted with one cGAS-STING pathway inhibitor.

In embodiments, the population of T cells is contacted with about 1 uM to about 10 uM BX795. In embodiments, the population of T cells is contacted with about 1.5 uM to about 10 uM BX795. In embodiments, the population of T cells is contacted with about 2 uM to about 10 uM BX795. In embodiments, the population of T cells is contacted with about 2.5 uM to about 10 uM BX795. In embodiments, the population of T cells is contacted with about 3 uM to about 10 uM BX795. In embodiments, the population of T cells is contacted with about 3.5 uM to about 10 uM BX795. In embodiments, the population of T cells is contacted with about 4 uM to about 10 uM BX795. In embodiments, the population of T cells is contacted with about 4.5 uM to about 10 uM BX795. In embodiments, the population of T cells is contacted with about 5 uM to about 10 uM BX795. In embodiments, the population of T cells is contacted with about 5.5 uM to about 10 uM BX795. In embodiments, the population of T cells is contacted with about 6 uM to about 10 uM BX795. In embodiments, the population of T cells is contacted with about 6.5 uM to about 10 uM BX795. In embodiments, the population of T cells is contacted with about 7 uM to about 10 uM BX795. In embodiments, the population of T cells is contacted with about 7.5 uM to about 10 uM BX795. In embodiments, the population of T cells is contacted with about 8 uM to about 10 uM BX795. In embodiments, the population of T cells is contacted with about 8.5 uM to about 10 uM BX795. In embodiments, the population of T cells is contacted with about 9 uM to about 10 uM BX795. In embodiments, the population of T cells is contacted with about 9.5 uM to about 10 uM BX795.

In embodiments, the population of T cells is contacted with about 1 uM to about 9.5 uM BX795. In embodiments, the population of T cells is contacted with about 1 uM to about 9 uM BX795. In embodiments, the population of T cells is contacted with about 1 uM to about 8.5 uM BX795. In embodiments, the population of T cells is contacted with about 1 uM to about 8 uM BX795. In embodiments, the population of T cells is contacted with about 1 uM to about 7.5 uM BX795. In embodiments, the population of T cells is contacted with about 1 uM to about 7 uM BX795. In embodiments, the population of T cells is contacted with about 1 uM to about 6.5 uM BX795. In embodiments, the population of T cells is contacted with about 1 uM to about 6 uM BX795. In embodiments, the population of T cells is contacted with about 1 uM to about 5.5 uM BX795. In embodiments, the population of T cells is contacted with about 1 uM to about 5 uM BX795. In embodiments, the population of T cells is contacted with about 1 uM to about 4.5 uM BX795. In embodiments, the population of T cells is contacted with about 1 uM to about 4 uM BX795. In embodiments, the population of T cells is contacted with about 1 uM to about 3.5 uM BX795. In embodiments, the population of T cells is contacted with about 1 uM to about 3 uM BX795. In embodiments, the population of T cells is contacted with about 1 uM to about 2.5 uM BX795. In embodiments, the population of T cells is contacted with about 1 uM to about 2 uM BX795. In embodiments, the population of T cells is contacted with about 1 uM to about 1.5 uM BX795. In embodiments, the population of T cells is contacted with about 1 uM, 1.5 uM, 2 uM, 2.5 uM, 3 uM, 3.5 uM, 4 uM, 4.5 uM, 5 uM, 5.5 uM, 6 uM, 6.5 uM, 7 uM, 7.5 uM, 8 uM, 8.5 uM, 9 uM, 9.5 uM, or 10 uM BX795. In embodiments, the population of T cells is contacted with about 2.5 uM BX795. In embodiments, the population of T cells is contacted with about 5 uM BX795.

In embodiments, the population of T cells is contacted with about 0.1 uM to about 8 uM ODN151. In embodiments, the population of T cells is contacted with about 0.5 uM to about 8 uM ODN151. In embodiments, the population of T cells is contacted with about 1 uM to about 8 uM ODN151. In embodiments, the population of T cells is contacted with about 1.5 uM to about 8 uM ODN151. In embodiments, the population of T cells is contacted with about 2 uM to about 8 uM ODN151. In embodiments, the population of T cells is contacted with about 2.5 uM to about 8 uM ODN151. In embodiments, the population of T cells is contacted with about 3 uM to about 8 uM ODN151. In embodiments, the population of T cells is contacted with about 3.5 uM to about 8 uM ODN151. In embodiments, the population of T cells is contacted with about 4 uM to about 8 uM ODN151. In embodiments, the population of T cells is contacted with about 4.5 uM to about 8 uM ODN151. In embodiments, the population of T cells is contacted with about 5 uM to about 8 uM ODN151. In embodiments, the population of T cells is contacted with about 5.5 uM to about 8 uM ODN151. In embodiments, the population of T cells is contacted with about 6 uM to about 8 uM ODN151. In embodiments, the population of T cells is contacted with about 6.5 uM to about 8 uM ODN151. In embodiments, the population of T cells is contacted with about 7 uM to about 8 uM ODN151. In embodiments, the population of T cells is contacted with about 7.5 uM to about 8 uM ODN151.

In embodiments, the population of T cells is contacted with about 0.1 uM to about 7.5 uM ODN151. In embodiments, the population of T cells is contacted with about 0.1 uM to about 7 uM ODN151. In embodiments, the population of T cells is contacted with about 0.1 uM to about 6.5 uM ODN151. In embodiments, the population of T cells is contacted with about 0.1 uM to about 6 uM ODN151. In embodiments, the population of T cells is contacted with about 0.1 uM to about 5.5 uM ODN151. In embodiments, the population of T cells is contacted with about 0.1 uM to about 5 uM ODN151. In embodiments, the population of T cells is contacted with about 0.1 uM to about 4.5 uM ODN151. In embodiments, the population of T cells is contacted with about 0.1 uM to about 4 uM ODN151. In embodiments, the population of T cells is contacted with about 0.1 uM to about 3.5 uM ODN151. In embodiments, the population of T cells is contacted with about 0.1 uM to about 3 uM ODN151. In embodiments, the population of T cells is contacted with about 0.1 uM to about 2.5 uM ODN151. In embodiments, the population of T cells is contacted with about 0.1 uM to about 2 uM ODN151. In embodiments, the population of T cells is contacted with about 0.1 uM to about 1.5 uM ODN151. In embodiments, the population of T cells is contacted with about 0.1 uM to about 1 uM ODN151. In embodiments, the population of T cells is contacted with about 0.1 uM to about 0.5 uM ODN151. In embodiments, the population of T cells is contacted with about 0.1 uM, 0.5 uM, 1 uM, 1.5 uM, 2 uM, 2.5 uM, 3 uM, 3.5 uM, 4 uM, 4.5 uM, 5 uM, 5.5 uM, 6 uM, 6.5 uM, 7 uM, 7.5 uM, 8 uM, 8.5 uM, 9 uM, 9.5 uM, or 10 uM ODN151.

As described above, the methods provided herein including embodiments thereof are effective for increasing cell viability of engineered T cells compared to engineered T cells made without treatment with one or more cGAS-STING pathway inhibitors. For example, the population of engineered T cells as provided herein have increased cell viability compared to a population of engineered T cells made without contacting T cells with one or more cGAS-STING pathway inhibitors. In embodiments, the cell viability of the population of engineered T cells is increased from at least about 0.5 fold to at least about 5 fold relative to a population of engineered T cells that are not contacted with one or more cGAS-STING pathway inhibitors. In embodiments, the cell viability of the population of engineered T cells is increased from at least about 1 fold to at least about 5 fold relative to a population of engineered T cells that are not contacted with one or more cGAS-STING pathway inhibitors. In embodiments, the cell viability of the population of engineered T cells is increased from at least about 1.5 fold to at least about 5 fold relative to a population of engineered T cells that are not contacted with one or more cGAS-STING pathway inhibitors. In embodiments, the cell viability of the population of engineered T cells is increased from at least about 2 fold to at least about 5 fold relative to a population of engineered T cells that are not contacted with one or more cGAS-STING pathway inhibitors. In embodiments, the cell viability of the population of engineered T cells is increased from at least about 2.5 fold to at least about 5 fold relative to a population of engineered T cells that are not contacted with one or more cGAS-STING pathway inhibitors. In embodiments, the cell viability of the population of engineered T cells is increased from at least about 3 fold to at least about 5 fold relative to a population of engineered T cells that are not contacted with one or more cGAS-STING pathway inhibitors. In embodiments, the cell viability of the population of engineered T cells is increased from at least about 3.5 fold to at least about 5 fold relative to a population of engineered T cells that are not contacted with one or more cGAS-STING pathway inhibitors. In embodiments, the cell viability of the population of engineered T cells is increased from at least about 4 fold to at least about 5 fold relative to a population of engineered T cells that are not contacted with one or more cGAS-STING pathway inhibitors.

In embodiments, the cell viability of the population of engineered T cells is increased from at least about 0.5 fold to at least about 4.5 fold relative to a population of engineered T cells that are not contacted with one or more cGAS-STING pathway inhibitors. In embodiments, the cell viability of the population of engineered T cells is increased from at least about 0.5 fold to at least about 4 fold relative to a population of engineered T cells that are not contacted with one or more cGAS-STING pathway inhibitors. In embodiments, the cell viability of the population of engineered T cells is increased from at least about 0.5 fold to at least about 3.5 fold relative to a population of engineered T cells that are not contacted with one or more cGAS-STING pathway inhibitors. In embodiments, the cell viability of the population of engineered T cells is increased from at least about 0.5 fold to at least about 3 fold relative to a population of engineered T cells that are not contacted with one or more cGAS-STING pathway inhibitors. In embodiments, the cell viability of the population of engineered T cells is increased from at least about 0.5 fold to at least about 2.5 fold relative to a population of engineered T cells that are not contacted with one or more cGAS-STING pathway inhibitors. In embodiments, the cell viability of the population of engineered T cells is increased from at least about 0.5 fold to at least about 2 fold relative to a population of engineered T cells that are not contacted with one or more cGAS-STING pathway inhibitors. In embodiments, the cell viability of the population of engineered T cells is increased from at least about 0.5 fold to at least about 1.5 fold relative to a population of engineered T cells that are not contacted with one or more cGAS-STING pathway inhibitors. In embodiments, the cell viability of the population of engineered T cells is increased from at least about 0.5 fold to at least about 1 fold relative to a population of engineered T cells that are not contacted with one or more cGAS-STING pathway inhibitors. In embodiments, the cell viability of the population of engineered T cells is increased by about 0.5 fold, 1 fold, 1.5 fold, 2 fold, 2.5 fold, 3 fold, 3.5 fold, 4 fold, 4.5 fold, or 5 fold relative to a population of engineered T cells that are not contacted with one or more cGAS-STING pathway inhibitors. In embodiments, the cell viability is increased about 2 fold relative to a population of engineered T cells that are not contacted with one or more cGAS-STING pathway inhibitors. In embodiments, the cell viability is increased 2 fold relative to a population of engineered T cells that are not contacted with one or more cGAS-STING pathway inhibitors.

In embodiments, the cell viability of the population of engineered T cells is increased from about 30% to about 95% relative to a population of engineered T cells that are not contacted with one or more cGAS-STING pathway inhibitors. In embodiments, the cell viability of the population of engineered T cells is increased from about 35% to about 95% relative to a population of engineered T cells that are not contacted with one or more cGAS-STING pathway inhibitors. In embodiments, the cell viability of the population of engineered T cells is increased from about 40% to about 95% relative to a population of engineered T cells that are not contacted with one or more cGAS-STING pathway inhibitors. In embodiments, the cell viability of the population of engineered T cells is increased from about 45% to about 95% relative to a population of engineered T cells that are not contacted with one or more cGAS-STING pathway inhibitors. In embodiments, the cell viability of the population of engineered T cells is increased from about 50% to about 95% relative to a population of engineered T cells that are not contacted with one or more cGAS-STING pathway inhibitors. In embodiments, the cell viability of the population of engineered T cells is increased from about 55% to about 95% relative to a population of engineered T cells that are not contacted with one or more cGAS-STING pathway inhibitors. In embodiments, the cell viability of the population of engineered T cells is increased from about 60% to about 95% relative to a population of engineered T cells that are not contacted with one or more cGAS-STING pathway inhibitors. In embodiments, the cell viability of the population of engineered T cells is increased from about 65% to about 95% relative to a population of engineered T cells that are not contacted with one or more cGAS-STING pathway inhibitors. In embodiments, the cell viability of the population of engineered T cells is increased from about 70% to about 95% relative to a population of engineered T cells that are not contacted with one or more cGAS-STING pathway inhibitors. In embodiments, the cell viability of the population of engineered T cells is increased from about 75% to about 95% relative to a population of engineered T cells that are not contacted with one or more cGAS-STING pathway inhibitors. In embodiments, the cell viability of the population of engineered T cells is increased from about 80% to about 95% relative to a population of engineered T cells that are not contacted with one or more cGAS-STING pathway inhibitors. In embodiments, the cell viability of the population of engineered T cells is increased from about 85% to about 95% relative to a population of engineered T cells that are not contacted with one or more cGAS-STING pathway inhibitors. In embodiments, the cell viability of the population of engineered T cells is increased from about 90% to about 95% relative to a population of engineered T cells that are not contacted with one or more cGAS-STING pathway inhibitors.

In embodiments, the cell viability of the population of engineered T cells is increased from about 35% to about 90% relative to a population of engineered T cells that are not contacted with one or more cGAS-STING pathway inhibitors. In embodiments, the cell viability of the population of engineered T cells is increased from about 35% to about 85% relative to a population of engineered T cells that are not contacted with one or more cGAS-STING pathway inhibitors. In embodiments, the cell viability of the population of engineered T cells is increased from about 35% to about 80% relative to a population of engineered T cells that are not contacted with one or more cGAS-STING pathway inhibitors. In embodiments, the cell viability of the population of engineered T cells is increased from about 35% to about 75% relative to a population of engineered T cells that are not contacted with one or more cGAS-STING pathway inhibitors. In embodiments, the cell viability of the population of engineered T cells is increased from about 35% to about 70% relative to a population of engineered T cells that are not contacted with one or more cGAS-STING pathway inhibitors. In embodiments, the cell viability of the population of engineered T cells is increased from about 35% to about 65% relative to a population of engineered T cells that are not contacted with one or more cGAS-STING pathway inhibitors. In embodiments, the cell viability of the population of engineered T cells is increased from about 35% to about 60% relative to a population of engineered T cells that are not contacted with one or more cGAS-STING pathway inhibitors. In embodiments, the cell viability of the population of engineered T cells is increased from about 35% to about 55% relative to a population of engineered T cells that are not contacted with one or more cGAS-STING pathway inhibitors. In embodiments, the cell viability of the population of engineered T cells is increased from about 35% to about 50% relative to a population of engineered T cells that are not contacted with one or more cGAS-STING pathway inhibitors. In embodiments, the cell viability of the population of engineered T cells is increased from about 35% to about 45% relative to a population of engineered T cells that are not contacted with one or more cGAS-STING pathway inhibitors. In embodiments, the cell viability of the population of engineered T cells is increased from about 35% to about 40% relative to a population of engineered T cells that are not contacted with one or more cGAS-STING pathway inhibitors. In embodiments, the cell viability of the population of engineered T cells is increased about 35%, 40%, 45%, 50%, 55%, 60%, 65%, 70%, 75%, 80%, 85%, 90%, or 95% relative to a population of engineered T cells that are not contacted with one or more cGAS-STING pathway inhibitors.

The methods provided herein are effective for improving gene editing efficiency in T cells, thereby increasing the total number of engineered T cells. For example, the methods may increase efficiency of gene knock-out and/or gene knock-in, thereby improving gene editing efficiency of the T cells. The methods may thereby improve the yield of engineered T cells generated from a population of T cells. For example, the methods provided herein may generate an increased population of engineered T cells from a population of T cells relative to a population of engineered T cells wherein the T cells are not contacted with one or more cGAS-STING pathway inhibitors. Thus, in an aspect is provided a method of increasing gene editing efficiency in a population of T cells, including contacting a population of T cells with one or more cyclic GMP-AMP synthase (cGAS)-stimulator of interferon gene (STING) pathway inhibitors and a nucleic acid, thereby forming a population of engineered T cells, wherein the population of T cells has increased gene editing efficiency relative to a population of T cells that are not contacted with one or more cGAS-STING pathway inhibitors.

In embodiments, the nucleic acid includes a donor DNA. In embodiments, the nucleic acid is a plasmid, a nanoplasmid, a minicircle, or a viral vector including the donor DNA. In embodiments, the donor DNA encodes an exogenous T cell receptor (TCR)-beta or a fragment thereof, an exogenous TCR-alpha or a fragment thereof, or a combination thereof. In embodiments, the nucleic acid is associated with a delivery vehicle. In embodiments, the delivery vehicle is a lipid particle or a nanoparticle.

In embodiments, the population of T cells includes primary T cells.

In embodiments, the method further includes contacting the population of T cells with a gene editing reagent. In embodiments, contacting the population of T cells with the gene editing reagent includes contacting the population of T cells with a nucleic acid sequence encoding the gene editing reagent. In embodiments, the population of T cells is contacted with the nucleic acid in the presence of the gene editing agent or the nucleic acid sequence encoding the gene editing reagent. In embodiments, the gene editing reagent includes an RNA-guided nuclease. In embodiments, the RNA-guided nuclease is a CRISPR-Cas system.

In embodiments, the donor DNA is inserted into an endogenous TCR locus. In embodiments, the endogenous TCR locus is an endogenous TCR-alpha locus, an endogenous TCR-beta locus, or a combination thereof.

In embodiments, contacting the population of T cells with the gene editing reagent includes transfecting the population of T cells with the gene editing reagent. In embodiments, contacting the population of T cells with the nucleic acid includes transfecting the population of T cells with the nucleic acid.

In embodiments, the one or more cGAS-STING pathway inhibitors includes a cGAS inhibitor. In embodiments, the one or more cGAS-STING pathway inhibitors includes a STING inhibitor. In embodiments, the one or more cGAS-STING pathway inhibitors includes a TANK-binding kinase 1 (TBK1) inhibitor. In embodiments, the one or more cGAS-STING pathway inhibitors is Amlexanox (Aml), MRT67307 (MRT), BX795, H151, ODN-A151 (ODN151), Ru.521, G140, or combinations thereof. In embodiments, the one or more cGAS-STING pathway inhibitors includes Aml. In embodiments, the one or more cGAS-STING pathway inhibitors includes MRT. In embodiments, the one or more cGAS-STING pathway inhibitors includes BX795. In embodiments, the one or more cGAS-STING pathway inhibitors includes H151. In embodiments, the one or more cGAS-STING pathway inhibitors includes ODN151. In embodiments, the one or more cGAS-STING pathway inhibitors includes Ru.521. In embodiments, the one or more cGAS-STING pathway inhibitors includes G140.

In embodiments, the one or more cGAS-STING pathway inhibitors is Aml. In embodiments, the one or more cGAS-STING pathway inhibitors is MRT. In embodiments, the one or more cGAS-STING pathway inhibitors is BX795. In embodiments, the one or more cGAS-STING pathway inhibitors is H151. In embodiments, the one or more cGAS-STING pathway inhibitors is ODN151. In embodiments, the one or more cGAS-STING pathway inhibitors is Ru.521. In embodiments, the one or more cGAS-STING pathway inhibitors is G140. In embodiments, the cGAS-STING pathway inhibitor is Aml and does not include any other cGAS-STING pathway inhibitor. In embodiments, the cGAS-STING pathway inhibitor is MRT and does not include any other cGAS-STING pathway inhibitor. In embodiments, the cGAS-STING pathway inhibitor is BX795 and does not include any other cGAS-STING pathway inhibitor. In embodiments, the cGAS-STING pathway inhibitor is H151 and does not include any other cGAS-STING pathway inhibitor. In embodiments, the cGAS-STING pathway inhibitor is ODN151 and does not include any other cGAS-STING pathway inhibitor. In embodiments, the cGAS-STING pathway inhibitor is Ru.521 and does not include any other cGAS-STING pathway inhibitor. In embodiments, the cGAS-STING pathway inhibitor is G140 and does not include any other cGAS-STING pathway inhibitor.

In embodiments, the population of T cells is independently contacted with about 0.1 uM to about 50 uM of the one or more cGAS-STING pathway inhibitors. In embodiments, the population of T cells is independently contacted with about 2 uM to about 50 uM of the one or more cGAS-STING pathway inhibitors. In embodiments, the population of T cells is independently contacted with about 4 uM to about 50 uM of the one or more cGAS-STING pathway inhibitors. In embodiments, the population of T cells is independently contacted with about 6 uM to about 50 uM of the one or more cGAS-STING pathway inhibitors. In embodiments, the population of T cells is independently contacted with about 8 uM to about 50 uM of the one or more cGAS-STING pathway inhibitors. In embodiments, the population of T cells is independently contacted with about 10 uM to about 50 uM of the one or more cGAS-STING pathway inhibitors. In embodiments, the population of T cells is independently contacted with about 12 uM to about 50 uM of the one or more cGAS-STING pathway inhibitors. In embodiments, the population of T cells is independently contacted with about 14 uM to about 50 uM of the one or more cGAS-STING pathway inhibitors. In embodiments, the population of T cells is independently contacted with about 16 uM to about 50 uM of the one or more cGAS-STING pathway inhibitors. In embodiments, the population of T cells is independently contacted with about 18 uM to about 50 uM of the one or more cGAS-STING pathway inhibitors. In embodiments, the population of T cells is independently contacted with about 20 uM to about 50 uM of the one or more cGAS-STING pathway inhibitors. In embodiments, the population of T cells is independently contacted with about 22 uM to about 50 uM of the one or more cGAS-STING pathway inhibitors. In embodiments, the population of T cells is independently contacted with about 24 uM to about 50 uM of the one or more cGAS-STING pathway inhibitors. In embodiments, the population of T cells is independently contacted with about 26 uM to about 50 uM of the one or more cGAS-STING pathway inhibitors. In embodiments, the population of T cells is independently contacted with about 28 uM to about 50 uM of the one or more cGAS-STING pathway inhibitors. In embodiments, the population of T cells is independently contacted with about 30 uM to about 50 uM of the one or more cGAS-STING pathway inhibitors. In embodiments, the population of T cells is independently contacted with about 32 uM to about 50 uM of the one or more cGAS-STING pathway inhibitors. In embodiments, the population of T cells is independently contacted with about 34 uM to about 50 uM of the one or more cGAS-STING pathway inhibitors. In embodiments, the population of T cells is independently contacted with about 36 uM to about 50 uM of the one or more cGAS-STING pathway inhibitors. In embodiments, the population of T cells is independently contacted with about 38 uM to about 50 uM of the one or more cGAS-STING pathway inhibitors. In embodiments, the population of T cells is independently contacted with about 40 uM to about 50 uM of the one or more cGAS-STING pathway inhibitors. In embodiments, the population of T cells is independently contacted with about 42 uM to about 50 uM of the one or more cGAS-STING pathway inhibitors. In embodiments, the population of T cells is independently contacted with about 44 uM to about 50 uM of the one or more cGAS-STING pathway inhibitors. In embodiments, the population of T cells is independently contacted with about 46 uM to about 50 uM of the one or more cGAS-STING pathway inhibitors. In embodiments, the population of T cells is independently contacted with about 48 uM to about 50 uM of the one or more cGAS-STING pathway inhibitors.

In embodiments, the population of T cells is independently contacted with about 0.1 uM to about 48 uM of the one or more cGAS-STING pathway inhibitors. In embodiments, the population of T cells is independently contacted with about 0.1 uM to about 46 uM of the one or more cGAS-STING pathway inhibitors. In embodiments, the population of T cells is independently contacted with about 0.1 uM to about 44 uM of the one or more cGAS-STING pathway inhibitors. In embodiments, the population of T cells is independently contacted with about 0.1 uM to about 42 uM of the one or more cGAS-STING pathway inhibitors. In embodiments, the population of T cells is independently contacted with about 0.1 uM to about 40 uM of the one or more cGAS-STING pathway inhibitors. In embodiments, the population of T cells is independently contacted with about 0.1 uM to about 38 uM of the one or more cGAS-STING pathway inhibitors. In embodiments, the population of T cells is independently contacted with about 0.1 uM to about 36 uM of the one or more cGAS-STING pathway inhibitors. In embodiments, the population of T cells is independently contacted with about 0.1 uM to about 34 uM of the one or more cGAS-STING pathway inhibitors. In embodiments, the population of T cells is independently contacted with about 0.1 uM to about 32 uM of the one or more cGAS-STING pathway inhibitors. In embodiments, the population of T cells is independently contacted with about 0.1 uM to about 30 uM of the one or more cGAS-STING pathway inhibitors. In embodiments, the population of T cells is independently contacted with about 0.1 uM to about 28 uM of the one or more cGAS-STING pathway inhibitors. In embodiments, the population of T cells is independently contacted with about 0.1 uM to about 26 uM of the one or more cGAS-STING pathway inhibitors. In embodiments, the population of T cells is independently contacted with about 0.1 uM to about 24 uM of the one or more cGAS-STING pathway inhibitors. In embodiments, the population of T cells is independently contacted with about 0.1 uM to about 22 uM of the one or more cGAS-STING pathway inhibitors. In embodiments, the population of T cells is independently contacted with about 0.1 uM to about 20 uM of the one or more cGAS-STING pathway inhibitors. In embodiments, the population of T cells is independently contacted with about 0.1 uM to about 18 uM of the one or more cGAS-STING pathway inhibitors. In embodiments, the population of T cells is independently contacted with about 0.1 uM to about 16 uM of the one or more cGAS-STING pathway inhibitors. In embodiments, the population of T cells is independently contacted with about 0.1 uM to about 14 uM of the one or more cGAS-STING pathway inhibitors. In embodiments, the population of T cells is independently contacted with about 0.1 uM to about 12 uM of the one or more cGAS-STING pathway inhibitors. In embodiments, the population of T cells is independently contacted with about 0.1 uM to about 10 uM of the one or more cGAS-STING pathway inhibitors. In embodiments, the population of T cells is independently contacted with about 0.1 uM to about 8 uM of the one or more cGAS-STING pathway inhibitors. In embodiments, the population of T cells is independently contacted with about 0.1 uM to about 6 uM of the one or more cGAS-STING pathway inhibitors. In embodiments, the population of T cells is independently contacted with about 0.1 uM to about 4 uM of the one or more cGAS-STING pathway inhibitors. In embodiments, the population of T cells is independently contacted with about 0.1 uM to about 2 uM of the one or more cGAS-STING pathway inhibitors. In embodiments, the population of T cells is independently contacted with about 0.1 uM, 2 uM, 4 uM, 6 uM, 8 uM, 10 uM, 12 uM, 14 uM, 16 uM, 18 uM, 20 uM, 22 uM, 24 uM, 26 uM, 28 uM, 30 uM, 32 uM, 34 uM, 36 uM, 38 uM, 40 uM, 42 uM, 44 uM, 46 uM, 48 uM, or 50 uM of the one or more cGAS-STING pathway inhibitors.

In embodiments, the population of T cells is contacted with the nucleic acid in the presence of one or more cGAS-STING pathway inhibitors. In embodiments, the population of T cells is contacted sequentially with the nucleic acid and the one or more cGAS-STING pathway inhibitors. In embodiments, the population of T cells is contacted with the one or more cGAS-STING pathway inhibitors prior to the nucleic acid. In embodiments, the population of T cells is contacted with a cGAS-STING pathway inhibitor as provided herein and no other cGAS-STING pathway inhibitor.

In embodiments, the population of T cells is contacted with about 2 uM to about 8 uM BX795. In embodiments, the population of T cells is contacted with about 2.5 uM to about 8 uM BX795. In embodiments, the population of T cells is contacted with about 3 uM to about 8 uM BX795. In embodiments, the population of T cells is contacted with about 3.5 uM to about 8 uM BX795. In embodiments, the population of T cells is contacted with about 4 uM to about 8 uM BX795. In embodiments, the population of T cells is contacted with about 4.5 uM to about 8 uM BX795. In embodiments, the population of T cells is contacted with about 5 uM to about 8 uM BX795. In embodiments, the population of T cells is contacted with about 5.5 uM to about 8 uM BX795. In embodiments, the population of T cells is contacted with about 6 uM to about 8 uM BX795. In embodiments, the population of T cells is contacted with about 6.5 uM to about 8 uM BX795. In embodiments, the population of T cells is contacted with about 7 uM to about 8 uM BX795. In embodiments, the population of T cells is contacted with about 7.5 uM to about 8 uM BX795.

In embodiments, the population of T cells is contacted with about 2 uM to about 7.5 uM BX795. In embodiments, the population of T cells is contacted with about 2 uM to about 7 uM BX795. In embodiments, the population of T cells is contacted with about 2 uM to about 6.5 uM BX795. In embodiments, the population of T cells is contacted with about 2 uM to about 6 uM BX795. In embodiments, the population of T cells is contacted with about 2 uM to about 5.5 uM BX795. In embodiments, the population of T cells is contacted with about 2 uM to about 5 uM BX795. In embodiments, the population of T cells is contacted with about 2 uM to about 4.5 uM BX795. In embodiments, the population of T cells is contacted with about 2 uM to about 4 uM BX795. In embodiments, the population of T cells is contacted with about 2 uM to about 3.5 uM BX795. In embodiments, the population of T cells is contacted with about 2 uM to about 3 uM BX795. In embodiments, the population of T cells is contacted with about 2 uM to about 2.5 uM BX795. In embodiments, the population of T cells is contacted with about 2 uM, 2.5 uM, 3 uM, 3.5 uM, 4 uM, 4.5 uM, 5 uM, 5.5 uM, 6 uM, 6.5 uM, 7 uM, 7.5 uM, or 8 uM BX795.

In embodiments, the population of T cells is contacted with about 0.1 uM to about 5 uM ODNA151. In embodiments, the population of T cells is contacted with about 0.25 uM to about 5 uM ODNA151. In embodiments, the population of T cells is contacted with about 0.5 uM to about 5 uM ODNA151. In embodiments, the population of T cells is contacted with about 0.75 uM to about 5 uM ODNA151. In embodiments, the population of T cells is contacted with about 1 uM to about 5 uM ODNA151. In embodiments, the population of T cells is contacted with about 1.25 uM to about 5 uM ODNA151. In embodiments, the population of T cells is contacted with about 1.5 uM to about 5 uM ODNA151. In embodiments, the population of T cells is contacted with about 1.75 uM to about 5 uM ODNA151. In embodiments, the population of T cells is contacted with about 2 uM to about 5 uM ODNA151. In embodiments, the population of T cells is contacted with about 2.25 uM to about 5 uM ODNA151. In embodiments, the population of T cells is contacted with about 2.5 uM to about 5 uM ODNA151. In embodiments, the population of T cells is contacted with about 2.75 uM to about 5 uM ODNA151. In embodiments, the population of T cells is contacted with about 3 uM to about 5 uM ODNA151. In embodiments, the population of T cells is contacted with about 3.25 uM to about 5 uM ODNA151. In embodiments, the population of T cells is contacted with about 3.5 uM to about 5 uM ODNA151. In embodiments, the population of T cells is contacted with about 3.75 uM to about 5 uM ODNA151. In embodiments, the population of T cells is contacted with about 4 uM to about 5 uM ODNA151. In embodiments, the population of T cells is contacted with about 4.25 uM to about 5 uM ODNA151. In embodiments, the population of T cells is contacted with about 4.5 uM to about 5 uM ODNA151. In embodiments, the population of T cells is contacted with about 4.75 uM to about 5 uM ODNA151.

In embodiments, the population of T cells is contacted with about 0.1 uM to about 4.75 uM ODNA151. In embodiments, the population of T cells is contacted with about 0.1 uM to about 4.5 uM ODNA151. In embodiments, the population of T cells is contacted with about 0.1 uM to about 4.25 uM ODNA151. In embodiments, the population of T cells is contacted with about 0.1 uM to about 4 uM ODNA151. In embodiments, the population of T cells is contacted with about 0.1 uM to about 3.75 uM ODNA151. In embodiments, the population of T cells is contacted with about 0.1 uM to about 3.5 uM ODNA151. In embodiments, the population of T cells is contacted with about 0.1 uM to about 3.25 uM ODNA151. In embodiments, the population of T cells is contacted with about 0.1 uM to about 3 uM ODNA151. In embodiments, the population of T cells is contacted with about 0.1 uM to about 2.75 uM ODNA151. In embodiments, the population of T cells is contacted with about 0.1 uM to about 2.5 uM ODNA151. In embodiments, the population of T cells is contacted with about 0.1 uM to about 2.25 uM ODNA151. In embodiments, the population of T cells is contacted with about 0.1 uM to about 2 uM ODNA151. In embodiments, the population of T cells is contacted with about 0.1 uM to about 1.75 uM ODNA151. In embodiments, the population of T cells is contacted with about 0.1 uM to about 1.5 uM ODNA151. In embodiments, the population of T cells is contacted with about 0.1 uM to about 1.25 uM ODNA151. In embodiments, the population of T cells is contacted with about 0.1 uM to about 1 uM ODNA151. In embodiments, the population of T cells is contacted with about 0.1 uM to about 0.75 uM ODNA151. In embodiments, the population of T cells is contacted with about 0.1 uM to about 0.5 uM ODNA151. In embodiments, the population of T cells is contacted with about 0.1 uM to about 0.25 uM ODNA151. In embodiments, the population of T cells is contacted with about 0.1 uM, 0.25 uM, 0.5 uM, 0.75 uM, 1 uM, 1.25 uM, 1.5 uM, 1.75 uM, 2 uM, 2.25 uM, 2.5 uM, 2.75 uM, 3 uM, 3.25 uM, 3.5 uM, 3.75 uM, 4 uM, 4.25 uM, 4.5 uM, 4.75 uM, or 5 uM ODNA151.

In embodiments, the population of T cells is contacted with about 10 uM to about 50 uM Aml. In embodiments, the population of T cells is contacted with about 12 uM to about 50 uM Aml. In embodiments, the population of T cells is contacted with about 14 uM to about 50 uM Aml. In embodiments, the population of T cells is contacted with about 16 uM to about 50 uM Aml. In embodiments, the population of T cells is contacted with about 18 uM to about 50 uM Aml. In embodiments, the population of T cells is contacted with about 20 uM to about 50 uM Aml. In embodiments, the population of T cells is contacted with about 22 uM to about 50 uM Aml. In embodiments, the population of T cells is contacted with about 24 uM to about 50 uM Aml. In embodiments, the population of T cells is contacted with about 26 uM to about 50 uM Aml. In embodiments, the population of T cells is contacted with about 28 uM to about 50 uM Aml. In embodiments, the population of T cells is contacted with about 30 uM to about 50 uM Aml. In embodiments, the population of T cells is contacted with about 32 uM to about 50 uM Aml. In embodiments, the population of T cells is contacted with about 34 uM to about 50 uM Aml. In embodiments, the population of T cells is contacted with about 36 uM to about 50 uM Aml. In embodiments, the population of T cells is contacted with about 38 uM to about 50 uM Aml. In embodiments, the population of T cells is contacted with about 40 uM to about 50 uM Aml. In embodiments, the population of T cells is contacted with about 42 uM to about 50 uM Aml. In embodiments, the population of T cells is contacted with about 44 uM to about 50 uM Aml. In embodiments, the population of T cells is contacted with about 46 uM to about 50 uM Aml. In embodiments, the population of T cells is contacted with about 48 uM to about 50 uM Aml.

In embodiments, the population of T cells is contacted with about 10 uM to about 48 uM Aml. In embodiments, the population of T cells is contacted with about 10 uM to about 46 uM Aml. In embodiments, the population of T cells is contacted with about 10 uM to about 44 uM Aml. In embodiments, the population of T cells is contacted with about 10 uM to about 42 uM Aml. In embodiments, the population of T cells is contacted with about 10 uM to about 40 uM Aml. In embodiments, the population of T cells is contacted with about 10 uM to about 38 uM Aml. In embodiments, the population of T cells is contacted with about 10 uM to about 36 uM Aml. In embodiments, the population of T cells is contacted with about 10 uM to about 34 uM Aml. In embodiments, the population of T cells is contacted with about 10 uM to about 32 uM Aml. In embodiments, the population of T cells is contacted with about 10 uM to about 30 uM Aml. In embodiments, the population of T cells is contacted with about 10 uM to about 28 uM Aml. In embodiments, the population of T cells is contacted with about 10 uM to about 26 uM Aml. In embodiments, the population of T cells is contacted with about 10 uM to about 24 uM Aml. In embodiments, the population of T cells is contacted with about 10 uM to about 22 uM Aml. In embodiments, the population of T cells is contacted with about 10 uM to about 20 uM Aml. In embodiments, the population of T cells is contacted with about 10 uM to about 18 uM Aml. In embodiments, the population of T cells is contacted with about 10 uM to about 16 uM Aml. In embodiments, the population of T cells is contacted with about 10 uM to about 14 uM Aml. In embodiments, the population of T cells is contacted with about 10 uM to about 12 uM Aml. In embodiments, the population of T cells is contacted with about 10 uM, 12 uM, 14 uM, 16 uM, 18 uM, 20 uM, 22 uM, 24 uM, 26 uM, 28 uM, 30 uM, 32 uM, 34 uM, 36 uM, 38 uM, 40 uM, 42 uM, 44 uM, 46 uM, 48 uM, or 50 uM Aml.

In embodiments, the population of T cells is contacted with about 1 uM to about 10 uM MRT. In embodiments, the population of T cells is contacted with about 1.5 uM to about 10 uM MRT. In embodiments, the population of T cells is contacted with about 2 uM to about 10 uM MRT. In embodiments, the population of T cells is contacted with about 2.5 uM to about 10 uM MRT. In embodiments, the population of T cells is contacted with about 3 uM to about 10 uM MRT. In embodiments, the population of T cells is contacted with about 3.5 uM to about 10 uM MRT. In embodiments, the population of T cells is contacted with about 4 uM to about 10 uM MRT. In embodiments, the population of T cells is contacted with about 4.5 uM to about 10 uM MRT. In embodiments, the population of T cells is contacted with about 5 uM to about 10 uM MRT. In embodiments, the population of T cells is contacted with about 5.5 uM to about 10 uM MRT. In embodiments, the population of T cells is contacted with about 6 uM to about 10 uM MRT. In embodiments, the population of T cells is contacted with about 6.5 uM to about 10 uM MRT. In embodiments, the population of T cells is contacted with about 7 uM to about 10 uM MRT. In embodiments, the population of T cells is contacted with about 7.5 uM to about 10 uM MRT. In embodiments, the population of T cells is contacted with about 8 uM to about 10 uM MRT. In embodiments, the population of T cells is contacted with about 8.5 uM to about 10 uM MRT. In embodiments, the population of T cells is contacted with about 9 uM to about 10 uM MRT. In embodiments, the population of T cells is contacted with about 9.5 uM to about 10 uM MRT.

In embodiments, the population of T cells is contacted with about 1 uM to about 9.5 uM MRT. In embodiments, the population of T cells is contacted with about 1 uM to about 9 uM MRT. In embodiments, the population of T cells is contacted with about 1 uM to about 8.5 uM MRT. In embodiments, the population of T cells is contacted with about 1 uM to about 8 uM MRT. In embodiments, the population of T cells is contacted with about 1 uM to about 7.5 uM MRT. In embodiments, the population of T cells is contacted with about 1 uM to about 7 uM MRT. In embodiments, the population of T cells is contacted with about 1 uM to about 6.5 uM MRT. In embodiments, the population of T cells is contacted with about 1 uM to about 6 uM MRT. In embodiments, the population of T cells is contacted with about 1 uM to about 5.5 uM MRT. In embodiments, the population of T cells is contacted with about 1 uM to about 5 uM MRT. In embodiments, the population of T cells is contacted with about 1 uM to about 4.5 uM MRT. In embodiments, the population of T cells is contacted with about 1 uM to about 4 uM MRT. In embodiments, the population of T cells is contacted with about 1 uM to about 3.5 uM MRT. In embodiments, the population of T cells is contacted with about 1 uM to about 3 uM MRT. In embodiments, the population of T cells is contacted with about 1 uM to about 2.5 uM MRT. In embodiments, the population of T cells is contacted with about 1 uM to about 2 uM MRT. In embodiments, the population of T cells is contacted with about 1 uM to about 1.5 uM MRT. In embodiments, the population of T cells is contacted with about 1 uM, 1.5 uM, 2 uM, 2.5 uM, 3 uM, 3.5 uM, 4 uM, 4.5 uM, 5 uM, 5.5 uM, 6 uM, 6.5 uM, 7 uM, 7.5 uM, 8 uM, 8.5 uM, 9 uM, 9.5 uM, or 10 uM MRT.

In embodiments, the gene editing efficiency of the population of T cells is increased from at least about 0.5 fold to at least about 5 fold relative to a population of T cells that are not contacted with one or more cGAS-STING pathway inhibitors. In embodiments, the gene editing efficiency of the population of T cells is increased from at least about 1 fold to at least about 5 fold relative to a population of T cells that are not contacted with one or more cGAS-STING pathway inhibitors. In embodiments, the gene editing efficiency of the population of T cells is increased from at least about 1.5 fold to at least about 5 fold relative to a population of T cells that are not contacted with one or more cGAS-STING pathway inhibitors. In embodiments, the gene editing efficiency of the population of T cells is increased from at least about 2 fold to at least about 5 fold relative to a population of T cells that are not contacted with one or more cGAS-STING pathway inhibitors. In embodiments, the gene editing efficiency of the population of T cells is increased from at least about 2.5 fold to at least about 5 fold relative to a population of T cells that are not contacted with one or more cGAS-STING pathway inhibitors. In embodiments, the gene editing efficiency of the population of T cells is increased from at least about 3 fold to at least about 5 fold relative to a population of T cells that are not contacted with one or more cGAS-STING pathway inhibitors. In embodiments, the gene editing efficiency of the population of T cells is increased from at least about 3.5 fold to at least about 4 fold relative to a population of T cells that are not contacted with one or more cGAS-STING pathway inhibitors. In embodiments, the gene editing efficiency of the population of T cells is increased from at least about 4 fold to at least about 5 fold relative to a population of T cells that are not contacted with one or more cGAS-STING pathway inhibitors. In embodiments, the gene editing efficiency of the population of T cells is increased from at least about 4.5 fold to at least about 5 fold relative to a population of T cells that are not contacted with one or more cGAS-STING pathway inhibitors.

In embodiments, the gene editing efficiency of the population of T cells is increased from at least about 0.5 fold to at least about 4.5 fold relative to a population of T cells that are not contacted with one or more cGAS-STING pathway inhibitors. In embodiments, the gene editing efficiency of the population of T cells is increased from at least about 0.5 fold to at least about 4 fold relative to a population of T cells that are not contacted with one or more cGAS-STING pathway inhibitors. In embodiments, the gene editing efficiency of the population of T cells is increased from at least about 0.5 fold to at least about 3.5 fold relative to a population of T cells that are not contacted with one or more cGAS-STING pathway inhibitors. In embodiments, the gene editing efficiency of the population of T cells is increased from at least about 0.5 fold to at least about 3 fold relative to a population of T cells that are not contacted with one or more cGAS-STING pathway inhibitors. In embodiments, the gene editing efficiency of the population of T cells is increased from at least about 0.5 fold to at least about 2.5 fold relative to a population of T cells that are not contacted with one or more cGAS-STING pathway inhibitors. In embodiments, the gene editing efficiency of the population of T cells is increased from at least about 0.5 fold to at least about 2 fold relative to a population of T cells that are not contacted with one or more cGAS-STING pathway inhibitors. In embodiments, the gene editing efficiency of the population of T cells is increased from at least about 0.5 fold to at least about 1.5 fold relative to a population of T cells that are not contacted with one or more cGAS-STING pathway inhibitors. In embodiments, the gene editing efficiency of the population of T cells is increased from at least about 0.5 fold to at least about 1 fold relative to a population of T cells that are not contacted with one or more cGAS-STING pathway inhibitors. In embodiments, the gene editing efficiency of the population of T cells is increased at least about 0.5 fold, 1 fold, 1.5 fold, 2 fold, 2.5 fold, 3 fold, 3.5 fold, 4 fold, 4.5 fold, or 5 fold relative to a population of T cells that are not contacted with one or more cGAS-STING pathway inhibitors.

In embodiments, the gene editing efficiency of the population of T cells is increased from about 2 fold to about 3 fold relative to a population of T cells that are not contacted with one or more cGAS-STING pathway inhibitors. In embodiments, the gene editing efficiency of the population of T cells is increased from about 2.2 fold to about 3 fold relative to a population of T cells that are not contacted with one or more cGAS-STING pathway inhibitors. In embodiments, the gene editing efficiency of the population of T cells is increased from about 2.4 fold to about 3 fold relative to a population of T cells that are not contacted with one or more cGAS-STING pathway inhibitors. In embodiments, the gene editing efficiency of the population of T cells is increased from about 2.6 fold to about 3 fold relative to a population of T cells that are not contacted with one or more cGAS-STING pathway inhibitors. In embodiments, the gene editing efficiency of the population of T cells is increased from about 2.8 fold to about 3 fold relative to a population of T cells that are not contacted with one or more cGAS-STING pathway inhibitors.

In embodiments, the gene editing efficiency of the population of T cells is increased from about 2 fold to about 2.8 fold relative to a population of T cells that are not contacted with one or more cGAS-STING pathway inhibitors. In embodiments, the gene editing efficiency of the population of T cells is increased from about 2 fold to about 2.6 fold relative to a population of T cells that are not contacted with one or more cGAS-STING pathway inhibitors. In embodiments, the gene editing efficiency of the population of T cells is increased from about 2 fold to about 2.4 fold relative to a population of T cells that are not contacted with one or more cGAS-STING pathway inhibitors. In embodiments, the gene editing efficiency of the population of T cells is increased from about 2 fold to about 2.2 fold relative to a population of T cells that are not contacted with one or more cGAS-STING pathway inhibitors. In embodiments, the gene editing efficiency of the population of T cells is increased about 2 fold, 2.2 fold, 2.4 fold, 2.6 fold, 2.8 fold, or 3 fold relative to a population of T cells that are not contacted with one or more cGAS-STING pathway inhibitors.

In embodiments, the gene editing efficiency of the population of T cells is increased from about 60% to about 99% relative to a population of T cells that are not contacted with one or more cGAS-STING pathway inhibitors. In embodiments, the gene editing efficiency of the population of T cells is increased from about 63% to about 99% relative to a population of T cells that are not contacted with one or more cGAS-STING pathway inhibitors. In embodiments, the gene editing efficiency of the population of T cells is increased from about 66% to about 99% relative to a population of T cells that are not contacted with one or more cGAS-STING pathway inhibitors. In embodiments, the gene editing efficiency of the population of T cells is increased from about 69% to about 99% relative to a population of T cells that are not contacted with one or more cGAS-STING pathway inhibitors. In embodiments, the gene editing efficiency of the population of T cells is increased from about 72% to about 99% relative to a population of T cells that are not contacted with one or more cGAS-STING pathway inhibitors. In embodiments, the gene editing efficiency of the population of T cells is increased from about 75% to about 99% relative to a population of T cells that are not contacted with one or more cGAS-STING pathway inhibitors. In embodiments, the gene editing efficiency of the population of T cells is increased from about 78% to about 99% relative to a population of T cells that are not contacted with one or more cGAS-STING pathway inhibitors. In embodiments, the gene editing efficiency of the population of T cells is increased from about 81% to about 99% relative to a population of T cells that are not contacted with one or more cGAS-STING pathway inhibitors. In embodiments, the gene editing efficiency of the population of T cells is increased from about 84% to about 99% relative to a population of T cells that are not contacted with one or more cGAS-STING pathway inhibitors. In embodiments, knock-out efficiency of the population of T cells is increased from about 87% to about 99% relative to a population of T cells that are not contacted with one or more cGAS-STING pathway inhibitors. In embodiments, the gene editing efficiency of the population of T cells is increased from about 90% to about 99% relative to a population of T cells that are not contacted with one or more cGAS-STING pathway inhibitors. In embodiments, the gene editing efficiency of the population of T cells is increased from about 93% to about 99% relative to a population of T cells that are not contacted with one or more cGAS-STING pathway inhibitors. In embodiments, the gene editing efficiency of the population of T cells is increased from about 96% to about 99% relative to a population of T cells that are not contacted with one or more cGAS-STING pathway inhibitors.

In embodiments, the gene editing efficiency of the population of T cells is increased from about 60% to about 96% relative to a population of T cells that are not contacted with one or more cGAS-STING pathway inhibitors. In embodiments, the gene editing efficiency of the population of T cells is increased from about 60% to about 93% relative to a population of T cells that are not contacted with one or more cGAS-STING pathway inhibitors. In embodiments, the gene editing efficiency of the population of T cells is increased from about 60% to about 90% relative to a population of T cells that are not contacted with one or more cGAS-STING pathway inhibitors. In embodiments, the gene editing efficiency of the population of T cells is increased from about 60% to about 87% relative to a population of T cells that are not contacted with one or more cGAS-STING pathway inhibitors. In embodiments, the gene editing efficiency of the population of T cells is increased from about 60% to about 84% relative to a population of T cells that are not contacted with one or more cGAS-STING pathway inhibitors. In embodiments, the gene editing efficiency of the population of T cells is increased from about 60% to about 81% relative to a population of T cells that are not contacted with one or more cGAS-STING pathway inhibitors. In embodiments, the gene editing efficiency of the population of T cells is increased from about 60% to about 78% relative to a population of T cells that are not contacted with one or more cGAS-STING pathway inhibitors. In embodiments, the gene editing efficiency of the population of T cells is increased from about 60% to about 75% relative to a population of T cells that are not contacted with one or more cGAS-STING pathway inhibitors. In embodiments, the gene editing efficiency of the population of T cells is increased from about 60% to about 72% relative to a population of T cells that are not contacted with one or more cGAS-STING pathway inhibitors. In embodiments, the gene editing efficiency of the population of T cells is increased from about 60% to about 69% relative to a population of T cells that are not contacted with one or more cGAS-STING pathway inhibitors. In embodiments, the gene editing efficiency of the population of T cells is increased from about 60% to about 66% relative to a population of T cells that are not contacted with one or more cGAS-STING pathway inhibitors. In embodiments, the gene editing efficiency of the population of T cells is increased from about 60% to about 63% relative to a population of T cells that are not contacted with one or more cGAS-STING pathway inhibitors. In embodiments, the gene editing efficiency of the population of T cells is increased about 60%, 63%, 66%, 69%, 72%, 75%, 78%, 81%, 84%, 87%, 90%, 93%, 96%, or 99% relative to a population of T cells that are not contacted with one or more cGAS-STING pathway inhibitors.

In embodiments, the knock-out efficiency of the population of engineered T cells is from about 20% to about 100%. In embodiments, the knock-out efficiency of the population of engineered T cells is from about 30% to about 100%. In embodiments, the knock-out efficiency of the population of engineered T cells is from about 40% to about 100%. In embodiments, the knock-out efficiency of the population of engineered T cells is from about 50% to about 100%. In embodiments, the knock-out efficiency of the population of engineered T cells is from about 60% to about 100%. In embodiments, the knock-out efficiency of the population of engineered T cells is from about 70% to about 100%. In embodiments, the knock-out efficiency of the population of engineered T cells is from about 70% to about 99%. In embodiments, the knock-out efficiency of the population of engineered T cells is from about 72% to about 99%. In embodiments, the knock-out efficiency of the population of engineered T cells is from about 74% to about 99%. In embodiments, the knock-out efficiency of the population of engineered T cells is from about 76% to about 99%. In embodiments, the knock-out efficiency of the population of engineered T cells is from about 78% to about 99%. In embodiments, the knock-out efficiency of the population of engineered T cells is from about 80% to about 99%. In embodiments, the knock-out efficiency of the population of engineered T cells is from about 82% to about 99%. In embodiments, the knock-out efficiency of the population of engineered T cells is from about 84% to about 99%. In embodiments, the knock-out efficiency of the population of engineered T cells is from about 86% to about 99%. In embodiments, the knock-out efficiency of the population of engineered T cells is from about 88% to about 99%. In embodiments, the knock-out efficiency of the population of engineered T cells is from about 90% to about 99%. In embodiments, the knock-out efficiency of the population of engineered T cells is from about 92% to about 99%. In embodiments, the knock-out efficiency of the population of engineered T cells is from about 94% to about 99%. In embodiments, the knock-out efficiency of the population of engineered T cells is from about 96% to about 99%. In embodiments, the knock-out efficiency of the population of engineered T cells is from about 98% to about 99%.

In embodiments, the knock-out efficiency of the population of engineered T cells is from about 50% to about 98%. In embodiments, the knock-out efficiency of the population of engineered T cells is from about 60% to about 98%. In embodiments, the knock-out efficiency of the population of engineered T cells is from about 70% to about 98%. In embodiments, the knock-out efficiency of the population of engineered T cells is from about 70% to about 96%. In embodiments, the knock-out efficiency of the population of engineered T cells is from about 70% to about 94%. In embodiments, the knock-out efficiency of the population of engineered T cells is from about 70% to about 92%. In embodiments, the knock-out efficiency of the population of engineered T cells is from about 70% to about 90%. In embodiments, the knock-out efficiency of the population of engineered T cells is from about 70% to about 88%. In embodiments, the knock-out efficiency of the population of engineered T cells is from about 70% to about 86%. In embodiments, the knock-out efficiency of the population of engineered T cells is from about 70% to about 84%. In embodiments, the knock-out efficiency of the population of engineered T cells is from about 70% to about 82%. In embodiments, the knock-out efficiency of the population of engineered T cells is from about 70% to about 80%. In embodiments, the knock-out efficiency of the population of engineered T cells is from about 70% to about 78%. In embodiments, the knock-out efficiency of the population of engineered T cells is from about 70% to about 76%. In embodiments, the knock-out efficiency of the population of engineered T cells is from about 70% to about 74%. In embodiments, the knock-out efficiency of the population of engineered T cells is from about 70% to about 72%. In embodiments, the knock-out efficiency of the population of engineered T cells is about 20%, 30%, 40$, 50%, 55%, 60%, 65%, 70%, 72%, 74%, 76%, 78%, 80%, 82%, 84%, 86%, 88%, 90%, 92%, 94%, 96%, 98%, 99%, or about 100%. For the methods provided herein, in embodiments, the knock-out efficiency of the population of T cells is about 90%. In embodiments, the knock-out efficiency of the population of T cells is 90%.

In embodiments, knock-in efficiency of the population of T cells is from about 20% to about 100%. In embodiments, knock-in efficiency of the population of T cells is from about 20% to about 99%. In embodiments, knock-in efficiency of the population of T cells is from about 25% to about 99%. In embodiments, knock-in efficiency of the population of T cells is from about 30% to about 99%. In embodiments, knock-in efficiency of the population of T cells is from about 35% to about 99%. In embodiments, knock-in efficiency of the population of T cells is from about 40% to about 99%. In embodiments, knock-in efficiency of the population of T cells is from about 45% to about 99%. In embodiments, knock-in efficiency of the population of T cells is from about 50% to about 99%. In embodiments, knock-in efficiency of the population of T cells is from about 55% to about 99%. In embodiments, knock-in efficiency of the population of T cells is from about 60% to about 99%. In embodiments, knock-in efficiency of the population of T cells is from about 65% to about 99%. In embodiments, knock-in efficiency of the population of T cells is from about 70% to about 99%. In embodiments, knock-in efficiency of the population of T cells is from about 75% to about 99%. In embodiments, knock-in efficiency of the population of T cells is from about 80% to about 99%. In embodiments, knock-in efficiency of the population of T cells is from about 85% to about 99%. In embodiments, knock-in efficiency of the population of T cells is from about 90% to about 99%. In embodiments, knock-in efficiency of the population of T cells is from about 95% to about 99%.

In embodiments, knock-in efficiency of the population of T cells is from about 20% to about 95%. In embodiments, knock-in efficiency of the population of T cells is from about 20% to about 90%. In embodiments, knock-in efficiency of the population of T cells is from about 20% to about 85%. In embodiments, knock-in efficiency of the population of T cells is from about 20% to about 80%. In embodiments, knock-in efficiency of the population of T cells is from about 20% to about 75%. In embodiments, knock-in efficiency of the population of T cells is from about 20% to about 70%. In embodiments, knock-in efficiency of the population of T cells is from about 20% to about 65%. In embodiments, knock-in efficiency of the population of T cells is from about 20% to about 60%. In embodiments, knock-in efficiency of the population of T cells is from about 20% to about 55%. In embodiments, knock-in efficiency of the population of T cells is from about 20% to about 50%. In embodiments, knock-in efficiency of the population of T cells is from about 20% to about 45%. In embodiments, knock-in efficiency of the population of T cells is from about 20% to about 40%. In embodiments, knock-in efficiency of the population of T cells is from about 20% to about 35%. In embodiments, knock-in efficiency of the population of T cells is from about 20% to about 30%. In embodiments, knock-in efficiency of the population of T cells is from about 20% to about 25%. In embodiments, knock-in efficiency of the population of T cells is about 20%, 25%, 30%, 35%, 40%, 45%, 50%, 55%, 60%, 65%, 70%, 75%, 80%, 85%, 90%, 95%, 99%, or 100%. In embodiments, the knock-in efficiency is about 60%. In embodiments, the knock-in efficiency is 60%.

Provided herein, inter alia, are methods for increasing expansion of engineered T cells. The methods provided herein are contemplated to be effective for improving expansion of engineered T cells thereby increasing the total number of engineered T cells in a culture. Expansion of the engineered T cells is improved relative to engineered T cells generated without contact with one or more cGAS-STING pathway inhibitors. Thus, in an aspect is provided a method for increasing expansion of a population of engineered T cells, including i) contacting a population of T cells with one or more cyclic GMP-AMP synthase (cGAS)-stimulator of interferon gene (STING) pathway inhibitors and a nucleic acid, thereby forming the population of engineered T cells, and ii) expanding the population of engineered T cells, thereby forming a population of expanded engineered T cells, wherein the one or more cGAS-STING pathway inhibitors increases the population of expanded engineered T cells relative to a population of engineered T cells, wherein the population of T cells of step i) are not contacted with one or more cGAS-STING pathway inhibitors.

In embodiments, the nucleic acid includes a donor DNA. In embodiments, the nucleic acid is a plasmid, a nanoplasmid, a minicircle, or a viral vector comprising a donor DNA. In embodiments, the donor DNA encodes an exogenous T cell receptor (TCR)-beta or a fragment thereof, an exogenous TCR-alpha or a fragment thereof, or a combination thereof. In embodiments, the nucleic acid is associated with a delivery vehicle. In embodiments, the delivery vehicle is a lipid particle or a nanoparticle.

In embodiments, the population of T cells includes primary T cells.

For the methods provided herein, in embodiments, step i) further includes contacting the T cell with a gene editing reagent. In embodiments, contacting the T cell with the gene editing reagent includes contacting the T cell with a nucleic acid sequence encoding the gene editing reagent. In embodiments, the T cell is contacted with the nucleic acid in the presence of the gene editing agent or the nucleic acid sequence encoding the gene editing reagent.

In embodiments, the donor DNA is inserted into an endogenous TCR locus. In embodiments, the endogenous TCR locus is an endogenous TCR-alpha locus, an endogenous TCR-beta locus, or a combination thereof.

In embodiments, contacting the T cell with the gene editing reagent includes transfecting the T cell with the gene editing reagent. In embodiments, contacting the T cell with the nucleic acid includes transfecting the T cell with the nucleic acid.

In embodiments, the one or more cGAS-STING pathway inhibitors includes a cGAS inhibitor. In embodiments, the one or more cGAS-STING pathway inhibitors includes a TANK-binding kinase 1 (TBK1) inhibitor. In embodiments, the one or more cGAS-STING pathway inhibitors is Amlexanox (Aml), MRT67307 (MRT), BX795, H151, ODN-A151 (ODN151), Ru.521, G140, or combinations thereof. In embodiments, the one or more cGAS-STING pathway inhibitors includes Aml. In embodiments, the one or more cGAS-STING pathway inhibitors includes MRT. In embodiments, the one or more cGAS-STING pathway inhibitors includes BX795. In embodiments, the one or more cGAS-STING pathway inhibitors includes H151. In embodiments, the one or more cGAS-STING pathway inhibitors includes ODN-A151. In embodiments, the one or more cGAS-STING pathway inhibitors includes ODN151 In embodiments, the one or more cGAS-STING pathway inhibitors includes Ru.521. In embodiments, the one or more cGAS-STING pathway inhibitors includes G140.

In embodiments, the cGAS-STING pathway inhibitor is Aml. In embodiments, the cGAS-STING pathway inhibitor is MRT. In embodiments, the cGAS-STING pathway inhibitor is BX795. In embodiments, the cGAS-STING pathway inhibitor is H151. In embodiments, the cGAS-STING pathway inhibitor is ODN-A151. In embodiments, the cGAS-STING pathway inhibitor is ODN151 In embodiments, the cGAS-STING pathway inhibitor is Ru.521. In embodiments, the cGAS-STING pathway inhibitor is G140. In embodiments, the cGAS-STING pathway inhibitor is Aml and no other cGAS-STING pathway inhibitor. In embodiments, the cGAS-STING pathway inhibitor is MRT and no other cGAS-STING pathway inhibitor. In embodiments, the cGAS-STING pathway inhibitor is BX795 and no other cGAS-STING pathway inhibitor. In embodiments, the cGAS-STING pathway inhibitor is H151 and no other cGAS-STING pathway inhibitor. In embodiments, the cGAS-STING pathway inhibitor is ODN-A151 and no other cGAS-STING pathway inhibitor. In embodiments, the cGAS-STING pathway inhibitor is ODN151 and no other cGAS-STING pathway inhibitor. In embodiments, the cGAS-STING pathway inhibitor is Ru.521 and no other cGAS-STING pathway inhibitor. In embodiments, the cGAS-STING pathway inhibitor is G140 and no other cGAS-STING pathway inhibitor.

In embodiments, the population of T cells is independently contacted with about 1 uM to about 50 uM of the one or more cGAS-STING pathway inhibitors. In embodiments, the population of T cells is independently contacted with about 2 uM to about 50 uM of the one or more cGAS-STING pathway inhibitors. In embodiments, the population of T cells is independently contacted with about 4 uM to about 50 uM of the one or more cGAS-STING pathway inhibitors. In embodiments, the population of T cells is independently contacted with about 6 uM to about 50 uM of the one or more cGAS-STING pathway inhibitors. In embodiments, the population of T cells is independently contacted with about 8 uM to about 50 uM of the one or more cGAS-STING pathway inhibitors. In embodiments, the population of T cells is independently contacted with about 10 uM to about 50 uM of the one or more cGAS-STING pathway inhibitors. In embodiments, the population of T cells is independently contacted with about 12 uM to about 50 uM of the one or more cGAS-STING pathway inhibitors. In embodiments, the population of T cells is independently contacted with about 14 uM to about 50 uM of the one or more cGAS-STING pathway inhibitors. In embodiments, the population of T cells is independently contacted with about 16 uM to about 50 uM of the one or more cGAS-STING pathway inhibitors. In embodiments, the population of T cells is independently contacted with about 18 uM to about 50 uM of the one or more cGAS-STING pathway inhibitors. In embodiments, the population of T cells is independently contacted with about 20 uM to about 50 uM of the one or more cGAS-STING pathway inhibitors. In embodiments, the population of T cells is independently contacted with about 22 uM to about 50 uM of the one or more cGAS-STING pathway inhibitors. In embodiments, the population of T cells is independently contacted with about 24 uM to about 50 uM of the one or more cGAS-STING pathway inhibitors. In embodiments, the population of T cells is independently contacted with about 26 uM to about 50 uM of the one or more cGAS-STING pathway inhibitors. In embodiments, the population of T cells is independently contacted with about 28 uM to about 50 uM of the one or more cGAS-STING pathway inhibitors. In embodiments, the population of T cells is independently contacted with about 30 uM to about 50 uM of the one or more cGAS-STING pathway inhibitors. In embodiments, the population of T cells is independently contacted with about 32 uM to about 50 uM of the one or more cGAS-STING pathway inhibitors. In embodiments, the population of T cells is independently contacted with about 34 uM to about 50 uM of the one or more cGAS-STING pathway inhibitors. In embodiments, the population of T cells is independently contacted with about 36 uM to about 50 uM of the one or more cGAS-STING pathway inhibitors. In embodiments, the population of T cells is independently contacted with about 38 uM to about 50 uM of the one or more cGAS-STING pathway inhibitors. In embodiments, the population of T cells is independently contacted with about 40 uM to about 50 uM of the one or more cGAS-STING pathway inhibitors. In embodiments, the population of T cells is independently contacted with about 42 uM to about 50 uM of the one or more cGAS-STING pathway inhibitors. In embodiments, the population of T cells is independently contacted with about 44 uM to about 50 uM of the one or more cGAS-STING pathway inhibitors. In embodiments, the population of T cells is independently contacted with about 46 uM to about 50 uM of the one or more cGAS-STING pathway inhibitors. In embodiments, the population of T cells is independently contacted with about 48 uM to about 50 uM of the one or more cGAS-STING pathway inhibitors.

In embodiments, the population of T cells is independently contacted with about 1 uM to about 48 uM of the one or more cGAS-STING pathway inhibitors. In embodiments, the population of T cells is independently contacted with about 1 uM to about 46 uM of the one or more cGAS-STING pathway inhibitors. In embodiments, the population of T cells is independently contacted with about 1 uM to about 44 uM of the one or more cGAS-STING pathway inhibitors. In embodiments, the population of T cells is independently contacted with about 1 uM to about 42 uM of the one or more cGAS-STING pathway inhibitors. In embodiments, the population of T cells is independently contacted with about 1 uM to about 40 uM of the one or more cGAS-STING pathway inhibitors. In embodiments, the population of T cells is independently contacted with about 1 uM to about 38 uM of the one or more cGAS-STING pathway inhibitors. In embodiments, the population of T cells is independently contacted with about 1 uM to about 36 uM of the one or more cGAS-STING pathway inhibitors. In embodiments, the population of T cells is independently contacted with about 1 uM to about 32 uM of the one or more cGAS-STING pathway inhibitors. In embodiments, the population of T cells is independently contacted with about 1 uM to about 30 uM of the one or more cGAS-STING pathway inhibitors. In embodiments, the population of T cells is independently contacted with about 1 uM to about 28 uM of the one or more cGAS-STING pathway inhibitors. In embodiments, the population of T cells is independently contacted with about 1 uM to about 26 uM of the one or more cGAS-STING pathway inhibitors. In embodiments, the population of T cells is independently contacted with about 1 uM to about 24 uM of the one or more cGAS-STING pathway inhibitors. In embodiments, the population of T cells is independently contacted with about 1 uM to about 22 uM of the one or more cGAS-STING pathway inhibitors. In embodiments, the population of T cells is independently contacted with about 1 uM to about 20 uM of the one or more cGAS-STING pathway inhibitors. In embodiments, the population of T cells is independently contacted with about 1 uM to about 18 uM of the one or more cGAS-STING pathway inhibitors. In embodiments, the population of T cells is independently contacted with about 1 uM to about 16 uM of the one or more cGAS-STING pathway inhibitors. In embodiments, the population of T cells is independently contacted with about 1 uM to about 14 uM of the one or more cGAS-STING pathway inhibitors. In embodiments, the population of T cells is independently contacted with about 1 uM to about 12 uM of the one or more cGAS-STING pathway inhibitors. In embodiments, the population of T cells is independently contacted with about 1 uM to about 10 uM of the one or more cGAS-STING pathway inhibitors. In embodiments, the population of T cells is independently contacted with about 1 uM to about 8 uM of the one or more cGAS-STING pathway inhibitors. In embodiments, the population of T cells is independently contacted with about 1 uM to about 6 uM of the one or more cGAS-STING pathway inhibitors. In embodiments, the population of T cells is independently contacted with about 1 uM to about 4 uM of the one or more cGAS-STING pathway inhibitors. In embodiments, the population of T cells is independently contacted with about 1 uM to about 2 uM of the one or more cGAS-STING pathway inhibitors. In embodiments, the population of T cells is independently contacted with about 1 uM, 2 uM, 4 uM, 6 uM, 8 uM, 10 uM, 12 uM, 14 uM, 16 uM, 18 uM, 20 uM, 22 uM, 24 uM, 26 uM, 28 uM, 30 uM, 32 uM, 34 uM, 36 uM, 38 uM, 40 uM, 42 uM, 44 uM, 46 uM, 48 uM, or 50 uM of the one or more cGAS-STING pathway inhibitors.

In embodiments, the population of T cells is contacted with the nucleic acid in the presence of the one or more cGAS-STING pathway inhibitors. In embodiments, the population of T cells is contacted sequentially with the nucleic acid and the one or more cGAS-STING pathway inhibitors. In embodiments, the population of T cells is contacted with the one or more cGAS-STING pathway inhibitors prior to the nucleic acid.

In embodiments, the T cell is contacted with one cGAS-STING pathway inhibitor. In embodiments, the T cell is contacted with one cGAS-STING pathway inhibitor as provided herein and no other cGAS-STING pathway inhibitor.

In embodiments, the population of T cells is contacted with about 1 uM to about 10 uM BX795. In embodiments, the population of T cells is contacted with about 0.5 uM to about 10 uM BX795. In embodiments, the population of T cells is contacted with about 2 uM to about 10 uM BX795. In embodiments, the population of T cells is contacted with about 2.5 uM to about 10 uM BX795. In embodiments, the population of T cells is contacted with about 3 uM to about 10 uM BX795. In embodiments, the population of T cells is contacted with about 3.5 uM to about 10 uM BX795. In embodiments, the population of T cells is contacted with about 4 uM to about 10 uM BX795. In embodiments, the population of T cells is contacted with about 4.5 uM to about 10 uM BX795. In embodiments, the population of T cells is contacted with about 5 uM to about 10 uM BX795. In embodiments, the population of T cells is contacted with about 5.5 uM to about 10 uM BX795. In embodiments, the population of T cells is contacted with about 6 uM to about 10 uM BX795. In embodiments, the population of T cells is contacted with about 6.5 uM to about 10 uM BX795. In embodiments, the population of T cells is contacted with about 7 uM to about 10 uM BX795. In embodiments, the population of T cells is contacted with about 7.5 uM to about 10 uM BX795. In embodiments, the population of T cells is contacted with about 8 uM to about 10 uM BX795. In embodiments, the population of T cells is contacted with about 8.5 uM to about 10 uM BX795. In embodiments, the population of T cells is contacted with about 8 uM to about 10 uM BX795. In embodiments, the population of T cells is contacted with about 8.5 uM to about 10 uM BX795. In embodiments, the population of T cells is contacted with about 9 uM to about 10 uM BX795. In embodiments, the population of T cells is contacted with about 9.5 uM to about 10 uM BX795.

In embodiments, the population of T cells is contacted with about 1 uM to about 9.5 uM BX795. In embodiments, the population of T cells is contacted with about 0.5 uM to about 9 uM BX795. In embodiments, the population of T cells is contacted with about 1 uM to about 8.5 uM BX795. In embodiments, the population of T cells is contacted with about 0.5 uM to about 8 uM BX795. In embodiments, the population of T cells is contacted with about 1 uM to about 7.5 uM BX795. In embodiments, the population of T cells is contacted with about 0.5 uM to about 7 uM BX795. In embodiments, the population of T cells is contacted with about 1 uM to about 6.5 uM BX795. In embodiments, the population of T cells is contacted with about 0.5 uM to about 6 uM BX795. In embodiments, the population of T cells is contacted with about 1 uM to about 5.5 uM BX795. In embodiments, the population of T cells is contacted with about 0.5 uM to about 5 uM BX795. In embodiments, the population of T cells is contacted with about 1 uM to about 4.5 uM BX795. In embodiments, the population of T cells is contacted with about 0.5 uM to about 4 uM BX795. In embodiments, the population of T cells is contacted with about 1 uM to about 3.5 uM BX795. In embodiments, the population of T cells is contacted with about 0.5 uM to about 3 uM BX795. In embodiments, the population of T cells is contacted with about 1 uM to about 2.5 uM BX795. In embodiments, the population of T cells is contacted with about 0.5 uM to about 2 uM BX795. In embodiments, the population of T cells is contacted with about 1 uM to about 1.5 uM BX795. In embodiments, the population of T cells is contacted with about 0.5 uM to about 1 uM BX795. In embodiments, the population of T cells is contacted with about 1 uM, 1.5 uM, 2 uM, 2.5 uM, 3 uM, 3.5 uM, 4 uM, 4.5 uM, 5 uM, 5.5 uM, 6 uM, 6.5 uM, 7 uM, 7.5 uM, 8 uM, 8.5 uM, 9 uM, 9.5 uM, or 10 uM BX795.

In embodiments, the population of T cells is contacted with about 0.1 uM to about 8 uM ODNA151. In embodiments, the population of T cells is contacted with about 0.5 uM to about 8 uM ODNA151. In embodiments, the population of T cells is contacted with about 1 uM to about 8 uM ODNA151. In embodiments, the population of T cells is contacted with about 1.5 uM to about 8 uM ODNA151. In embodiments, the population of T cells is contacted with about 2 uM to about 8 uM ODNA151. In embodiments, the population of T cells is contacted with about 2.5 uM to about 8 uM ODNA151. In embodiments, the population of T cells is contacted with about 3 uM to about 8 uM ODNA151. In embodiments, the population of T cells is contacted with about 3.5 uM to about 8 uM ODNA151. In embodiments, the population of T cells is contacted with about 4 uM to about 8 uM ODNA151. In embodiments, the population of T cells is contacted with about 4.5 uM to about 8 uM ODNA151. In embodiments, the population of T cells is contacted with about 5 uM to about 8 uM ODNA151. In embodiments, the population of T cells is contacted with about 5.5 uM to about 8 uM ODNA151. In embodiments, the population of T cells is contacted with about 6 uM to about 8 uM ODNA151. In embodiments, the population of T cells is contacted with about 6.5 uM to about 8 uM ODNA151. In embodiments, the population of T cells is contacted with about 7 uM to about 8 uM ODNA151. In embodiments, the population of T cells is contacted with about 7.5 uM to about 8 uM ODNA151.

In embodiments, the population of T cells is contacted with about 0.1 uM to about 7.5 uM ODNA151. In embodiments, the population of T cells is contacted with about 0.1 uM to about 7 uM ODNA151. In embodiments, the population of T cells is contacted with about 0.1 uM to about 7.5 uM ODNA151. In embodiments, the population of T cells is contacted with about 0.1 uM to about 7 uM ODNA151. In embodiments, the population of T cells is contacted with about 0.1 uM to about 6.5 uM ODNA151. In embodiments, the population of T cells is contacted with about 0.1 uM to about 6 uM ODNA151. In embodiments, the population of T cells is contacted with about 0.1 uM to about 5.5 uM ODNA151. In embodiments, the population of T cells is contacted with about 0.1 uM to about 5 uM ODNA151. In embodiments, the population of T cells is contacted with about 0.1 uM to about 4.5 uM ODNA151. In embodiments, the population of T cells is contacted with about 0.1 uM to about 4 uM ODNA151. In embodiments, the population of T cells is contacted with about 0.1 uM to about 3.5 uM ODNA151. In embodiments, the population of T cells is contacted with about 0.1 uM to about 3 uM ODNA151. In embodiments, the population of T cells is contacted with about 0.1 uM to about 2.5 uM ODNA151. In embodiments, the population of T cells is contacted with about 0.1 uM to about 2 uM ODNA151. In embodiments, the population of T cells is contacted with about 0.1 uM to about 1.5 uM ODNA151. In embodiments, the population of T cells is contacted with about 0.1 uM to about 1 uM ODNA151. In embodiments, the population of T cells is contacted with about 0.1 uM to about 0.5 uM ODNA151. In embodiments, the population of T cells is contacted with about 0.1 uM, 0.5 uM, 1 uM, 1.5 uM, 2 uM, 2.5 uM, 3 uM, 3.5 uM, 4 uM, 4.5 uM, 5 uM, 5.5 uM, 6 uM, 6.5 uM, 7 uM, 7.5 uM, or 8 uM ODNA151.

In embodiments, the population of T cells is contacted with about 5 uM to about 50 uM AML. In embodiments, the population of T cells is contacted with about 15 uM to about 50 uM AML. In embodiments, the population of T cells is contacted with about 20 uM to about 50 uM AML. In embodiments, the population of T cells is contacted with about 25 uM to about 50 uM AML. In embodiments, the population of T cells is contacted with about 30 uM to about 50 uM AML. In embodiments, the population of T cells is contacted with about 35 uM to about 50 uM AML. In embodiments, the population of T cells is contacted with about 40 uM to about 50 uM AML. In embodiments, the population of T cells is contacted with about 45 uM to about 50 uM AML.

In embodiments, the population of T cells is contacted with about 5 uM to about 45 uM AML. In embodiments, the population of T cells is contacted with about 5 uM to about 40 uM AML. In embodiments, the population of T cells is contacted with about 5 uM to about 35 uM AML. In embodiments, the population of T cells is contacted with about 5 uM to about 30 uM AML. In embodiments, the population of T cells is contacted with about 5 uM to about 25 uM AML. In embodiments, the population of T cells is contacted with about 5 uM to about 20 uM AML. In embodiments, the population of T cells is contacted with about 5 uM to about 15 uM AML. In embodiments, the population of T cells is contacted with about 5 uM to about 10 uM AML. In embodiments, the population of T cells is contacted with about 5 uM, 10 uM, 15 uM, 20 uM, 25 uM, 30 uM, 35 uM, 40 uM, 45 uM, or 50 uM AML.

In embodiments, the population of T cells is contacted with about 1 uM to about 25 uM. MRT. In embodiments, the population of T cells is contacted with about 2 uM to about 25 uM MRT. In embodiments, the population of T cells is contacted with about 3 uM to about 25 uM MRT. In embodiments, the population of T cells is contacted with about 4 uM to about 25 uM MRT. In embodiments, the population of T cells is contacted with about 5 uM to about 25 uM MRT. In embodiments, the population of T cells is contacted with about 6 uM to about 25 uM MRT. In embodiments, the population of T cells is contacted with about 7 uM to about 25 uM MRT. In embodiments, the population of T cells is contacted with about 8 uM to about 25 uM MRT. In embodiments, the population of T cells is contacted with about 9 uM to about 25 uM MRT. In embodiments, the population of T cells is contacted with about 10 uM to about 25 uM MRT. In embodiments, the population of T cells is contacted with about 11 uM to about 25 uM MRT. In embodiments, the population of T cells is contacted with about 12 uM to about 25 uM MRT. In embodiments, the population of T cells is contacted with about 13 uM to about 25 uM MRT. In embodiments, the population of T cells is contacted with about 14 uM to about 25 uM MRT. In embodiments, the population of T cells is contacted with about 15 uM to about 25 uM MRT. In embodiments, the population of T cells is contacted with about 16 uM to about 25 uM MRT. In embodiments, the population of T cells is contacted with about 17 uM to about 25 uM MRT. In embodiments, the population of T cells is contacted with about 18 uM to about 25 uM MRT. In embodiments, the population of T cells is contacted with about 19 uM to about 25 uM MRT. In embodiments, the population of T cells is contacted with about 20 uM to about 25 uM MRT. In embodiments, the population of T cells is contacted with about 21 uM to about 25 uM MRT. In embodiments, the population of T cells is contacted with about 22 uM to about 25 uM MRT. In embodiments, the population of T cells is contacted with about 23 uM to about 25 uM MRT. In embodiments, the population of T cells is contacted with about 24 uM to about 25 uM MRT.

In embodiments, the population of T cells is contacted with about 1 uM to about 24 uM MRT. In embodiments, the population of T cells is contacted with about 1 uM to about 23 uM MRT. In embodiments, the population of T cells is contacted with about 1 uM to about 22 uM MRT. In embodiments, the population of T cells is contacted with about 1 uM to about 21 uM MRT. In embodiments, the population of T cells is contacted with about 1 uM to about 20 uM MRT. In embodiments, the population of T cells is contacted with about 1 uM to about 19 uM MRT. In embodiments, the population of T cells is contacted with about 1 uM to about 18 uM MRT. In embodiments, the population of T cells is contacted with about 1 uM to about 17 uM MRT. In embodiments, the population of T cells is contacted with about 1 uM to about 16 uM MRT. In embodiments, the population of T cells is contacted with about 1 uM to about 15 uM MRT. In embodiments, the population of T cells is contacted with about 1 uM to about 14 uM MRT. In embodiments, the population of T cells is contacted with about 1 uM to about 13 uM MRT. In embodiments, the population of T cells is contacted with about 1 uM to about 12 uM MRT. In embodiments, the population of T cells is contacted with about 1 uM to about 11 uM MRT. In embodiments, the population of T cells is contacted with about 1 uM to about 10 uM MRT. In embodiments, the population of T cells is contacted with about 1 uM to about 9 uM MRT. In embodiments, the population of T cells is contacted with about 1 uM to about 8 uM MRT. In embodiments, the population of T cells is contacted with about 1 uM to about 7 uM MRT. In embodiments, the population of T cells is contacted with about 1 uM to about 6 uM MRT. In embodiments, the population of T cells is contacted with about 1 uM to about 5 uM MRT. In embodiments, the population of T cells is contacted with about 1 uM to about 4 uM MRT. In embodiments, the population of T cells is contacted with about 1 uM to about 3 uM MRT. In embodiments, the population of T cells is contacted with about 1 uM to about 2 uM MRT. In embodiments, the population of T cells is contacted with about 1 uM, 2 uM, 3 uM, 4 uM, 5 uM, 6 uM, 7 uM, 8 uM, 9 uM, 10 uM, 11 uM, 12 uM, 13 uM, 14 uM, 15 uM, 16 uM, 17 uM, 18 uM, 19 uM, 20 uM, 21 uM, 22 uM, 23 uM, 24 uM, or 25 uM MRT.

In embodiments, the population of expanded engineered T cells is increased from at least about 0.5 fold to at least about 5 fold relative to a population of engineered T cells, wherein the population of T cells of step i) are not contacted with one or more cGAS-STING pathway inhibitors. In embodiments, the population of expanded engineered T cells is increased from at least about 1 fold to at least about 5 fold relative to a population of engineered T cells, wherein the population of T cells of step i) are not contacted with one or more cGAS-STING pathway inhibitors. In embodiments, the population of expanded engineered T cells is increased from at least about 1.5 fold to at least about 5 fold relative to a population of engineered T cells, wherein the population of T cells of step i) are not contacted with one or more cGAS-STING pathway inhibitors. In embodiments, the population of expanded engineered T cells is increased from at least about 2 fold to at least about 5 fold relative to a population of engineered T cells, wherein the population of T cells of step i) are not contacted with one or more cGAS-STING pathway inhibitors. In embodiments, the population of expanded engineered T cells is increased from at least about 2.5 fold to at least about 5 fold relative to a population of engineered T cells, wherein the population of T cells of step i) are not contacted with one or more cGAS-STING pathway inhibitors. In embodiments, the population of expanded engineered T cells is increased from at least about 3 fold to at least about 5 fold relative to a population of engineered T cells, wherein the population of T cells of step i) are not contacted with one or more cGAS-STING pathway inhibitors. In embodiments, the population of expanded engineered T cells is increased from at least about 3.5 fold to at least about 5 fold relative to a population of engineered T cells, wherein the population of T cells of step i) are not contacted with one or more cGAS-STING pathway inhibitors. In embodiments, the population of expanded engineered T cells is increased from at least about 4 fold to at least about 5 fold relative to a population of engineered T cells, wherein the population of T cells of step i) are not contacted with one or more cGAS-STING pathway inhibitors. In embodiments, the population of expanded engineered T cells is increased from at least about 4.5 fold to at least about 5 fold relative to a population of engineered T cells, wherein the population of T cells of step i) are not contacted with one or more cGAS-STING pathway inhibitors.

In embodiments, the population of expanded engineered T cells is increased from at least about 0.5 fold to at least about 4.5 fold relative to a population of engineered T cells, wherein the population of T cells of step i) are not contacted with one or more cGAS-STING pathway inhibitors. In embodiments, the population of expanded engineered T cells is increased from at least about 0.5 fold to at least about 4 fold relative to a population of engineered T cells, wherein the population of T cells of step i) are not contacted with one or more cGAS-STING pathway inhibitors. In embodiments, the population of expanded engineered T cells is increased from at least about 0.5 fold to at least about 3.5 fold relative to a population of engineered T cells, wherein the population of T cells of step i) are not contacted with one or more cGAS-STING pathway inhibitors. In embodiments, the population of expanded engineered T cells is increased from at least about 0.5 fold to at least about 3 fold relative to a population of engineered T cells, wherein the population of T cells of step i) are not contacted with one or more cGAS-STING pathway inhibitors. In embodiments, the population of expanded engineered T cells is increased from at least about 0.5 fold to at least about 2.5 fold relative to a population of engineered T cells, wherein the population of T cells of step i) are not contacted with one or more cGAS-STING pathway inhibitors. In embodiments, the population of expanded engineered T cells is increased from at least about 0.5 fold to at least about 2 fold relative to a population of engineered T cells, wherein the population of T cells of step i) are not contacted with one or more cGAS-STING pathway inhibitors. In embodiments, the population of expanded engineered T cells is increased from at least about 0.5 fold to at least about 1.5 fold relative to a population of engineered T cells, wherein the population of T cells of step i) are not contacted with one or more cGAS-STING pathway inhibitors. In embodiments, the population of expanded engineered T cells is increased from at least about 0.5 fold to at least about 1 fold relative to a population of engineered T cells, wherein the population of T cells of step i) are not contacted with one or more cGAS-STING pathway inhibitors. In embodiments, the population of expanded engineered T cells is increased from at least about 0.5 fold, 1 fold, 2 fold, 2.5 fold, 3 fold, 3.5 fold, 4 fold, 4.5 fold or 5 fold relative to a population of engineered T cells, wherein the population of T cells of step i) are not contacted with one or more cGAS-STING pathway inhibitors.

For the methods provided herein, in embodiments, the population of expanded engineered T cells is increased from about 2 fold to about 3 fold relative to a population of engineered T cells, wherein the population of T cells of step i) are not contacted with one or more cGAS-STING pathway inhibitors.

In embodiments, the population of engineered T cells are expanded from at least about 0.5 fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about fold to at least about 100 fold relative to a population of engineered T cells, wherein the population of T cells of step i) are not contacted with one or more cGAS-STING pathway inhibitors. In embodiments, the population of engineered T cells are expanded from at least about 55 fold to at least about 100 fold relative to a population of engineered T cells, wherein the population of T cells of step i) are not contacted with one or more cGAS-STING pathway inhibitors. In embodiments, the population of engineered T cells are expanded from at least about 60 fold to at least about 100 fold relative to a population of engineered T cells, wherein the population of T cells of step i) are not contacted with one or more cGAS-STING pathway inhibitors. In embodiments, the population of engineered T cells are expanded from at least about 65 fold to at least about 100 fold relative to a population of engineered T cells, wherein the population of T cells of step i) are not contacted with one or more cGAS-STING pathway inhibitors. In embodiments, the population of engineered T cells are expanded from at least about 70 fold to at least about 100 fold relative to a population of engineered T cells, wherein the population of T cells of step i) are not contacted with one or more cGAS-STING pathway inhibitors. In embodiments, the population of engineered T cells are expanded from at least about 75 fold to at least about 100 fold relative to a population of engineered T cells, wherein the population of T cells of step i) are not contacted with one or more cGAS-STING pathway inhibitors. In embodiments, the population of engineered T cells are expanded from at least about 80 fold to at least about 100 fold relative to a population of engineered T cells, wherein the population of T cells of step i) are not contacted with one or more cGAS-STING pathway inhibitors. In embodiments, the population of engineered T cells are expanded from at least about 85 fold to at least about 100 fold relative to a population of engineered T cells, wherein the population of T cells of step i) are not contacted with one or more cGAS-STING pathway inhibitors. In embodiments, the population of engineered T cells are expanded from at least about 90 fold to at least about 100 fold relative to a population of engineered T cells, wherein the population of T cells of step i) are not contacted with one or more cGAS-STING pathway inhibitors. In embodiments, the population of engineered T cells are expanded from at least about 95 fold to at least about 100 fold relative to a population of engineered T cells, wherein the population of T cells of step i) are not contacted with one or more cGAS-STING pathway inhibitors.

In embodiments, the population of engineered T cells are expanded from at least about 0.5 fold to at least about 95 fold relative to a population of engineered T cells, wherein the population of T cells of step i) are not contacted with one or more cGAS-STING pathway inhibitors. In embodiments, the population of engineered T cells are expanded from at least about 0.5 fold to at least about 90 fold relative to a population of engineered T cells, wherein the population of T cells of step i) are not contacted with one or more cGAS-STING pathway inhibitors. In embodiments, the population of engineered T cells are expanded from at least about 0.5 fold to at least about 85 fold relative to a population of engineered T cells, wherein the population of T cells of step i) are not contacted with one or more cGAS-STING pathway inhibitors. In embodiments, the population of engineered T cells are expanded from at least about 0.5 fold to at least about 80 fold relative to a population of engineered T cells, wherein the population of T cells of step i) are not contacted with one or more cGAS-STING pathway inhibitors. In embodiments, the population of engineered T cells are expanded from at least about 0.5 fold to at least about 75 fold relative to a population of engineered T cells, wherein the population of T cells of step i) are not contacted with one or more cGAS-STING pathway inhibitors. In embodiments, the population of engineered T cells are expanded from at least about 0.5 fold to at least about 70 fold relative to a population of engineered T cells, wherein the population of T cells of step i) are not contacted with one or more cGAS-STING pathway inhibitors. In embodiments, the population of engineered T cells are expanded from at least about 0.5 fold to at least about 65 fold relative to a population of engineered T cells, wherein the population of T cells of step i) are not contacted with one or more cGAS-STING pathway inhibitors. In embodiments, the population of engineered T cells are expanded from at least about 0.5 fold to at least about 60 fold relative to a population of engineered T cells, wherein the population of T cells of step i) are not contacted with one or more cGAS-STING pathway inhibitors. In embodiments, the population of engineered T cells are expanded from at least about 0.5 fold to at least about 55 fold relative to a population of engineered T cells, wherein the population of T cells of step i) are not contacted with one or more cGAS-STING pathway inhibitors. In embodiments, the population of engineered T cells are expanded from at least about 0.5 fold to at least about 50 fold relative to a population of engineered T cells, wherein the population of T cells of step i) are not contacted with one or more cGAS-STING pathway inhibitors. In embodiments, the population of engineered T cells are expanded from at least about 0.5 fold to at least about 45 fold relative to a population of engineered T cells, wherein the population of T cells of step i) are not contacted with one or more cGAS-STING pathway inhibitors. In embodiments, the population of engineered T cells are expanded from at least about 0.5 fold to at least about 40 fold relative to a population of engineered T cells, wherein the population of T cells of step i) are not contacted with one or more cGAS-STING pathway inhibitors. In embodiments, the population of engineered T cells are expanded from at least about 0.5 fold to at least about 35 fold relative to a population of engineered T cells, wherein the population of T cells of step i) are not contacted with one or more cGAS-STING pathway inhibitors. In embodiments, the population of engineered T cells are expanded from at least about 0.5 fold to at least about 30 fold relative to a population of engineered T cells, wherein the population of T cells of step i) are not contacted with one or more cGAS-STING pathway inhibitors. In embodiments, the population of engineered T cells are expanded from at least about 0.5 fold to at least about 25 fold relative to a population of engineered T cells, wherein the population of T cells of step i) are not contacted with one or more cGAS-STING pathway inhibitors. In embodiments, the population of engineered T cells are expanded from at least about 0.5 fold to at least about 20 fold relative to a population of engineered T cells, wherein the population of T cells of step i) are not contacted with one or more cGAS-STING pathway inhibitors. In embodiments, the population of engineered T cells are expanded from at least about 0.5 fold to at least about 15 fold relative to a population of engineered T cells, wherein the population of T cells of step i) are not contacted with one or more cGAS-STING pathway inhibitors. In embodiments, the population of engineered T cells are expanded from at least about 0.5 fold to at least about 10 fold relative to a population of engineered T cells, wherein the population of T cells of step i) are not contacted with one or more cGAS-STING pathway inhibitors. In embodiments, the population of engineered T cells are expanded from at least about 0.5 fold to at least about 5 fold relative to a population of engineered T cells, wherein the population of T cells of step i) are not contacted with one or more cGAS-STING pathway inhibitors. In embodiments, the population of engineered T cells are expanded at least about 0.5 fold, 5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold relative to a population of engineered T cells, wherein the population of T cells of step i) are not contacted with one or more cGAS-STING pathway inhibitors.

### Engineered T Cell Compositions

Provided herein, inter alia, are compositions including an engineered T cell made by a method provided herein including embodiments thereof. In an aspect is provided an engineered T cell, made by a method provided herein including embodiments thereof.

Provided herein, inter alia, are compositions including a population of engineered T cells made by a method provided herein including embodiments thereof. The population of engineered T cells may have increased viability and/or expansion compared to a population of engineered T cells made by a method wherein a population T cells are not contacted with a cGAS-STING pathway inhibitor prior to generation of the population of engineered T cells. Thus, in an aspect is provided a population of engineered T cells made by contacting a population of T cells with a nucleic acid and one or more cyclic GMP-AMP synthase (cGAS)-stimulator of interferon gene (STING) pathway inhibitors.

In embodiments, the nucleic acid includes a donor DNA. In embodiments, the nucleic acid is a plasmid, a nanoplasmid, a minicircle, or a viral vector including a donor DNA. In embodiments, the donor DNA encodes an exogenous T cell receptor (TCR)-alpha or a fragment thereof, an exogenous TCR-beta or a fragment thereof, or a combination thereof. In embodiments, the nucleic acid is associated with a delivery vehicle. In embodiments, the delivery vehicle is a lipid particle or a nanoparticle.

In embodiments, the population of T cells comprises primary T cells.

For the population of engineered T cells provided herein, in embodiments, the population of T cells is further contacted with a gene editing reagent. In embodiments, the donor DNA is inserted into an endogenous TCR locus. In embodiments, the endogenous TCR locus is an endogenous TCR-alpha locus, an endogenous TCR-beta locus, or a combination thereof.

In embodiments, contacting the T cell with the gene editing reagent includes transfecting the T cell with the gene editing reagent. In embodiments, contacting the T cell with the nucleic acid includes transfecting the T cell with the nucleic acid.

In embodiments, the one or more cGAS-STING pathway inhibitors includes a cGAS inhibitor. In embodiments, the one or more cGAS-STING pathway inhibitors includes a STING inhibitor. In embodiments, the one or more cGAS-STING pathway inhibitors includes a TANK-binding kinase 1 (TBK1) inhibitor.

In embodiments, the one or more cGAS-STING pathway inhibitors is Amlexanox (Aml), MRT67307 (MRT), BX795, H151, ODN-A151 (ODN151), Ru.521, G140, or combinations thereof. In embodiments, the one or more cGAS-STING pathway inhibitors is selected from: Aml, BX795, ODN151, and MRT. In embodiments, the one or more cGAS-STING pathway inhibitors is ODN151. In embodiments, the one or more cGAS-STING pathway inhibitors is BX795.

In embodiments, the population of T cells and the nucleic acid are contacted in the presence of one or more cGAS-STING pathway inhibitors. In embodiments, the population of T cells is contacted sequentially with the nucleic acid and the one or more cGAS-STING pathway inhibitors. In embodiments, the population of T cells is contacted with the one or more cGAS-STING pathway inhibitors prior to the nucleic acid.

In embodiments, the population of T cells is contacted with the one or more cGAS-STING pathway inhibitors for up to about 10 hours, e.g., about 2 hours to about 10 hours. In embodiments, the population of T cells is contacted with the one or more cGAS-STING pathway inhibitors for about 3 hours to about 10 hours. In embodiments, the population of T cells is contacted with the one or more cGAS-STING pathway inhibitors for about 4 hours to about 10 hours. In embodiments, the population of T cells is contacted with the one or more cGAS-STING pathway inhibitors for about 5 hours to about 10 hours. In embodiments, the population of T cells is contacted with the one or more cGAS-STING pathway inhibitors for about 6 hours to about 10 hours. In embodiments, the population of T cells is contacted with the one or more cGAS-STING pathway inhibitors for about 7 hours to about 10 hours. In embodiments, the population of T cells is contacted with the one or more cGAS-STING pathway inhibitors for about 8 hours to about 10 hours. In embodiments, the population of T cells is contacted with the one or more cGAS-STING pathway inhibitors for about 9 hours to about 10 hours.

In embodiments, the population of T cells is contacted with the one or more cGAS-STING pathway inhibitors for up to about 9 hours, e.g., about 2 hours to about 9 hours. In embodiments, the population of T cells is contacted with the one or more cGAS-STING pathway inhibitors for about 2 hours to about 8 hours. In embodiments, the population of T cells is contacted with the one or more cGAS-STING pathway inhibitors for about 2 hours to about 7 hours. In embodiments, the population of T cells is contacted with the one or more cGAS-STING pathway inhibitors for about 2 hours to about 6 hours. In embodiments, the population of T cells is contacted with the one or more cGAS-STING pathway inhibitors for about 2 hours to about 5 hours. In embodiments, the population of T cells is contacted with the one or more cGAS-STING pathway inhibitors for about 2 hours to about 4 hours. In embodiments, the population of T cells is contacted with the one or more cGAS-STING pathway inhibitors for about 2 hours to about 3 hours. In embodiments, the population of T cells is contacted with the one or more cGAS-STING pathway inhibitors for about 2 hours, 3 hours, 4 hours, 5 hours, 6 hours, 7 hours, 8 hours, 9 hours, or 10 hours. In embodiments, the T cell is contacted with the one or more cGAS-STING pathway inhibitors for about 6 hours. In embodiments, the T cell is contacted with the one or more cGAS-STING pathway inhibitors for 6 hours.

In embodiments, the population of T cells is contacted with one cGAS-STING pathway inhibitor. In embodiments, the population of T cells is contacted with one cGAS-STING pathway inhibitor and no other cGAS-STING pathway inhibitors.

### T Cell Compositions

Provided herein are compositions including a population of T cells and one or more cGAS-STING inhibitors, wherein the compositions are useful for generating a population of engineered T cells. Applicant has demonstrated that the one or more cGAS-STING inhibitors increase gene editing efficiency in the T cells. Application has further demonstrated that the compositions provided herein including embodiments thereof result in a population of engineered T cells with increased cell viability and expansion compared to compositions that do not include one or more cGAS-STING inhibitors. Thus, in an aspect is provided a composition including a population of T cells, a nucleic acid, and one or more cyclic GMP-AMP synthase (cGAS)-stimulator of interferon gene (STING) pathway inhibitors.

In embodiments, the nucleic acid comprises a donor DNA. In embodiments, the nucleic acid is a plasmid, a nanoplasmid, a minicircle, or a viral vector comprising a donor DNA. In embodiments, the donor DNA encodes an exogenous T cell receptor (TCR)-alpha or a fragment thereof, an exogenous TCR-beta or a fragment thereof, or a combination thereof. In embodiments, the nucleic acid is associated with a delivery vehicle. In embodiments, the delivery vehicle is a lipid particle or a nanoparticle.

In embodiments, the population of T cells includes primary T cells.

In embodiments, the composition further includes a gene editing reagent.

In embodiments, the one or more cGAS-STING pathway inhibitors includes a cGAS inhibitor. In embodiments, the one or more cGAS-STING pathway inhibitors includes a STING inhibitor. In embodiments, the one or more cGAS-STING pathway inhibitors includes a TANK-binding kinase 1 (TBK1) inhibitor.

In embodiments, the one or more cGAS-STING pathway inhibitors is Amlexanox (Aml), MRT67307 (MRT), BX795, H151, ODN-A151 (ODN151), Ru.521, G140, or combinations thereof. In embodiments, the one or more cGAS-STING pathway inhibitors includes Aml. In embodiments, the one or more cGAS-STING pathway inhibitors includes MRT. In embodiments, the one or more cGAS-STING pathway inhibitors includes BX795. In embodiments, the one or more cGAS-STING pathway inhibitors includes H151. In embodiments, the one or more cGAS-STING pathway inhibitors includes ODN151. In embodiments, the one or more cGAS-STING pathway inhibitors includes Ru.521. In embodiments, the one or more cGAS-STING pathway inhibitors includes G140.

In embodiments, the cGAS-STING pathway inhibitor is Aml. In embodiments, the cGAS-STING pathway inhibitor is MRT. In embodiments, the cGAS-STING pathway inhibitor is BX795. In embodiments, the cGAS-STING pathway inhibitor is H151. In embodiments, the cGAS-STING pathway inhibitor is ODN151. In embodiments, the cGAS-STING pathway inhibitor is Ru.521. In embodiments, the cGAS-STING pathway inhibitor is G140. In embodiments, the cGAS-STING pathway inhibitor is Aml and no other cGAS-STING pathway inhibitor. In embodiments, the cGAS-STING pathway inhibitor is MRT and no other cGAS-STING pathway inhibitor. In embodiments, the cGAS-STING pathway inhibitor is BX795 and no other cGAS-STING pathway inhibitor. In embodiments, the cGAS-STING pathway inhibitor is H151 and no other cGAS-STING pathway inhibitor. In embodiments, the cGAS-STING pathway inhibitor is ODN151 and no other cGAS-STING pathway inhibitor. In embodiments, the cGAS-STING pathway inhibitor is Ru.521 and no other cGAS-STING pathway inhibitor. In embodiments, the cGAS-STING pathway inhibitor is G140 and no other cGAS-STING pathway inhibitor.

In embodiments, the one or more cGAS-STING pathway inhibitors is selected from: Aml, BX795, ODN151, and MRT. In embodiments, the one or more cGAS-STING pathway inhibitors is ODN151. In embodiments, the one or more cGAS-STING pathway inhibitors is BX795.

### Pharmaceutical Compositions

The compositions provided herein, including T cell compositions and engineered T cell compositions, are contemplated to be effective for treating diseases (e.g. cancer). For example, the engineered T cells provided herein may include exogenous T cell receptors specific for cancer cell antigens. Thus, in an aspect is provided a pharmaceutical composition including the engineered T cell provided herein including embodiments thereof. In embodiments, the pharmaceutical composition further includes a pharmaceutically acceptable excipient (e.g., saline).

### Methods of Treatment

The engineered T cells provided herein including embodiments thereof are contemplated to be specific for disease-specific antigens (e.g. cancer cell antigens), thereby allowing effective targeting of cancer cells. Engineered T cells may include, for example, one or more exogenous T cell receptors engineered to be specific for an individual's cancer cells, allowing personalized and specific targeting of the cancer cells. Thus, in an aspect is provided a method of treating a disease in a subject in need thereof, including administering a therapeutically effective amount of the engineered T cell provided herein including embodiments thereof or the pharmaceutical composition provided herein including embodiments thereof. In embodiments, the method includes administering a therapeutically effective amount of the engineered T cell provided herein including embodiments thereof. In embodiments, the method includes administering a therapeutically effective amount of the pharmaceutical composition provided herein including embodiments thereof.

For the methods provided herein, in embodiments, the engineered T cell may be generated from the subject. For example, a T cell may be extracted from the subject, contacted with a nucleic acid (e.g. donor nucleic acid) and one or more cGAS-STING inhibitors ex vivo thereby generating an engineered T cell, and administered back to the subject. Thus, in embodiments, the engineered T cell is an autologous T cell. In embodiments, the engineered T cell may generated from T cells that are not taken from the subject. For example, the engineered T cell may be generated from a healthy subject (e.g. a subject who does not have cancer). Thus, in embodiments, the engineered T cell is an allogeneic T cell.

For the methods provided herein, in embodiments, the disease is cancer. In embodiments, the cancer is melanoma, lymphoma or leukemia. In embodiments, the cancer is melanoma. In embodiments, the cancer is lymphoma. In embodiments, the cancer is leukemia.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Introduction to Exemplary Studies

Over the past decade, better understanding of tumor immunotherapy combined with the development of human cell engineering techniques has made adoptive cell therapy (ACT) a viable therapeutic option. ACT, a highly personalized cancer therapeutic approach, relies on engineering cancer targeting lymphocytes ex vivo and then administration of these cancer cell killing T cells back to the patients in vivo. Unlike other forms of cancer immunotherapy that depends on developing anti-tumor lymphocytes in the patient's body, which under most circumstances lacks the specificity and efficiency, ACT offers multiple advantages such as high potency, specificity, high number of lymphocytes, and long-lasting circulation time. Nowadays, three forms of ACT are being developed and used in clinical applications: tumor-infiltrating lymphocytes (TILs), chimeric antigen receptor T cells (CAR-T), and T cell receptor engineered T cells (TCR-T). TILs and CAR-Ts have exhibited the potential and achieved encouraging clinical results in patients with melanoma, lymphoma and leukemia, improving both patient's life quality and lifespan. However, serious side effects such as severe cytokine release syndrome (CRS), cardiovascular toxicity, and unexpected neurologic complications are yet to be addressed. Additionally, TILs and CAR-Ts approaches have shown limited success in solid tumor treatment. To overcome these challenges, TCR engineered T cells have been utilized in the past two decades and numerous clinical and preclinical studies have implicated their impact on mediating tumor lysis and eradication in a variety of tumor types. For example, in early 2022 Immunocore Inc.'s Kimmtrak, the first TCR engineered T cell therapeutic approach for melanoma treatment, was approved as a treatment option by FDA.

T cell receptor (TCR) consists of an alpha (α)- and Beta (β) chains associated with CD3 complex on the T cell surface. TCRs noncovalently bind to the peptide-histocompatibility complex class I (MHC-I) molecules on the surface of tumor cells in a highly specific manner, owing to their ability to distinguish between MHC-I molecules that are loaded with none-self or mutation bearing self-peptides versus MHC-I molecules loaded with self-peptides. However, in certain cases the immune surveillance evasion of tumor cells, reduced surface expression of MHC-I molecules, or low levels of mutant self-peptide presentation on the cell surface can curb T cell mediated tumor cell killing. TCR engineering approach aims to solve this problem by knocking-in tumor specific antigen associated TCR while knocking-out the endogenous TCR simultaneously. Nowadays, by utilizing whole genome or exome sequencing techniques, somatic point mutations in tumor tissues are screened and unique peptide antigens, termed neoantigens, with the potential for MHC-I presentation, are identified. Proprietary algorithms are then used to engineer TCRs capable of recognizing MHC-I/neoantigen complexes with the goal to improve the specificity of T cell-mediated tumor cell killing. By further optimizing TCR subtype and intrinsic features, TCR engineered T cells can provide unique therapeutic opportunities that can benefit many patients who struggle with diseases such as synovial cell sarcoma, melanoma, and myeloma.

Many approaches have been developed to improve T cell engineering efficiency. For example, viral vectors have been successfully used for gene-editing purposes but the risk of integrating viral sequences into the human cell genome and the complications that such approaches can pose, cannot be ignored. Use of adeno-associated viruses as the advanced version of viral editing technique has seemingly improved safety concerns, however, it has limitations with regards to gene size and site specific delivery of the target transgenes. Since CAR-T engineering does not have strict requirements with regards to genomic insertion site, viral-based gene editing approaches have been widely used for CAR-T approach. However, more accurate genome editing technology that allows site-specific insertion of transgene(s) is highly desired and not yet fully discovered, and technological needs for site-specific insertion of transgene(s) of interest has yet to be implemented.

Non-viral gene editing, on the other hand, can allow for convenient, safe, and efficient reprogramming of cultured and primary cells. Compared to conventional recombinant viral vectors mentioned above, using electroporation to deliver genome editing reagent such as CRISPR-associated protein 9 (Cas9), guide RNA (gRNA) and DNA templates into the T cells, for instance, can save time, reduce cost, lower safety risks, and enable highly specific genome editing/insertion with ability to integrating much larger size of DNA sequences. Through homology-directed repair (HDR) mechanism, DNA templates can be integrated into the precise site(s), nicked by CRISPR-Cas ribonucleoprotein (RNP, consisting of cas9 protein and gRNA), within the T cell genome. While this approach has been utilized by Roth et al. and others to target human T cells, NK cells and induced pluripotent stem cells (iPSCs), its use in clinical settings has not been widespread as levels of editing efficiencies and off-target activities remain to be determined. Additionally, the functional consequences of electroporation process on cultured T-cells have not been fully examined. Previous studies show that up to 80% of electroporated cells are negatively impacted after electroporation, displaying viability loss, growth reduction, and changes in gene expression. While some correlations between cellular toxicity and DNA format (ssDNA, dsDNA, cDNA), length, and delivery method has been implicated, the underlying cause(s) of post-electroporation cellular toxicity observed in T cell cultures has not been fully characterized. Therefore, there is an urgent need to mitigate cellular toxicity of electroporation during the TCR engineering process in order to enhance gene editing efficiency and cell growth condition.

In this study, we describe an effective, safe, and convenient approach to drastically improve electroporation mediated T cell engineering efficiency, expansion, and hence the total edited cell (TEC) number levels during TCR engineering. Using WT1 peptide associated TCR as a DNA template, we generated a clinically relevant model system where the impact of various components of electroporation-based DNA delivery methods were tested. We investigated the underlying molecular mechanisms of cellular toxicity during the T cell engineering process and identified double stranded DNA as the most potent inducer of cellular DNA genotoxicity in cultured T cells. We subsequently screened several small molecule inhibitors of DNA genotoxicity pathways and analyzed their impact on T cell growth, viability, and TEC numbers to improve TCR engineering efficiency. Additionally, the identified top DNA genotoxicity inhibitors had no negative impact on final drug product (FDP)'s phenotype and function, such as tumor cell killing ability. The aforementioned DNA genotoxicity mitigation approach has shown to be effective in different healthy donors' lymphocyte samples, implying a potential for widespread application(s) in clinical studies.

### Example 2: Inhibition of Genomic Stress Drastically Improves T Cell Engineering Efficiency

### Results

DNA Plasmid Activates cGAS-STING Pathway, which Negatively Affects T Cell Viability and Expansion Post Electroporation

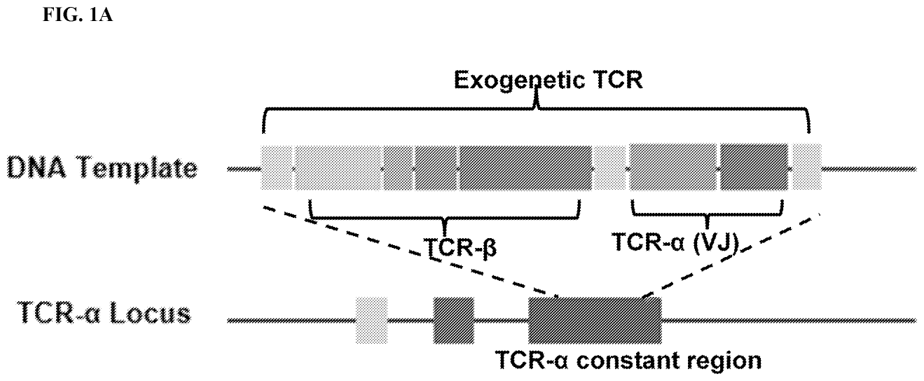

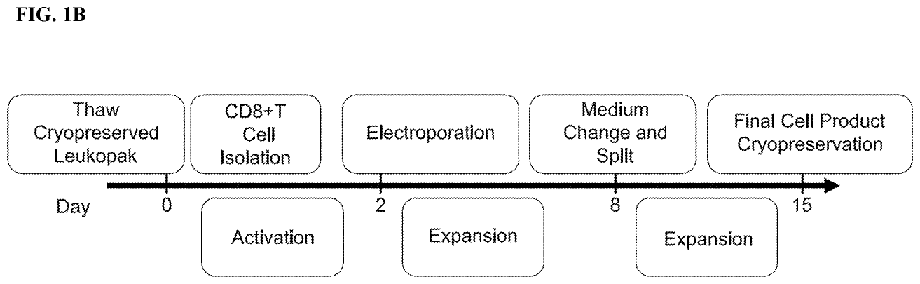

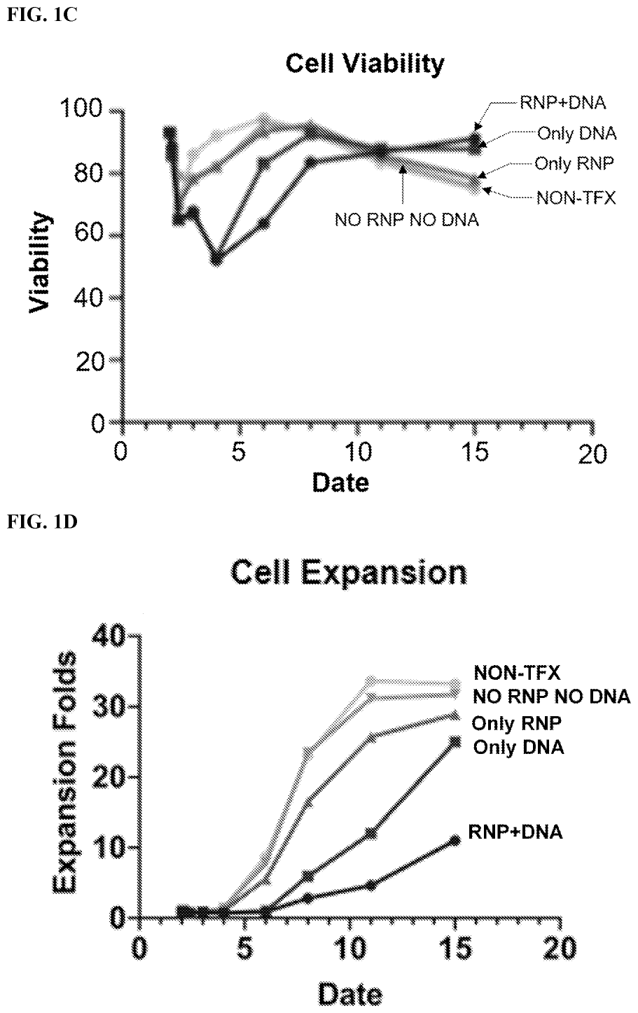

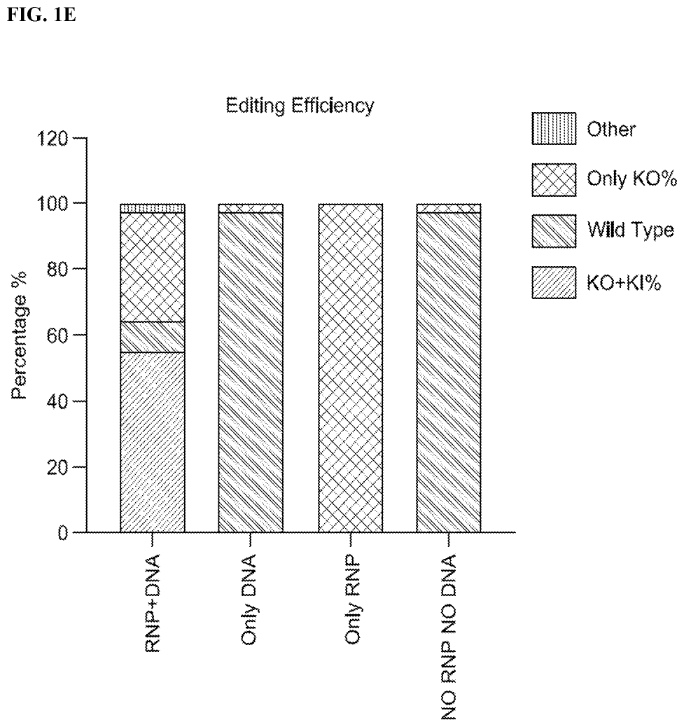

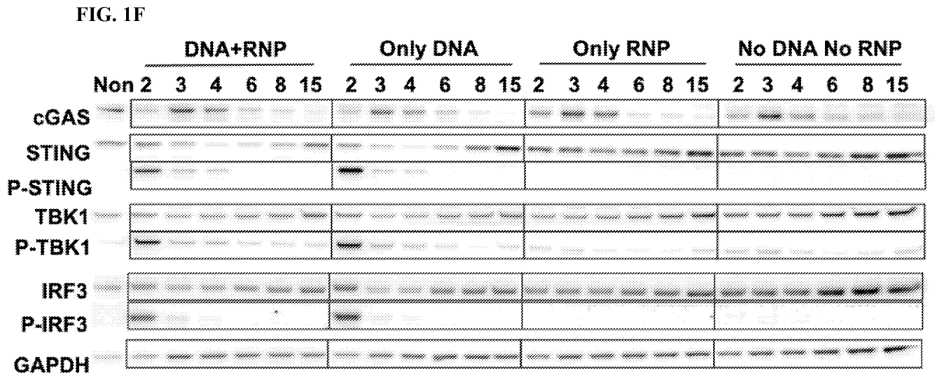

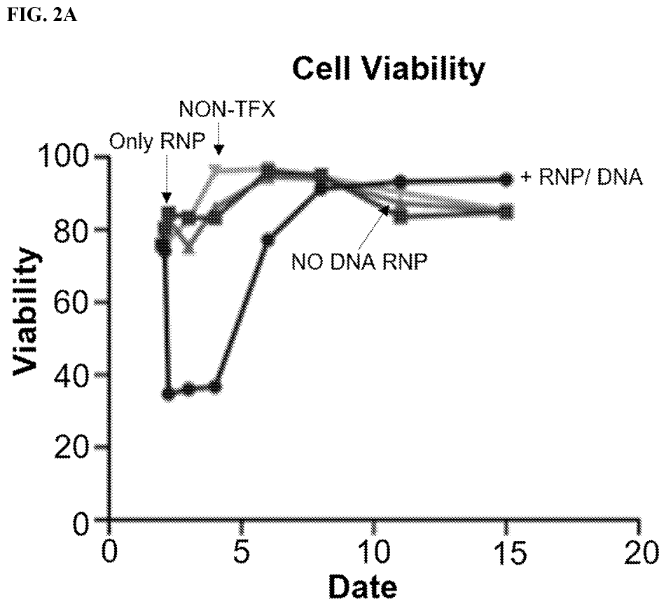

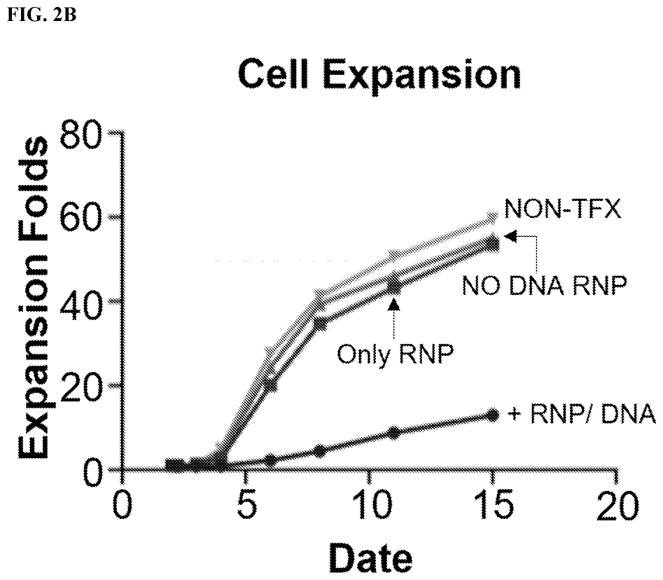

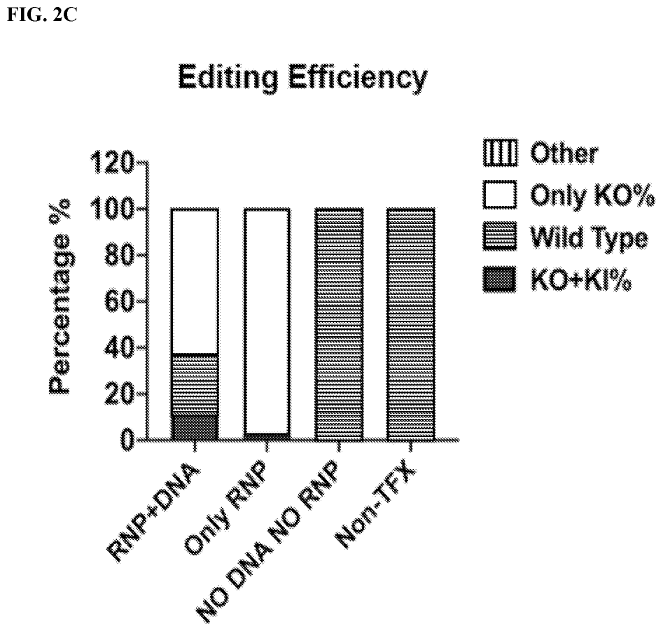

To achieve high gene editing efficiency with low toxicity in a TCR engineering process, we co-electroporated human primary CD8+ T cells with CRISPR-Cas9 ribonucleoprotein (RNP) and nanoplasmid DNA templates, as circular DNA is reportedly less toxic than linear DNA [2,3]. A 1572 bp WT1-peptide-specific TCR template was targeted to TRAC exon 1 (FIG. 1A) through homology-directed repair. The overview of an optimized 15-day process including T cell activation, electroporation, expansion and harvest is shown in FIG. 1B. To address the underlying cause of viability drop and low growth rates after electroporation, we electroporated T cells with different components of CRISPR/Cas9 gene targeting reagents (RNP, DNA template, etc.). The EH115 pulse code was used with the 100 ul cuvette in the Lonza Nucleofector instrument according to the manufacturer's protocol. Viability, T cell growth rate, and knock-in (KI)/knock out (KO) efficiency were measured during this process. Our data showed that the presence of plasmid DNA in the electroporation process (only DNA and DNA+RNP) correlated with a large viability drop and growth reduction in T cells (FIG. 1C, 1D, FIGS. 2A-2C). No drastic impact on viability and growth was observed in the RNP only or control (no DNA, no RNP) groups as they were comparable to the non-transfected (Non-TFX) group. Compared to the DNA only group, RNP+DNA group exhibited a more delayed recovery and reduced growth/expansion rates. These findings suggest that neither the electroporation process nor the RNP were the major reasons for viability and growth reduction in the T cell cultures, while DNA was identified as the major contributor to reduced T-cell growth and viability during TCR engineering process. Of note, the knock-in/knock-out (KI/KO) data demonstrated that the most optimized electroporation program could reach upwards of 85-90% KO and 50% KI/KO efficiencies (FIG. 1E), with no nonspecific KI/KO populations from RNP only, no-RNP, or no-DNA arms.

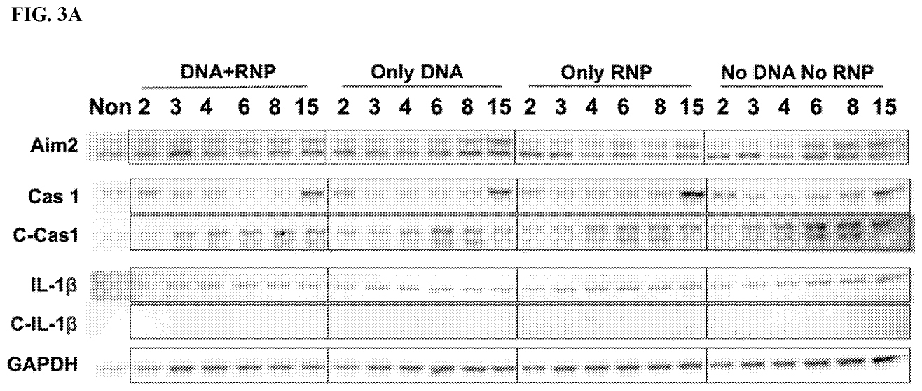

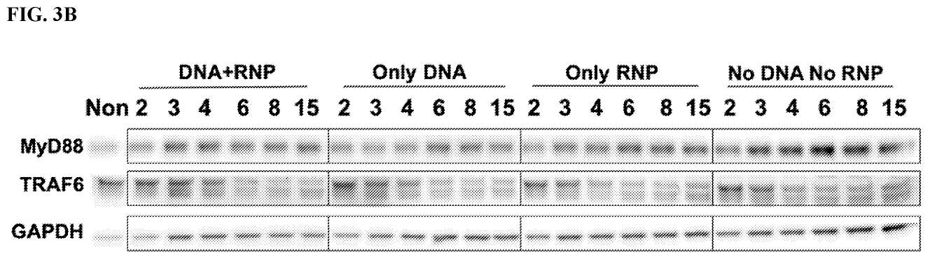

Based on what was reported with monocytes [4,5], we hypothesized that genotoxic stress post transfection, due to the presence of plasmid DNA in the T cells, triggers a viability drop and reduced culture expansion. To confirm this, cell samples from day 2 to day 15 were subjected to Western blot analysis and indeed both DNA only and DNA+RNP groups activated the cGAS-STING pathways as evident by the phosphorylation of STING, TBK1 and IRF3 accordingly (FIG. 1F). The RNP only group, despite achieving a high knock-out percentage (FIG. 1E), did not activate the cGAS-STING pathway (FIG. 1F). To rule out the possibility of other potential pathways that might impact culture growth or expansion in the presence of single stranded DNA (ssDNA) or double stranded DNA (dsDNA), we investigated activation of the inflammasome related pathway (Aim2/IL-1B) and the DNA endosome activation pathway (TLR9/MyD88) in T cells post transfection. Our findings showed that neither of these pathways were activated (FIGS. 3A, 3B), outlining the differences between the viral-based and nanoparticle-based DNA delivery approaches [6,7,8].

Pre-Treatment with BX795, AML and MRT Increases T Cell Viability, Expansion and Number of Total Edited Cells During TCR Engineering Process

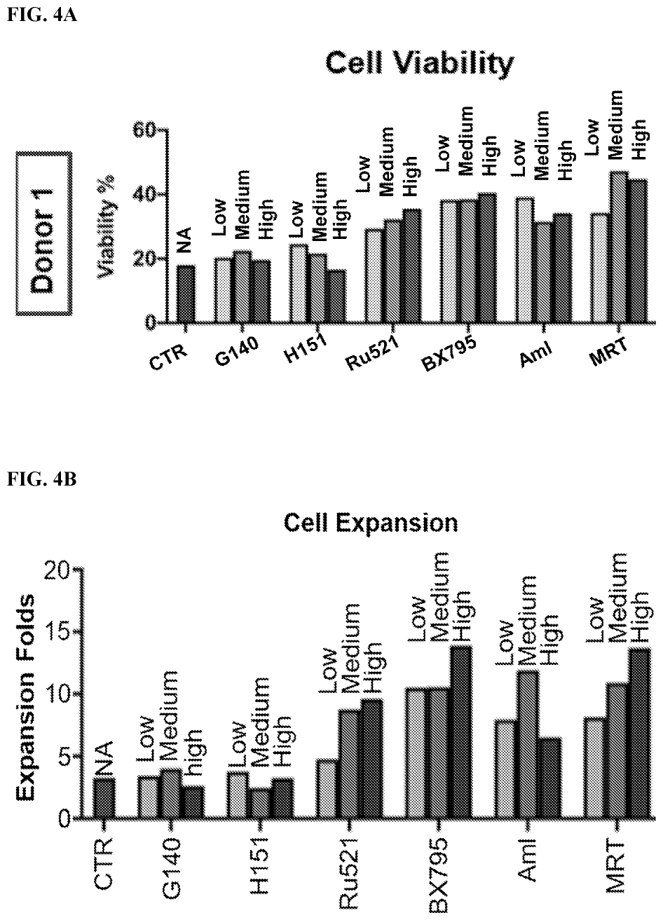

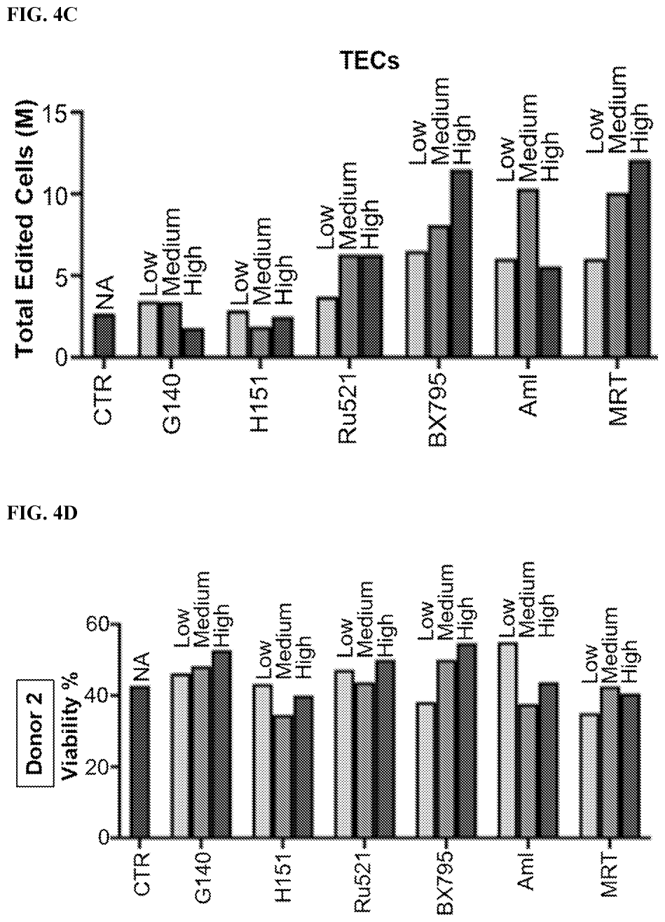

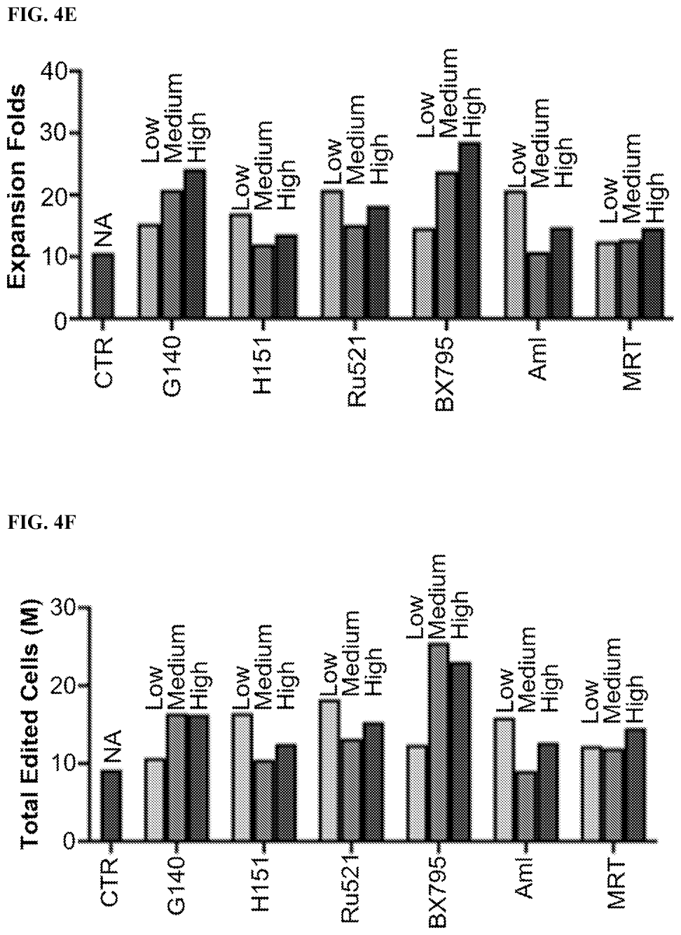

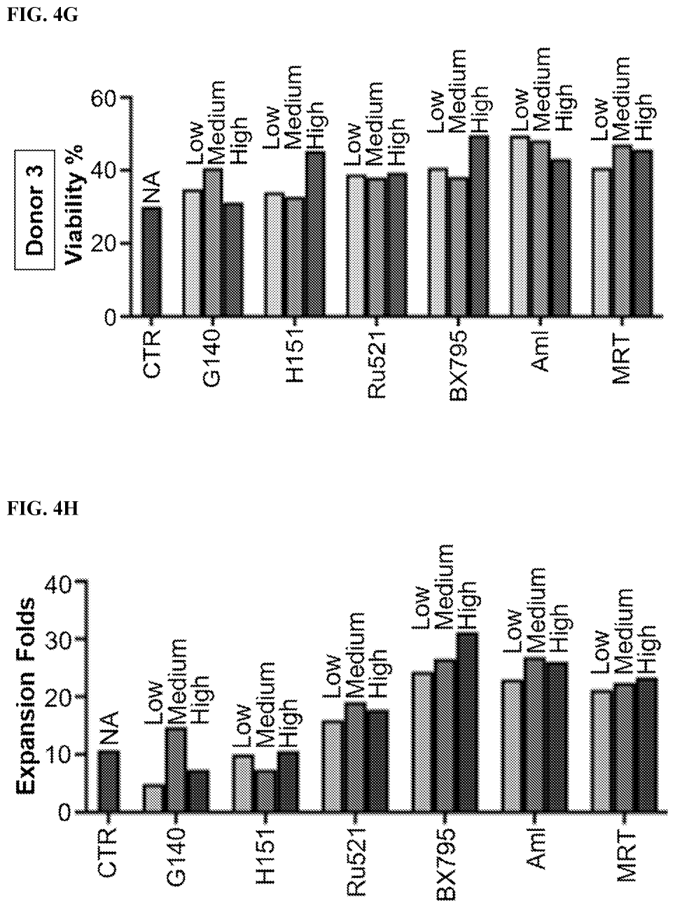

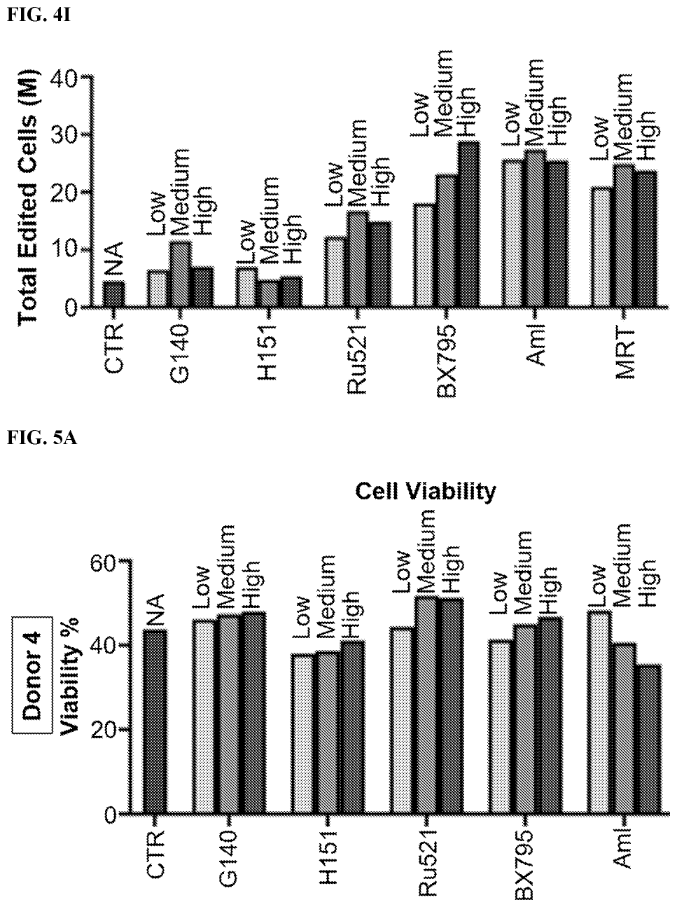

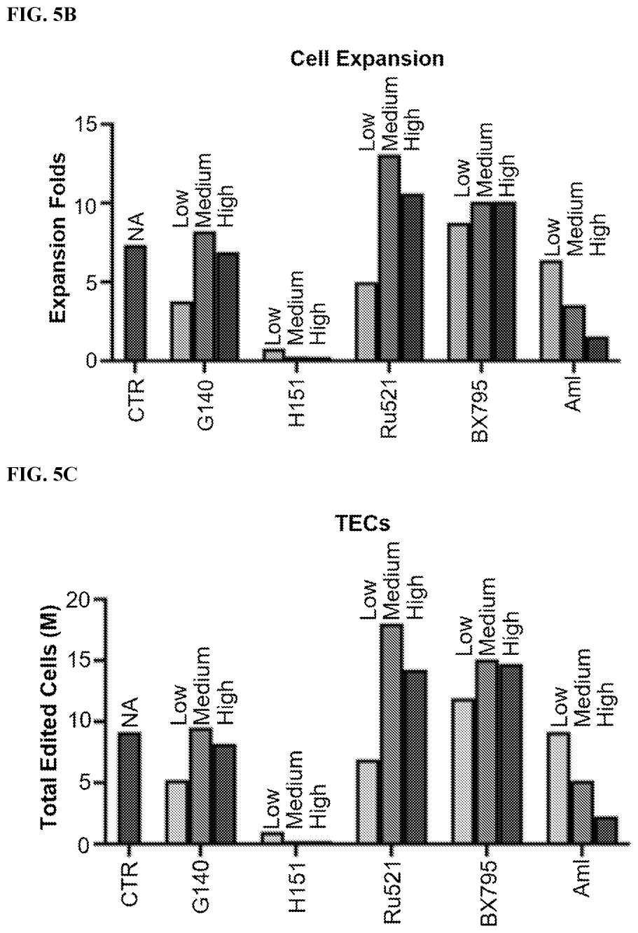

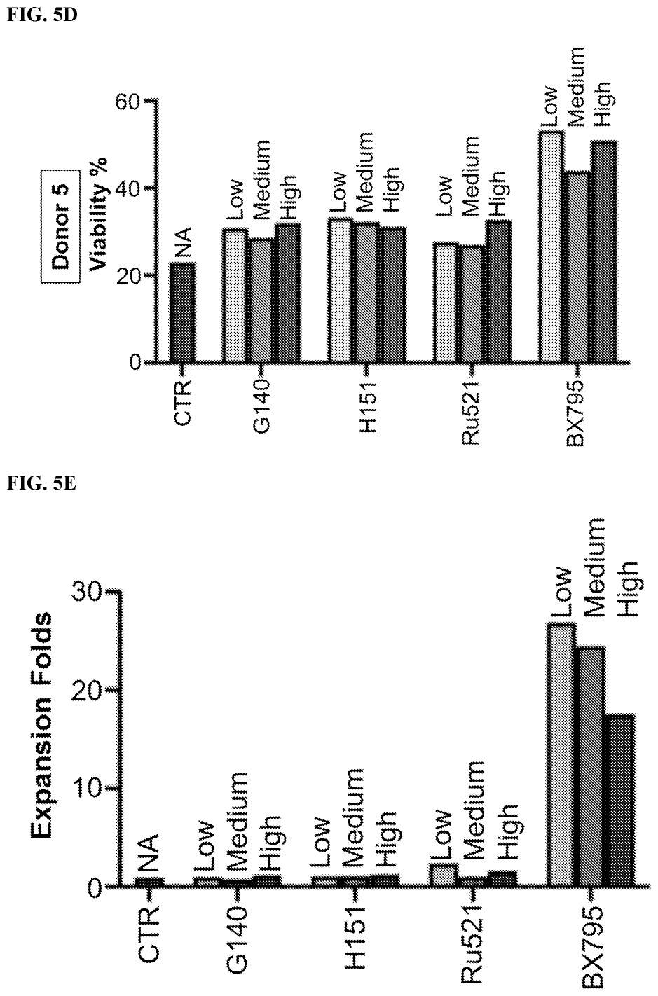

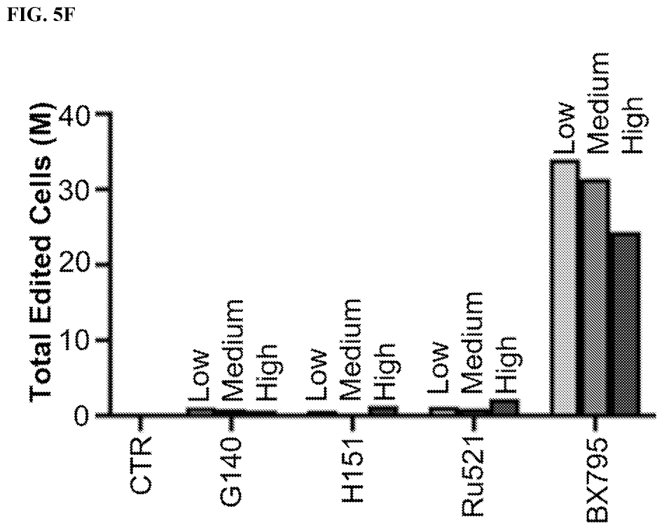

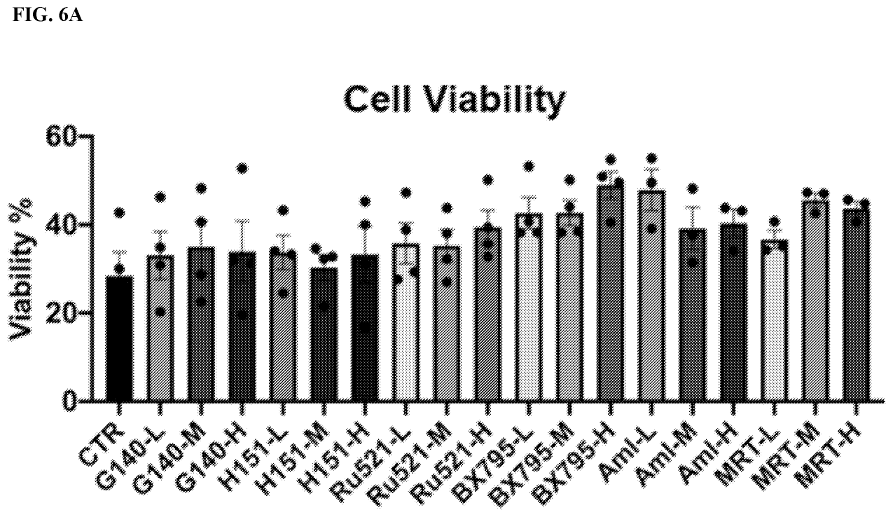

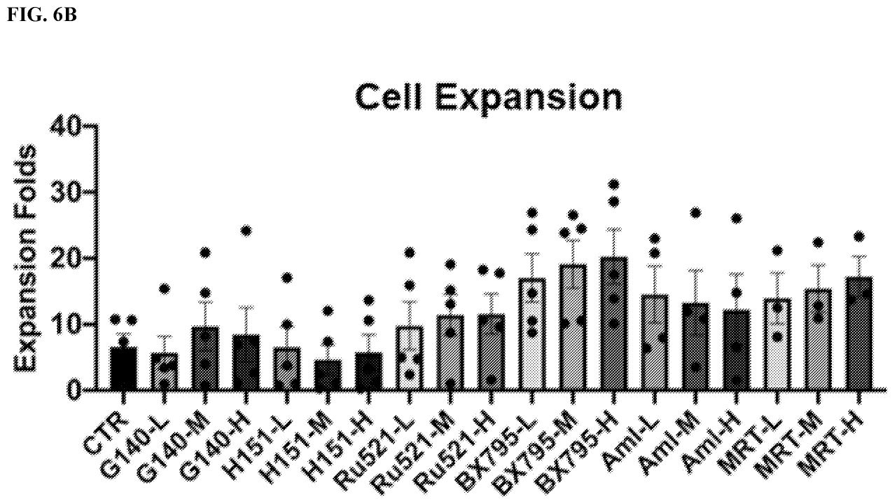

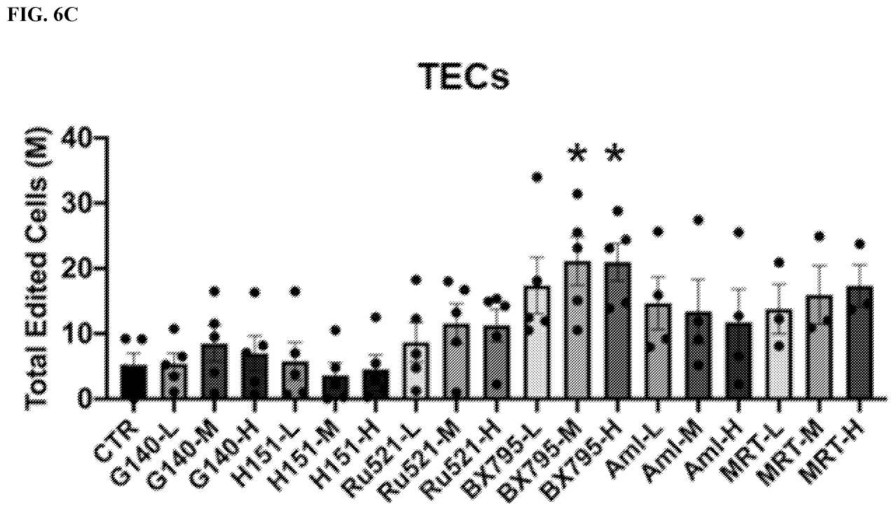

Since plasmid DNA appears to activate the cGAS-STING pathway, resulting in a viability drop and growth inhibition post electroporation in T cells, we investigated whether blocking activation of the cGAS-STING pathway can alleviate the negative impact of plasmid DNA on culture viability and growth. Six inhibitors targeting cGAS (G140, Ru521), STING (H151), and TBK1 (Amlexanox, BX795, MRT67307), were tested (Table 1). To bypass the potential contamination and non-specific immune response introduced by exposure to human serum, chemically defined culture medium with no serum was used in this study. T cells were treated with these inhibitors at the indicated concentrations (Table 1) and only prior to transfection for specific durations, since addition of inhibitors post-transfection negatively impacted culture growth. Pretreatment of T cells with some of these inhibitors, at optimized concentrations, significantly improved culture viability 48 h-post transfection (FIGS. 4A, 4D, 4G, FIGS. 5A, 5D). On average, culture viability was improved by about 2-fold compared to the untreated control group (CTR), when BX795 (5 uM), Aml (25 uM) and MRT (2.5 uM) inhibitors were used (FIG. 6A). In addition, final T cell product collected on day 15 exhibited higher cell expansion (FIG. 4B, 4E, 4H, FIG. 5B, 5E) compared to the control group, which had an average expansion of only 6.6-fold. Cells pre-treated with BX795 (5 uM), Aml (25 uM) and MRT (5 uM) reached 20.3, 14.5, and 17.2-fold expansion, respectively. On average, treatment of T cells with the aforementioned inhibitors (at the given concentrations), resulted in approximately 2-3 folds improvement in culture expansion and was consistent for all the donors (FIG. 6B).

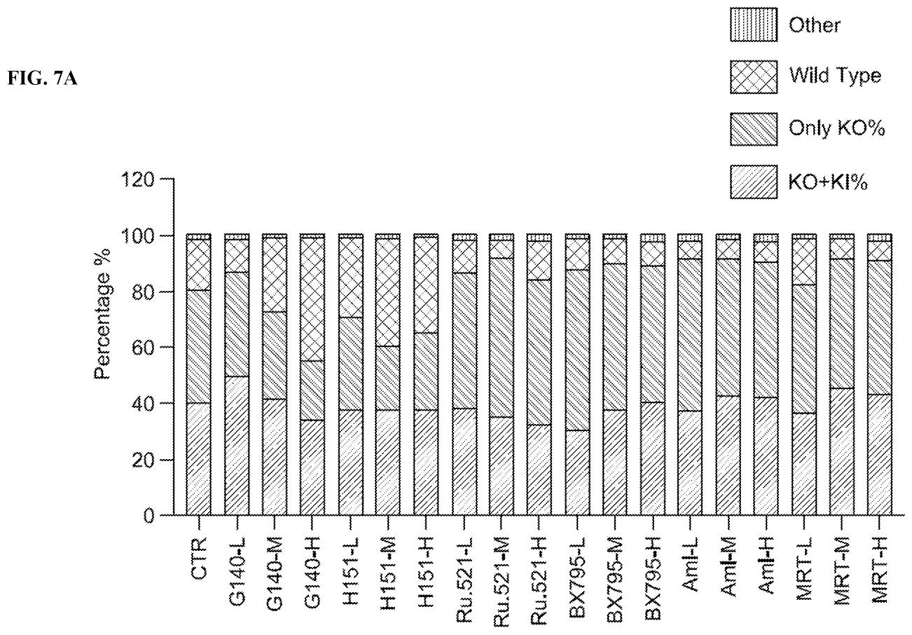

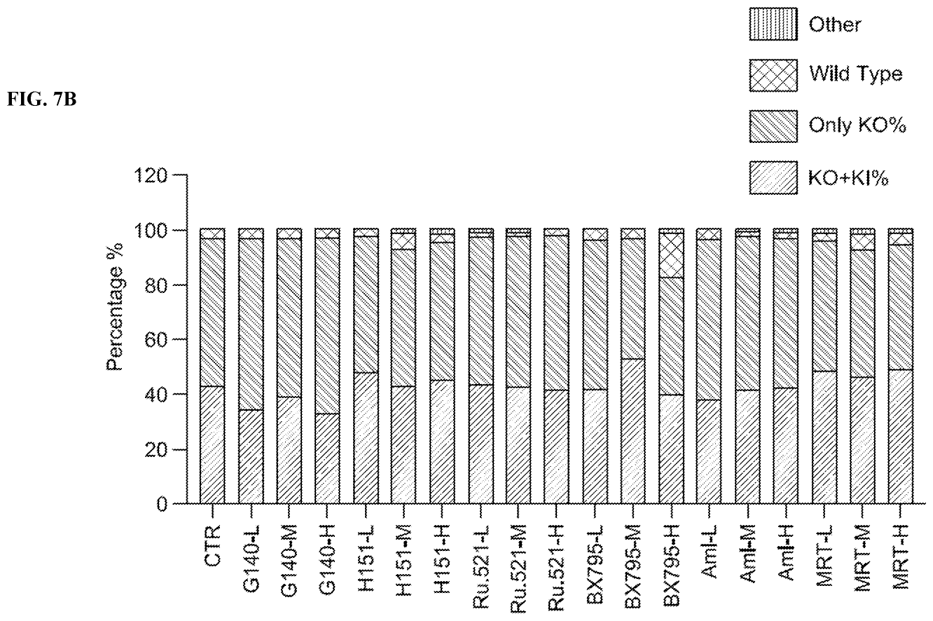

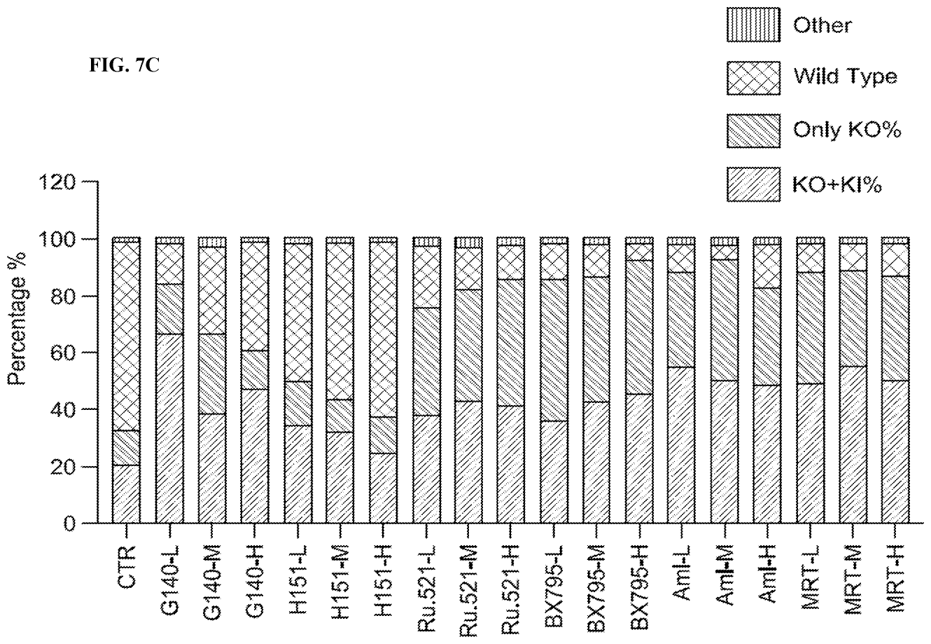

Since inhibition of cGAS-STING pathways should only mitigate plasmid DNA mediated genotoxicity, no impact on gene editing efficiency was expected and as such knock-in/knock-out (KO+KI) rates for TCR targeting were approximately comparable among all the donors (FIG. 7). The only exception was for the donors with poor cell health condition, where treatment of cultured cells with cGAS-STING pathway inhibitors improved KO+KI rates due to better survival and hence expansion of cells with KO+KI phenotype (FIG. 7C). This will be further discussed in the section below.

The total edited cell number (TECs) in the final drug product is arguably the most important metric for the adoptive cell transfer (ACT) and this, among a few other conditions, is considered one of the most important criteria for cell therapy purpose since it is expected to translate to the therapeutic impact in the clinic. Using the total cell number of the final cell product on day 15 multiplied by the percentage of knock-in, we get the TECs that are representative of our T cell engineering and culture output. With a starting seeding density of 2 million cells and without inhibitor treatment, as shown for the control conditions (CTR), the TEC numbers were approximately 5.24 million on average (FIG. 6C). However, when T cells were pre-treated with the indicated inhibitors, a significant increase in the output of TECs in all donors were observed (FIGS. 4C, 4F, 4I, FIGS. 5C, 5F), which was consistent with the cell expansion data. Pre-treatment with BX795 (2.5 uM), Aml (25 uM) and MRT (5 uM) increased the TEC number from 5.24 million to 21.1, 14.7, 17.4 million on average, respectively, contributing to an average increase of 303.4%, 267.0%, and 180% in TECs compared to the control group (FIG. 6C).

Due to the large donor to donor variations in T cell editing efficiency and expansion rates, use of statistical analysis in autologous T cell engineering studies is challenging. Interestingly, however, for T cells pre-treated with inhibitor BX795 at mid or high concentrations (2.5 uM and 5 um), TEC analysis revealed a statistically significant improvement compared to the control group (FIG. 6C), resulting in more than 3-fold improvement in TEC.

### Bx795 Pretreatment Improves Cell Viability and Expansion Rates by Inhibiting IRF3 Phosphorylation.

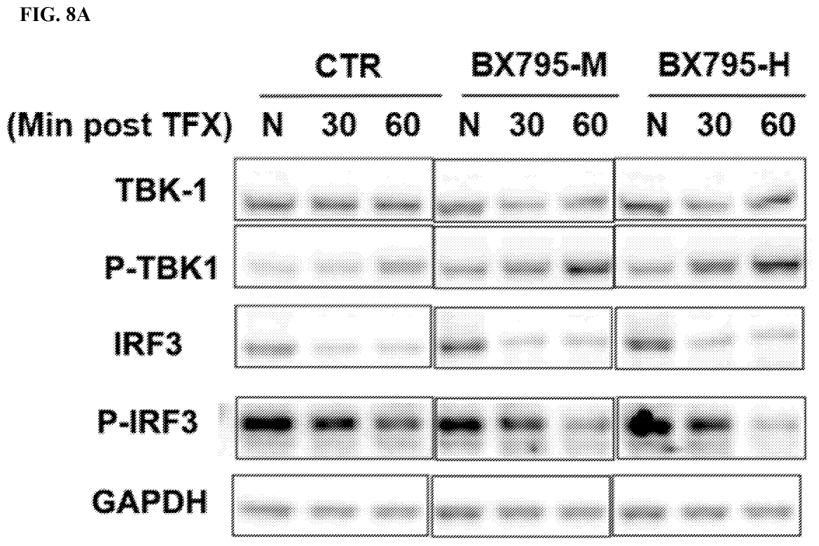

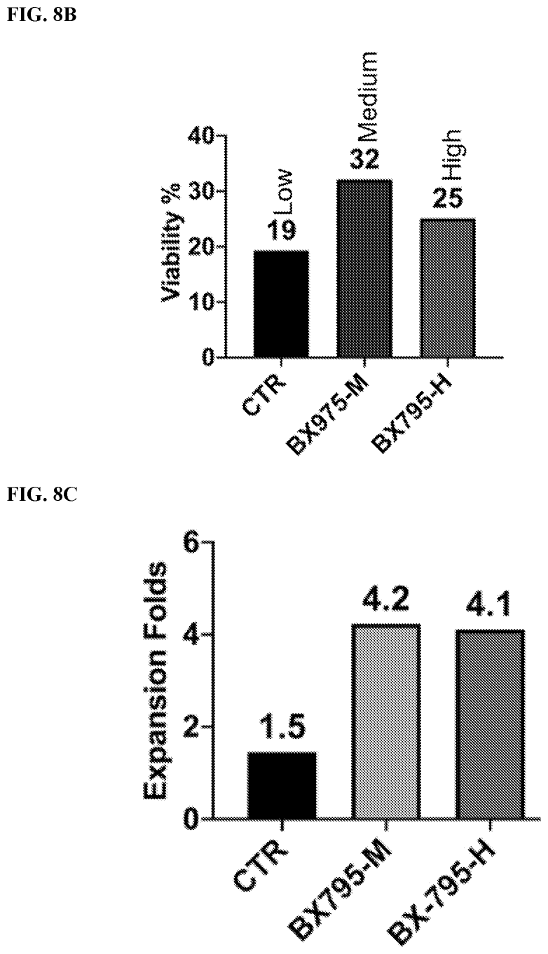

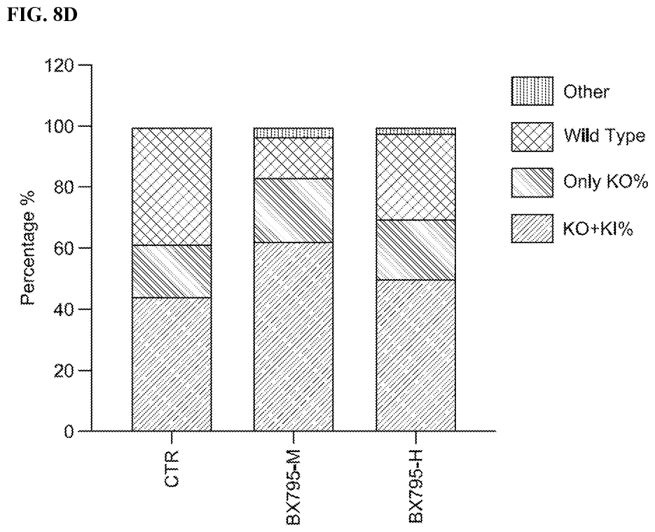

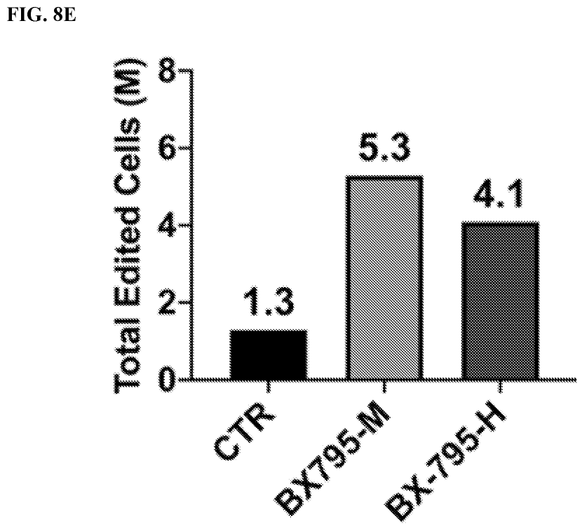

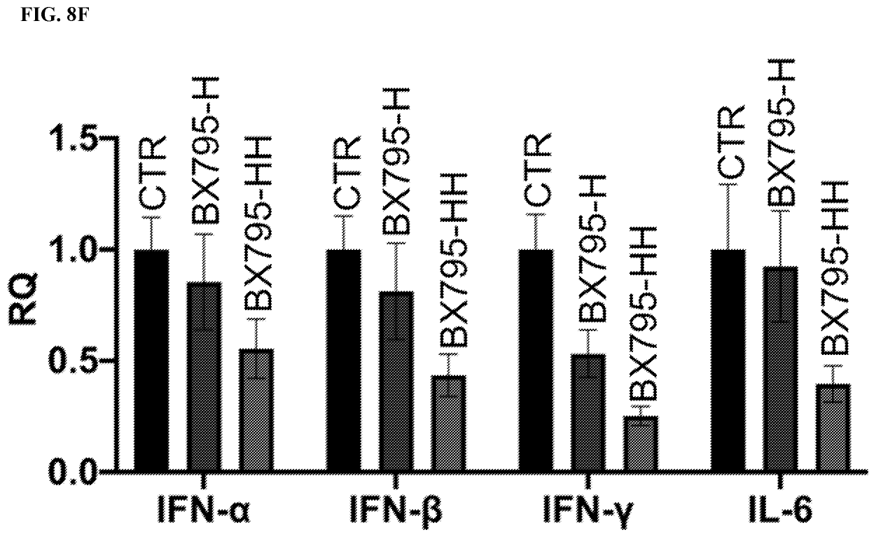

Based on the data, BX795 (at 2.5 uM and 5 uM) were the most effective in improving culture viability and growth and hence were used to conduct further studies. BX795 is a kinase inhibitor targeting TBK1, which is a kinase that functions downstream of the cGAS-STING pathway [9-11]. Western blot analysis revealed that pre-treatment with BX795 successfully attenuated phosphorylation of IRF3, which is the downstream target of TBK1, post transfection (FIG. 8A) at both medium and high concentrations. Consequently, the downstream cytokine expression, such as type 1 interferons (IFNa, IFNB) and proinflammatory cytokine (IL-6), were reduced upon pre-treatment with BX795 (FIG. 8F), indicative of the successful inhibition of the cGAS-STING pathways during the TCR engineering process. The TBK1 phosphorylation itself, however, was not attenuated and was actually enhanced post BX795 pre-treatment, consistent with a previous report where a 2-fold enhancement in TBK1 phosphorylation was reported upon BX795 treatment [9]. As previously shown (FIGS. 4A-4I and FIGS. 5A-5F), BX795 treatment resulted in higher % viability (FIG. 8B), increased T cell expansion rate (FIG. 8C), equal or better KI/KO ratio (FIG. 8E), and improved TEC numbers.

### Bx795 Inhibitor Treatment Improves Knock-Out and Knock-In Ratio in the Final T Cell Product.

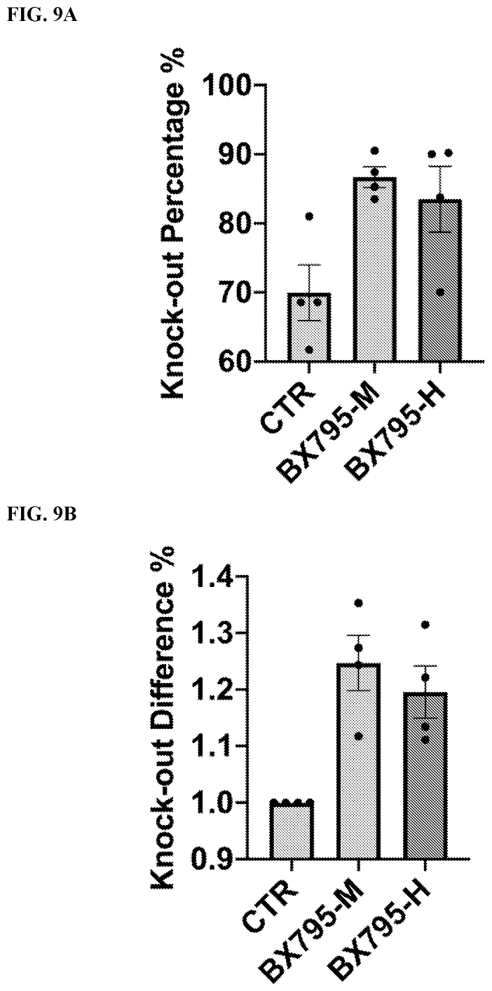

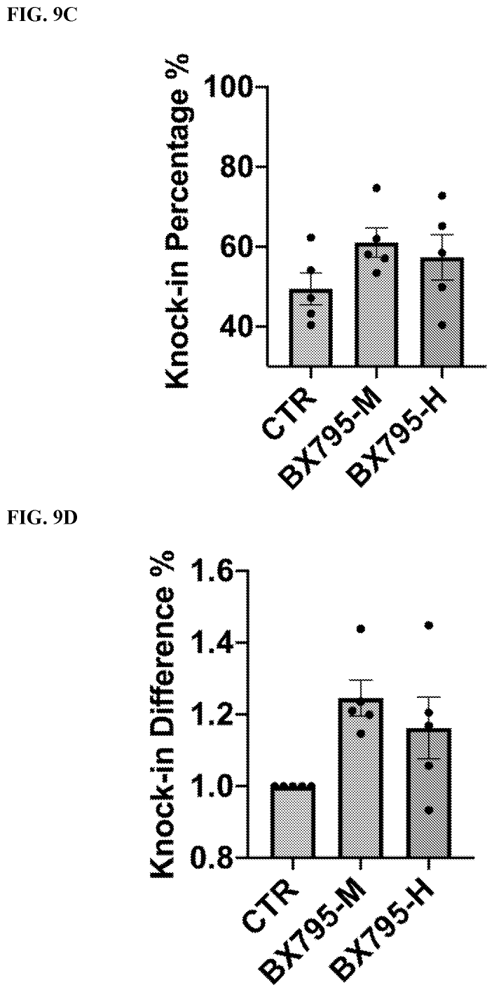

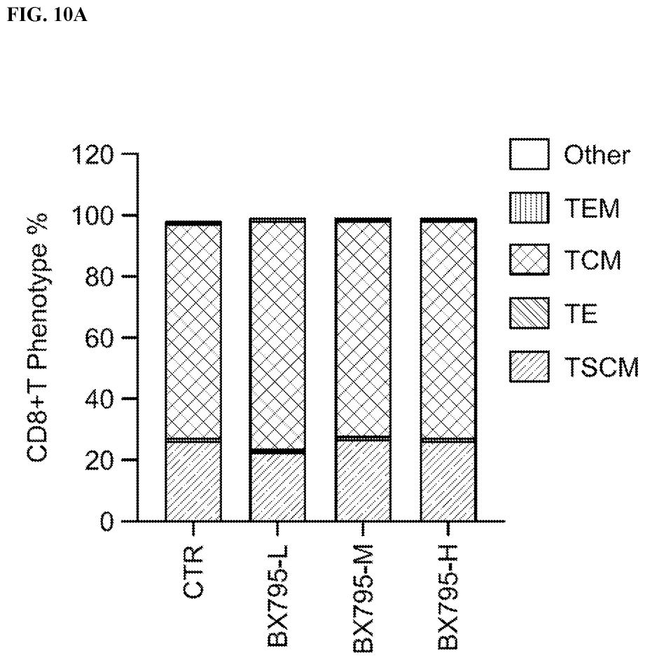

Pre-treatment of cultured T cells with BX795 inhibitor is not expected to have a negative impact on Cas9 protein mediated targeted cleavage of genes of interest, nor should it impact homology-directed repair processes. However, data from the final cell product shows that T cells pre-treated with BX795 had significantly higher total knock-out percentage than the control group (FIG. 9A). BX795 pre-treated T cells reached 86.7% (BX795-M) and 83.5% (BX795-H) knock-out efficiency compared to the control group with about 70% KO efficiency, with statistically significant higher editing values (FIG. 9B). Of note, BX795 pre-treatment of T cells also show higher knock-in percentage in the final cell product, increasing the knock-in ratio from 49.5% (CTR) to 61% (BX795-M) (FIG. 9C), which is statistically significant compared with non-inhibitor-treated control group (FIG. 9D). It is unlikely that pre-treatment of T cells with BX795 inhibitor could enhance Cas9 protein mediated cleavage of the target gene(s) or improve the plasmid DNA based homology-directed repair process. The observed improvements in knock-in and knock-out ratio in the final T cell product (FIG. 4C, 4F, 4I, 5C, 5F, 6C) is likely due to the survival and growth of the edited T cell as the observed improvements in TECs trends with expansion fold and higher viabilities in cultures pretreated with BX795 (FIG. 6C). T cells with successful KO+KI ratios might have lower potential for survival and expansion due to shifts in metabolism and higher cellular stress and pretreating T cells with BX795 could partially alleviate some of these cellular stresses.

Bx795 Inhibitor Treatment does not Affect Memory T Cell Phenotype.

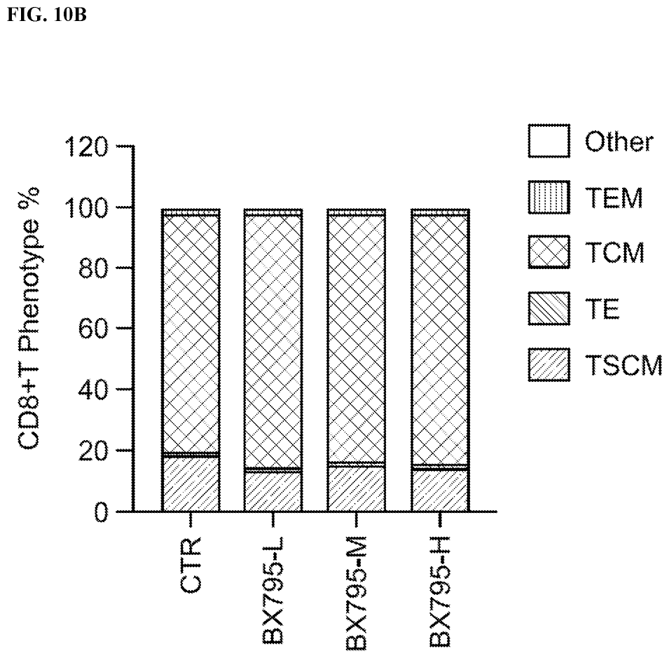

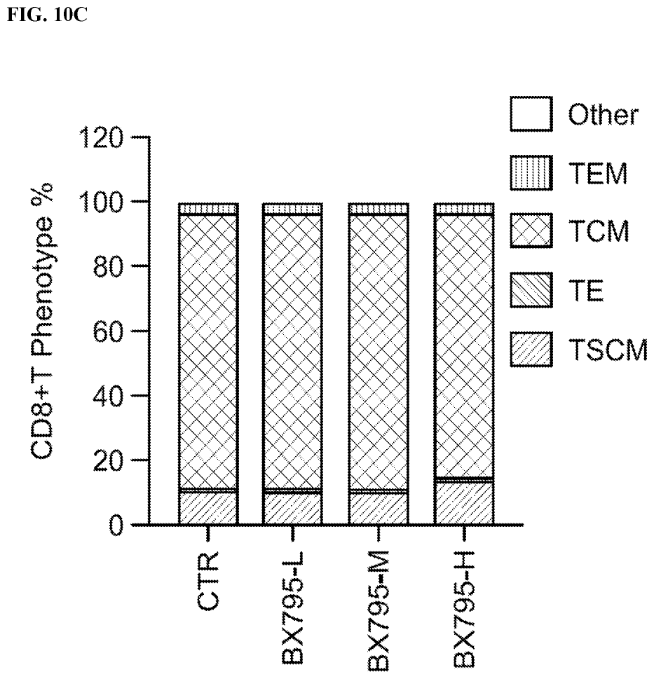

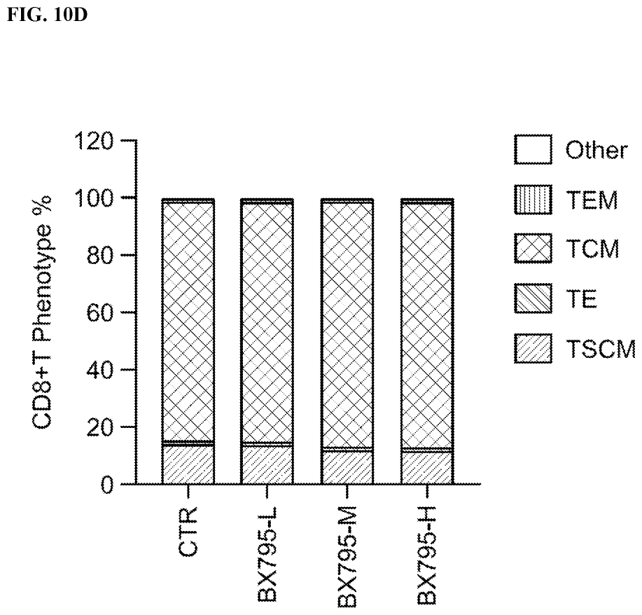

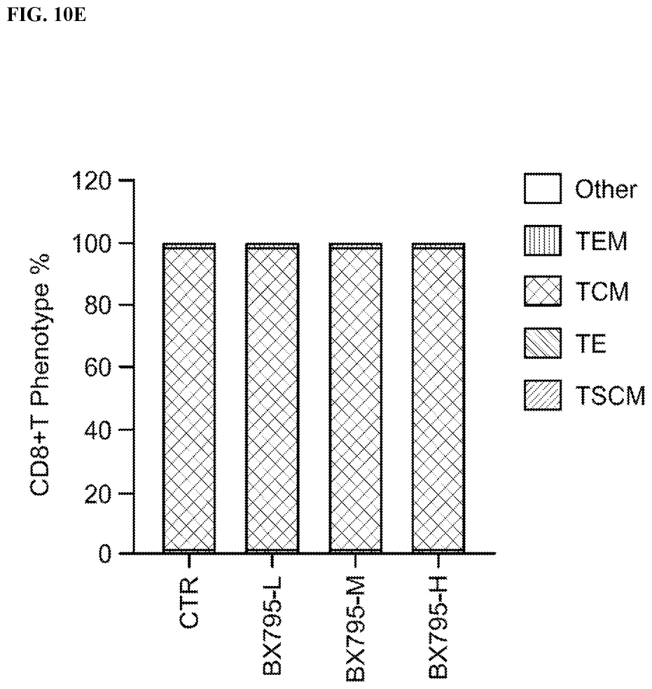

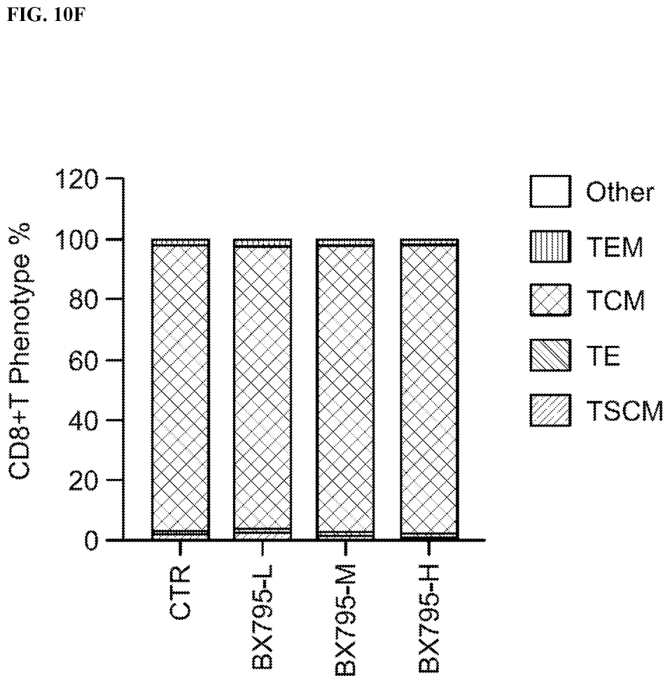

It has been anticipated that the number of memory T cells in the final T cell product strongly correlates with the clinic efficacy during T cell therapy as memory T cells have the potential for proliferation and long-term survival once reintroduced back to the patient [14,15]. The glycolysis to fatty acid oxidation metabolism balance in T cells, as well as the growth-related signaling pathways (etc. Jak-STAT pathway), can impact the T cell phenotype [16]. In our optimized T cell activation and culturing method, upwards of 95% of the cells have either central memory T cell (TCM) or stem cell memory T cell (TSCM) phenotype in the final cell product (FIGS. 10A-10F). The high memory T cell phenotype percentage potentially promotes higher proliferation and enhances tumor killing ability of the T cells. Pre-treating T cell cultures with BX795 inhibitor did not adversely affect T cell phenotype compared with the untreated control groups for different donors (FIGS. 10A-10F). This is a very important criteria for inhibitor selection/application since higher growth rates observed in inhibitor treatment groups could potentially change cellular metabolism, cytokine concentrations, and levels of metabolites in the culture medium, resulting in a change in T cell phenotype. Fortunately, BX795 treatment had no impact on T cell phenotype.

CD8+ T Cells Derived from Cultures Treated with BX795 Inhibitor Exhibit Comparable Activation, Proliferation and Target Cell Killing to the Untreated Control Group.

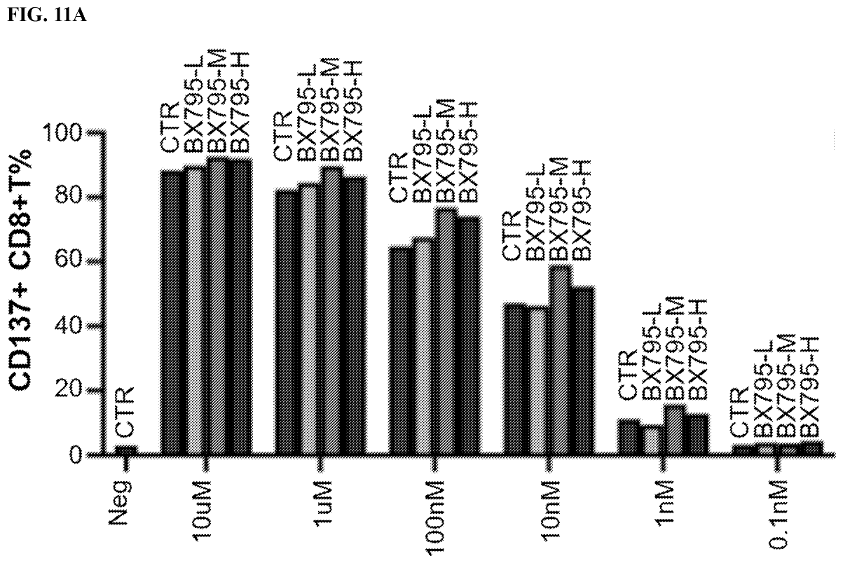

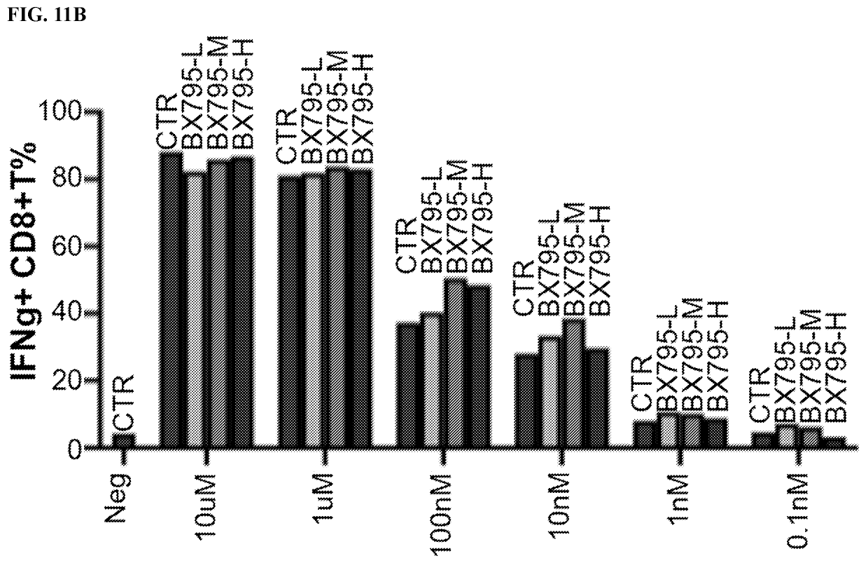

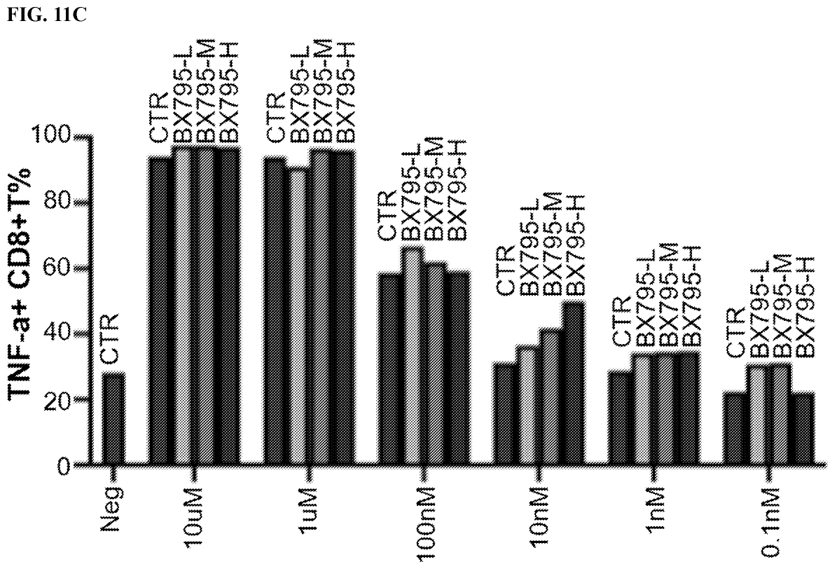

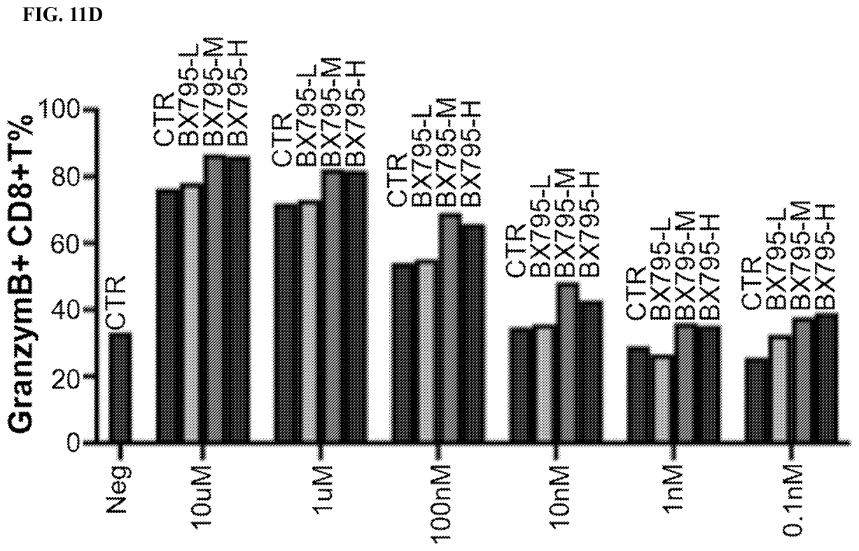

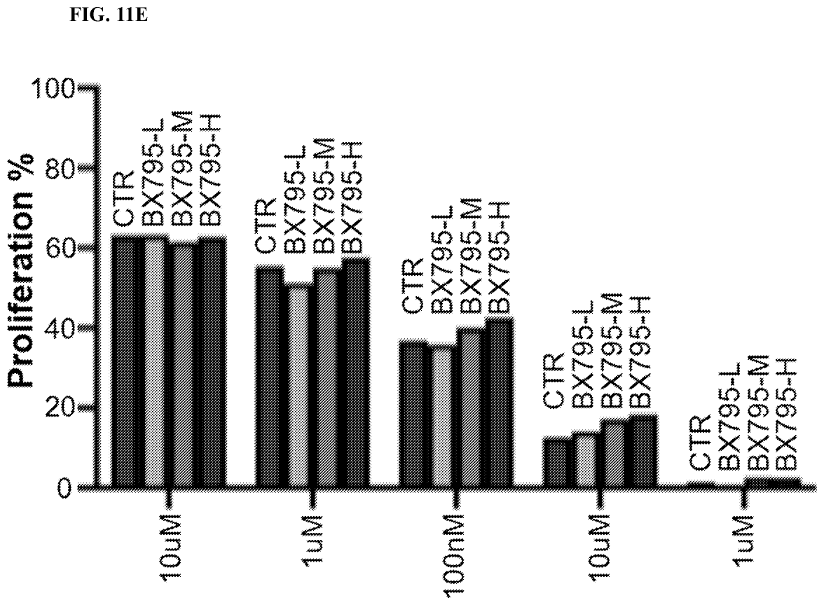

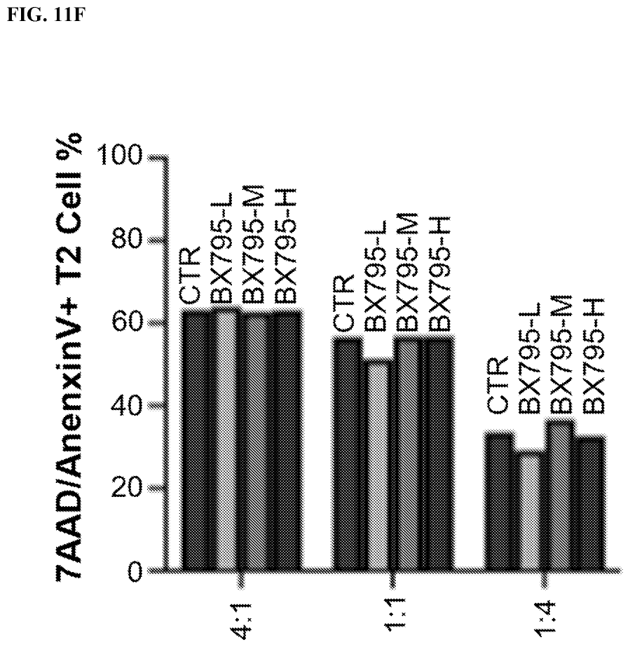

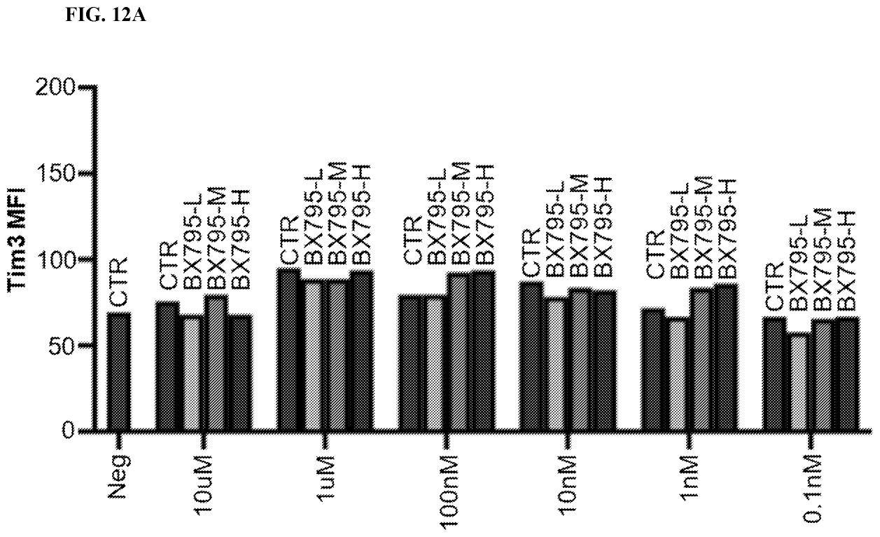

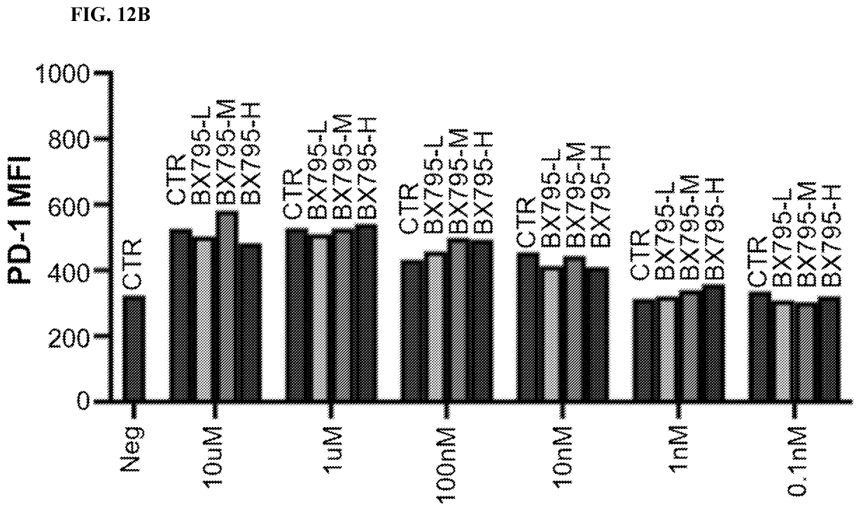

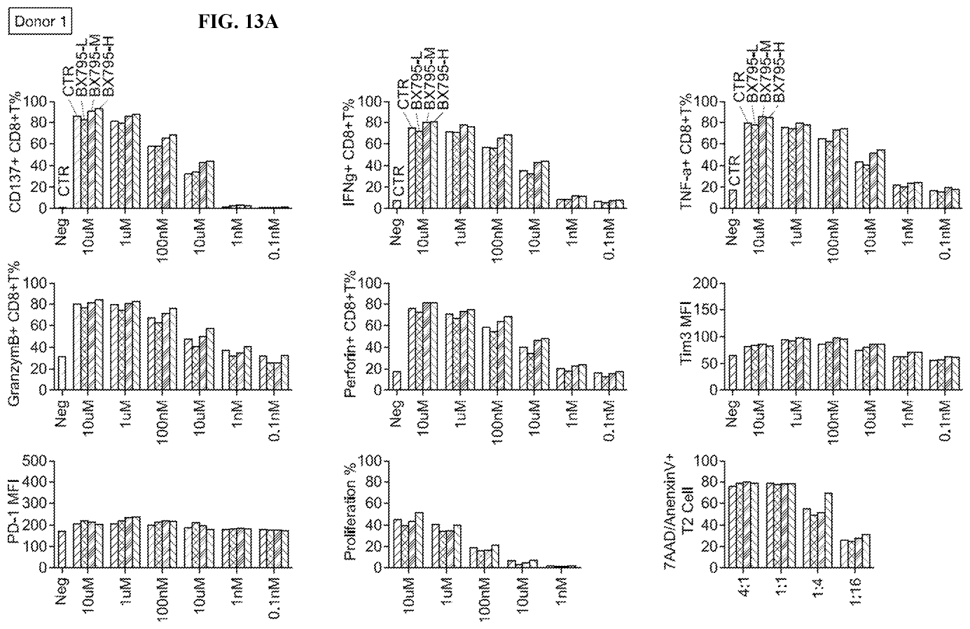

The cGAS-STING pathway is crucial in detecting and neutralizing genotoxic stress. It is a main component of innate immune system that can trigger inflammation in response to cytosolic DNA detection [17, 18]. By blocking cGAS-STING pathways, we observed a significant improvement in culture viability, cell expansion rate, and hence total edited cell number. To ensure that treating cells with BX795 does not have any adverse impact on the function of T cell product, we used cells from four independent donors and followed activation, transfection, and culturing protocols (as described). Harvested T cells from inhibitor treated and control groups were pulsed for 24 h with indicated concentrations of a specific peptide (WT1), which binds to the engineered TCR. Flow cytometry data showed that T cells pre-treated with BX795 had a comparable activation profile as the untreated control cells with regards to CD137 (FIG. 11A), IFNg (FIG. 11B), TNFa (FIG. 11C), and Granzyme B expression (FIG. 11D). The data indicate that pretreating T cells with BX795 inhibitor at the indicated concentrations did not affect T cell activation and cytokine expression. Another major concern is the exhaustion of the engineered T cell cultures. To measure levels of T cell exhaustion, the expression of exhaustion markers, such as Tim3 and PD-1, was evaluated. No significant differences in levels of T cell exhaustion markers were observed in the inhibitor treated or untreated groups in medium or low peptide concentration, compared with negative control (FIGS. 12A, 12B). As expected, T cells pretreated with inhibitors had comparable levels of Tim3 and PD-1 expression to the untreated control group (FIGS. 12A, 12B).

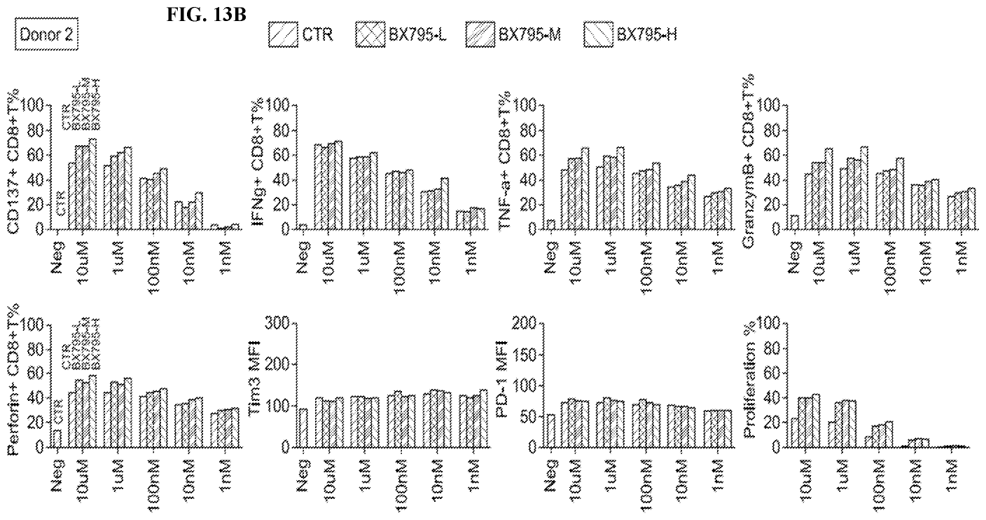

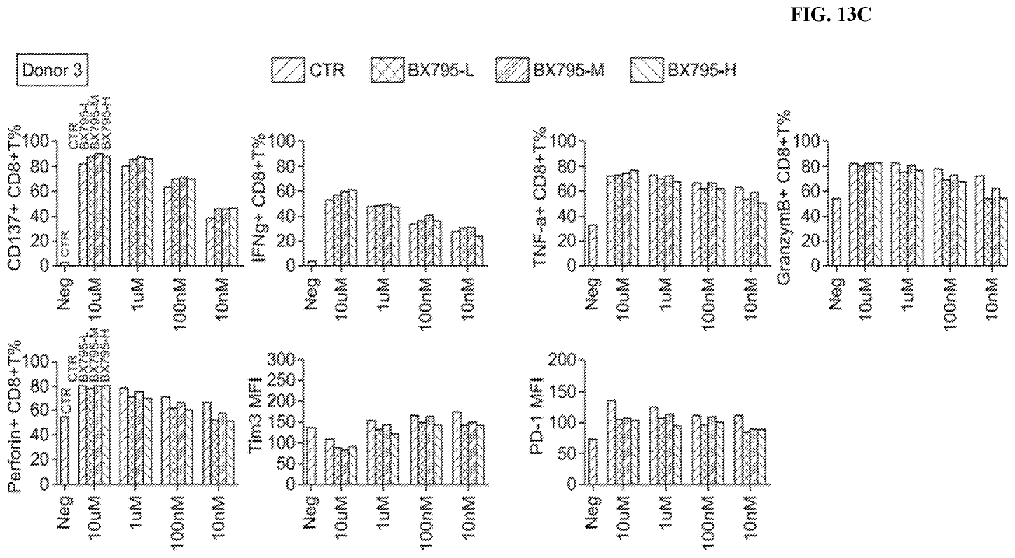

We next investigated proliferation of T cell after treatment with BX795. The final T cell product obtained from BX795 pre-treated or untreated groups were labeled with CFSE far-red, and co-cultured with WT1 peptide at the indicated concentrations for 72 h. T cell proliferations in response to antigen specific stimulation, as analyzed by flow cytometry, were comparable between the BX795 treated and untreated samples (FIG. 11E). T cells treated with higher concentration of WT1 peptide achieved high (˜60%) proliferation rates, implying the strong and antigen specific proliferation of the final T cell product. Finally, we compared T cells isolated from BX795 pre-treated as well as untreated control cultures in a target cell killing assay. Target cells T2 (T) were labeled with CFSE-far red, and then pulsed with 20 uM WT1 peptide before co-incubation with our final T cell product (E) at a ratio dependent manner. Our data confirmed that T cells pretreated with inhibitors had comparable target cell killing ability to that of untreated control groups in all E:T ratios (FIG. 11F). Of note, compared with the CTLs raised from conventional vaccine strategies, our T cells achieved high killing efficiency with much lower E:T ratio [19, 20], outlining the potential advantage of antigen-specific tumor cell killing of our TCR engineered T cells. Taken together, our data has demonstrated the safety and feasibility of using BX795 cGAS-STING pathway inhibitor for T cell engineering process, outlining the potentially broad impact of such inhibitors in the adoptive T cell therapy field. Use of cGAS-STING inhibitors specifically improved T cell engineering efficiency, cell expansion rates, and hence total edited cell numbers. By improving T cell viability and growth post electroporation, two to three folds higher TECs with comparable activation, proliferation and target-cell killing ability to the control group could be obtained. Of note, we showed that this phenomenon is not donor specific or restricted to certain antigens, as similar favorable results were observed for T cells obtained from all donors in this study (FIGS. 4A-4I, FIGS. 5A-5F, FIG. 13A-13C).

### DISCUSSION

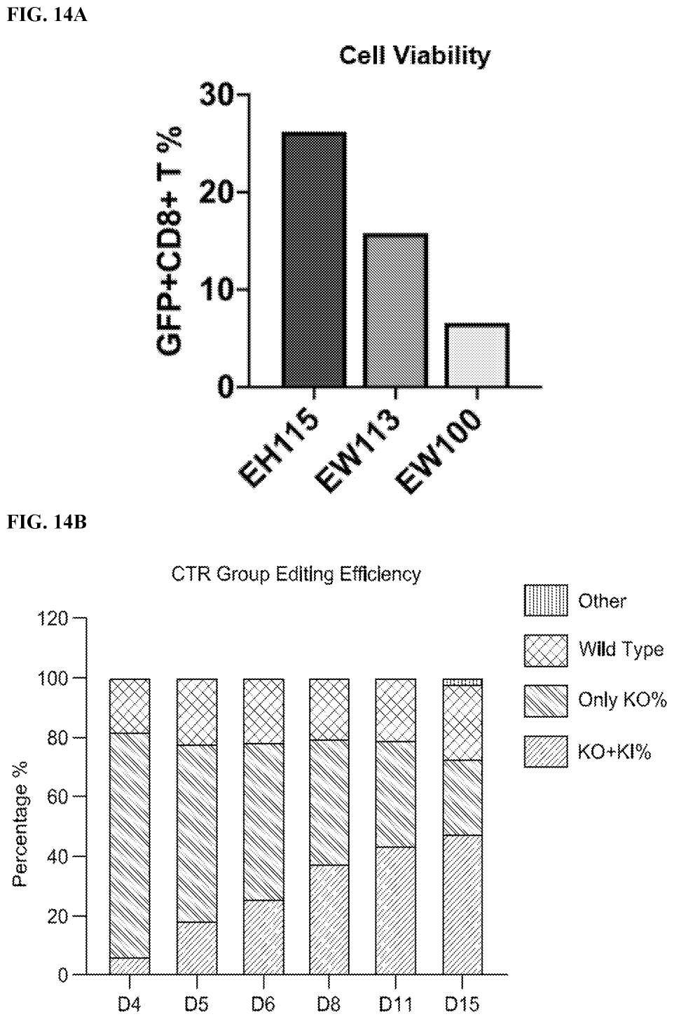

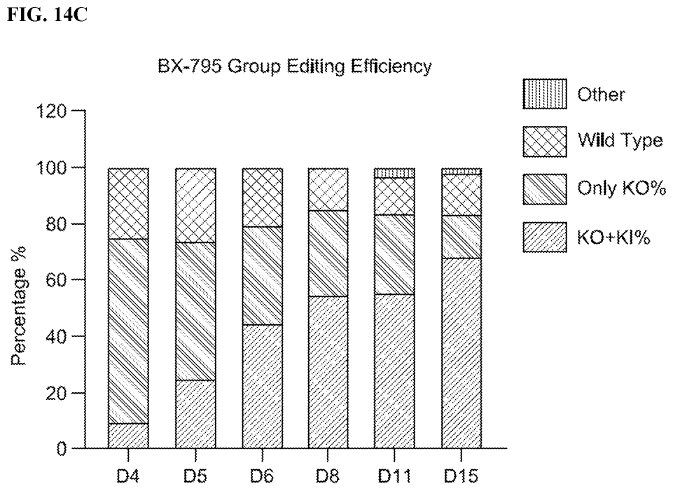

In this study, we have introduced an easy, efficient and manufactory friendly solution to improve T cell viability and growth during TCR engineering process. Pre-treating T cells with TBK1 inhibitors resulted in higher T cell expansion, and improved TCR editing efficiency without impacting the expected phenotype(s) of T cell final drug product (FDP). We successfully performed TCR engineering by electroporation RNP and long DNA sequence into T cells, and at the same time improved editing efficiency, electroporation scale and cell expansion rates. Our data reveals a direct correlation between electroporation program (pulse code) used and T cell viability and expansion rates. We hypothesized that this phenomenon is related to the amount of DNA introduced to T cells during the electroporation process where the stronger pulse code (for example, EH115) results in delivery of more plasmid DNA constructs to T cells compared to the milder pulse code (for example, EW100). We verified this by using a GFP plasmid construct during T cell transfection (FIG. 14A). This also explains why use of stronger pulse codes results in reduced viability as our data suggests plasmid DNA is the main cause of cell death and growth reduction post electroporation. When using TBK1 inhibitors, we observed that better T cell expansion can be detected in multiple pulse code (EW113, EH115 etc.), Additionally, the BX795 inhibitor performs better in EH115 groups, likely due to introduction of more plasmid DNA, which triggers higher genotoxicity levels.

To the best of our knowledge, this is the first report showing that TBK1 inhibitor BX795 enhances TCR editing efficacy in a non-viral cell editing approach. Since BX795 has not been reported to directly impact homology-directed DNA repair (HDR) or DNA internalization process, knock-in rate improvements mediated by BX795 pre-treatment is not likely due to the dsDNA repairing enhancement. Though some literature indicate a potential connection between cGAS pathway and HDR function, no clear mechanism for is claim has been delineated. We hypothesized that BX795 enhances survival of edited T cells during both electroporation and subsequent culturing post transfection by reducing genotoxicity. This claim is partially supported by time-gradient TCR-dextramer flow cytometry analysis (FIG. 14B, 14C) showing that BX795 treatment increases exogenous TCR expression both early on (day 4, 5) and throughout the culture process (days 6 to 15), compared to the control group, as well as higher increase curve from.

The mechanism of genotoxic stress is complicated. Though our western blot and qPCR data shows a considerable reduction in cGAS/STING pathway activation post BX795 treatment (FIG. 8A), we suspect that plasmid DNA mediated genotoxic stress is not fully resolved by BX795 treatment. Levels of type 1 interferons, such as TNFα and TNFβ, did not show a significant decrease upon BX795 treatment, likely due to removal of the BX795 inhibitor prior to transfection. Exposing T cells to TBK1 inhibitors both before and after electroporation, or prolonging the treatment time might solve this problem.

T cell culture proliferation post-transfection can be significantly enhanced by CD3/CD28 stimulation or exposure to TCR specific peptide antigen displayed on MHC-I molecules. However, this inadvertently results in T cell exhaustion as well as high percentage of effector T cells in FDP. Previous studies in CAR-T research indicated that memory phenotype CD8+ T cells (TCM, TSCM, etc.) maintain better circulation time, proliferation potential, and target tumor cell killing ability when reinfusing back to patients. In this study, we used IL-7/IL-15 cytokines to enhance promote memory CD8+ T cell generation, resulting in more than 95% memory TCM or TSCM in FDP (FIGS. 10A-10F). However, this negatively affected T cell growth rates where on average only 5.24 folds T cell expansion could be achieved. Pretreating these T cell cultures, prior to electroporation, with TBK1 inhibitors significantly improved T cell cultures expansion rates up to 20.3-fold (FIGS. 4A-4I, FIGS. 5A-5F, FIGS. 6A-6C), with no detectable adverse impact on T cell phenotype.

Undoubtedly, non-viral genome targeting approaches have the potential to reduce cost, improve safety, and shorten timelines for developing next generation cell based immunotherapies. Advancements in DNA synthesis and next generation sequencing techniques enable synthesis of gRNA and DNA plasmid template within days. However, use of electroporation as the most efficient non-viral gene delivery/editing approach, requires overcoming a variety of burdens such as harsh impact on cultured cells and plasmid DNA mediated genotoxic stress. In that regard, many parameters such as electroporation instruments, buffers, and electroporation programs need to be tested and optimized in order to find a balance between knock-in efficiency, cell expansion and the overall T cell phenotype. Our work introduces a new approach to effectively address the genotoxic stress response in an easy and cost effective manner in T cells from different donors. Treating T cell cultures with cGAS inhibitors prior to transfection can improve cell viability, promote cell growth, and enhance editing efficiency. Our process is scalable, cost effective, and applicable to not only T cell engineering but any non-viral cell engineering process as well.

### Methods

### Rna Ribonucleoprotein and DNA Plasmid

Single guide RNA sequences for both TRAC and TRBC were derived as described by Oh, Senger et al and ordered from Synthego (Menlo Park, CA, USA). SpyFi Cas9 protein was purchased from Aldevron (Fargo, ND, USA) and used at a final concentration of 0.05 mg/ml for electroporation. For 100 ul electroporation procedure, 2.5 ug of Cas9 protein was pre-complexed with a 3-molar excess of sgRNA for each knock-out site then mixed prior to delivery. Nanoplasmid with WT1 TCR sequence was ordered from Nature Technologies (Lincoln, NE, USA) and was used at a working concentration 150 ug/ml in electroporation.

### Cell Isolation and Activation

Peripheral blood mononuclear cells (PBMCs) were isolated from human cryopreserved leukopak using Ficoll gradient centrifugation (400 rmp, 25 min). All samples were obtained from healthy donors genotyped with HLA A*02:01 MHC class I complex. Then CD8+ T cells were isolated using the Miltenyi AutoMACS cell separator according to the manufacturer's protocol. Isolated CD8+ T cells were then mixed with Miltenyi T cell TransACT reagent (1:100) (Catalog #130-111-160), 25 ng/mL IL-7 (Catalog #130-095-367), and 50 ng/mL IL-15 (Catalog #130-095-760) in Fuji Prime-XV medium (IrvineScientific Catalog #91154) for 48 h for activation.

### Electroporation

Cells were electroporated with the 4D-Nucleofector System (Lonza) according to the manufacturer's protocol. After activation and inhibitor treatment, 10×10⁶ (or 10 million) CD8+ T cells were washed and resuspended with Lonza P3 primary cell nucleofector solution (Catalog #V4XP-3024) and then mixed with pre-mixed RNP and DNA plasmid before transferring to Lonza 100-uL cuvette. After electroporation, 400 ul of FUJI Prime-XV medium was added to the cuvette and incubated for 15 min before seeding cells in culture flask. Pulse code used in this manuscript is EH115 for all circumstances.

### Cell Culture

All inhibitors were purchased from invivogen. Unless stated otherwise, cells were cultured with Fuji Prime-XV medium with 25 ng/ml IL-7 and 50 ng/ml IL-15 (Complete medium). After electroporation, T cells were carefully transferred to 24-well gREX (Wilsonwolf, Catalog: 80192M) at 1-2 million cells/cm² seeding density. 8 ml of complete medium were added per well and 50% of the medium was exchanged 6 days later. Cells were incubated at 37° C. and 5% CO2. The NucleoCounter NC-200 automated cell counter with the Via2-Casette were used in this study for cell counting and viability analysis.

### Flow Cytometry

All surface and intracellular staining antibodies were purchased from Biolegend and BD Biosciences as listed in Table 2. Dextramer antibodies for knock-in analysis were purchased from Immudex (Catalog: WB3469-PE, WT1) and used following manufacturer's protocol. Surface staining, CFSE staining, 7-AAD/annexin V staining, and intracellular staining were performed as previously described [17]. Samples were analyzed using the BD FACSLyric flow cytometer (BD Biosciences) and FlowJo v10 software. For proliferation and cell killing assay, data were corrected by a negative control or target cell-only control.

### Western Blot

Antibodies for western blot were purchased from Cell Signaling and listed in Table 3. T cell pellets were collected, washed and stored as frozen pellets in −80° C. until use. Cell pellet processing, western blot experiments and data analysis were performed as previously described (Tang, J B C, 2020).

qPCR

RNeasy Mini Kit (Cat. #74106) was purchased from Qiagen. TaqMan RNA-to-CT 1-Step Kit (Cat. #4392938) and Taqman primer-probe assays with reporter dye FAM and MGB quenchers were purchased from Thermo Fisher (assay information listed in Table 4). After RNA isolation, 2 ng RNA was mixed with Taqman primer-probe mix, TaqMan RNA-to-CT 1-Step master mix, and RT enzyme mix in a 10 uL qPCR system according to the manufacturer's protocol. Reaction readings were measured using QuantStudio 6 Flex machine and data was analyzed using QuantStudio Real-Time PCR software v1.2. PCR cycling conditions were 50° C. for 30 min, 95° C. for 10 min, 40× cycles of 95° C. for 15 s, 60° C. for 1 min. All data points were collected in triplicates, using RNA18S as internal control (sample ID: #Hs03928990_g1).

### Data Analysis and Statistics

Western blot data were analyzed with Image Lab 6.1 (Bio-Rad). Flow cytometry data were analyzed with FlowJo software v10 (BD Biosciences). Data from different assays were sorted in Excel (Microsoft). Graphs were created and presented with Graphpad Prism 10. For all statistical analysis, data has been presented as mean±SEM.

### Table 1. Inhibitor Treatment Concentration Used

Two days post activation, CD8+ T cells were washed and resuspended with FUJI Prime-XV complete medium containing inhibitors at the indicated concentrations for 6 h. After treatment, cells were washed and resuspended in Lonza P3 buffer prior to electroporation. Post transfection, T cells were cultured with FUJI Prime-XV complete medium without inhibitor.

| Inhibitors | Target | Low Dosage | | Mid Dosage | | High Dosage | |
|---|---|---|---|---|---|---|---|
| G140 | cGAS | 1 | uM | 2.5 | uM | 5 | uM |
| Ru.521 | cGAS | 1 | uM | 2.5 | uM | 5 | uM |
| H151 | STING | 1 | uM | 2.5 | uM | 5 | uM |
| BX795 | TBK-1 | 1 | uM | 2.5 | uM | 5 | uM |
| Amlexanox | TBK-1 | 25 | uM | 50 | uM | 100 | uM |
| MRT67307 | TBK-1 | 1 | uM | 2.5 | uM | 5 | uM |

TABLE 2
Flow Cytometry Antibody List

| Antibody Target | Fluorescence | Vendor Name | Catalog Number |
|---|---|---|---|
| CD3 | BV510 | BD Biosciences | 563109 |
| CD3 | APC | BD Biosciences | 340440 |
| CD8 | APC-Cy7 | BioLegend | 300926 |
| CD27 | PerCP-Cy5.5 | BioLegend | 302820 |
| CD45RO | BV421 | BioLegend | 304224 |
| CD45RA | PE | BioLegend | 304108 |
| CD62L | BV605 | BD Biosciences | 562720 |
| CD95 | PE-Cy7 | BD Biosciences | 561633 |
| CD137 | PE | BioLegend | 309804 |
| CD197 | AF700 | BD Biosciences | 561143 |
| 7-AAD | NA | BioLegend | 79993 |
| Annexin V | FITC | BioLegend | 640906 |
| DAPI | NA | Miltenyi Biotec | 130-111-570 |
| Granzyme B | BV421 | BioLegend | 396414 |
| IFN-γ | PE | BioLegend | 502509 |
| Perforin | Percp-Cy5.5 | BioLegend | 353314 |
| PD-1 | BV510 | BioLegend | 367424 |
| TCR-αβ | Percp-Cy5.5 | BioLegend | 306724 |
| Tim3 | BV605 | BioLegend | 345018 |
| TNF-α | BV605 | BioLegend | 502936 |

TABLE 3
Western Blot Antibody List

| Antibody Target | Vendor Name | Catalog Number |
|---|---|---|
| cGAS | Cell Signaling | 15102s |
| STING | Cell Signaling | 13647s |
| P-STING | Cell Signaling | 50907s |
| TBK1 | Cell Signaling | 3504s |
| P-TBK1 | Cell Signaling | 5483s |
| IRF3 | Cell Signaling | 4302s |
| P-IRF3 | Cell Signaling | 29047s |
| Aim2 | Cell Signaling | 12948s |
| Caspase 1 | Cell Signaling | 3866s |
| Cleaved Caspase 1 | Cell Signaling | 4199s |
| IL-1β | Cell Signaling | 12703s |
| Cleaved IL-1β | Cell Signaling | 83186s |
| MyD88 | Cell Signaling | 4283s |
| TRAF6 | Cell Signaling | 8028s |
| IRAK4 | Cell Signaling | 4363s |
| P-IRAK4 | Cell Signaling | 11927s |
| GAPDH | Cell Signaling | 2118s |
| GAPDH-HRP | Cell Signaling | 3683s |

TABLE 4
qPCR Primer List

| qPCR Target | Assay ID | Catalog Number |
|---|---|---|
| IFN-α | Hs00265051_s1 | 4331182 |
| IFN-β | Hs01077958_s1 | 4331182 |
| IFN-γ | Hs00989291_m1 | 4331182 |
| IL-6 | Hs00985639_m1 | 4331182 |
| RNA18S | Hs03928990_g1 | 4331182 |

TABLE 5
Exemplary cGAS-STING Pathway Inhibitors

| Name | Synonym | Structure |
|---|---|---|
| G140 | 1-(6,7-Dichloro-9-(1-methyl-1H- pyrazol-3-yl)-1,3,4,5- tetrahydro-2H- pyrido[4,3-b]indol-2-yl)-2- hydroxyethan-1-one | 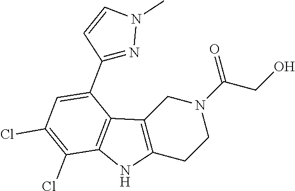 [SMILES: Cn1ccc(-c2cc(Cl)c(Cl)c3[nH]c4c(c23)CN(C(=O)CO)CC4)n1] |
| H151 | N-(4-Ethylphenyl)- N′-1H-indol-3-yl- urea | 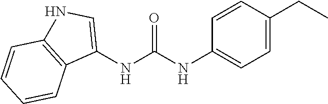 [SMILES: CCc1ccc(NC(=O)Nc2c[nH]c3ccccc23)cc1] |
| RU.521 | 3-(1-(6,7-Dichloro-1H- benzo[d]imidazol-2- yl)-5-hydroxy-3- methyl-1H-pyrazol-4- yl)isobenzofuran-1(3H)-one | 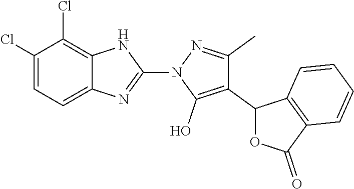 |
| BX795 | N-[3-[[5-iodo-4-[[3-[(2- thienylcarbonyl) amino]propyl]amino]- 2-pyrimidinyl]amino]phenyl]-1- Pyrrolidinecarboxamide hydrochloride | 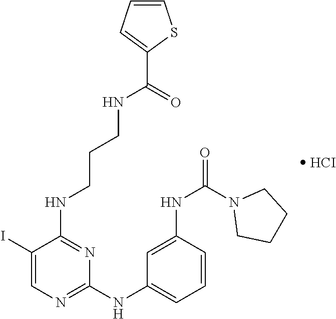 |
| Amlexanox (Aml) | 2-amino-5-oxo-7-propan-2- ylchromeno[2,3-b]pyridine-3- carboxylic acid | 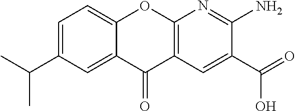 [SMILES: CC(C)c1ccc2oc3nc(N)c(C(=O)O)cc3c(=O)c2c1] |
| MRT67307 (MRT) | N-[3-[[5-cyclopropyl-2-[3- (morpholin-4- ylmethyl)anilino]pyrimidin-4- yl]amino]propyl] cyclobutanecarboxamide | 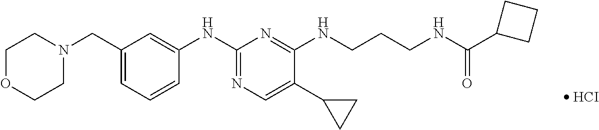 [SMILES: O=C(NCCCNc1nc(Nc2cccc(CN3CCOCC3)c2)ncc1C1CC1)C1CCC1 ; "· HCI"] |

### REFERENCES

[1] Roth T. et. al, Reprogramming human T cell function and specificity with non-viral genome targeting. Nature. 559, 405-409 (2018)
[2] Cornu, T. I., Mussolino, C. & Cathomen, T. Refining strategies to translate genome editing to the clinic. Nat. Med. 23, 415-423 (2017).
[3] Hornung, V. & Latz, E. Intracellular DNA recognition. Nat. Rev. Immunol. 10, 123-130 (2010).
[4] Luecke, S. et. al, cGAS is activated by DNA in a length-dependent manner. EMBO Rep. 18, 1707-1715 (2017)
[5] Hansen, K. et. al, Listeria monocytogenes induces IFNβ expression through an IFI16-, cGAS- and STING-dependent pathway. EMBO J. 33, 1654-1666 (2014)
[6] Rathiam V. et. al, The AIM2 inflammasome is essential for host defense against cytosolic bacteria and DNA viruses. Nature Immunology. 11, pages 395-402 (2010)
[7] Ferrer J. et. al, Dual Toll-Like Receptor Targeting Liposomal Spherical Nucleic Acids. Bioconjugate Chem. 30, 944-951 (2019)
[8] Radovic-Moreno A. et. al, Immunomodulatory spherical nucleic acids. Proc. Natl. Acad. Sci. U.S.A., 112, 3892-3897 (2015)
[9] Clark K. et. al, Use of the Pharmacological Inhibitor BX795 to Study the Regulation and Physiological Roles of TBK1 and IKB Kinase. Mechanism of Signal Trans. 284, 14136-14146 (2009)
[10] Feldman R. et. al, Novel Small Molecule Inhibitors of 3-Phosphoinositide-dependent Kinase-1. Mechanism of Signal Trans. 280, 19867-19874
[11] Steinhagen F. et. al, Suppressive oligodeoxynucleotides containing TTAGGG motifs inhibit cGAS activation in human monocytes. Eur. J. Immunol. 48, 605-61 (2018)
[12] Eichholz K. et. al, Immune-Complexed adenovirus induce AIM2-mediated pyroptosis in human dendritic cells. PLOS Pathogens. https://doi.org/10.1371/journal.ppat.1005871. (2016)
[13] Kaminski J. et al, Synthetic oligodeoxynucleotides containing suppressive TTAGGG motifs inhibit AIM2 inflammasome activation. J Immunol. 191, 3876-83 (2013)
[14] Lugli E. et. al, Superior T memory stem cell persistence supports long-lived T cell memory. J Clin Invest. 123, 594-599 (2013)
[15] Toews K. et. al, Central memory phenotype drives success of checkpoint inhibition in combination with CAR T cells. Molecular Carcinogenesis. 59, 724-735 (2020)
[16] Kaech S. et. al, Transcriptional control of effector and memory CD8+ T cell differentiation. Nature Reviews Immunology. 12, 749-761 (2012)
[17] Zhang X. et. al, Structures and Mechanisms in the cGAS-STING Innate Immunity Pathway. Immunity. 53, 43-53 (2020)
[18] Cheng W. et. al, The cGas-Sting Signaling Pathway Is Required for the Innate Immune Response Against Ectromelia Virus. Front. Immunol., 14, doi: 10.3389/fimmu.2018.01297 (2018)
[19] Wang S. et. al. Rational vaccinology with spherical nucleic acids. Proc. Natl. Acad. Sci. U.S.A., 116, 10473-10481 (2019)

1. A method of engineering a T cell, comprising contacting the T cell with a nucleic acid and one or more cyclic GMP-AMP synthase (cGAS)-stimulator of interferon gene (STING) pathway inhibitors.

2. The method of claim 1, wherein the nucleic acid comprises a donor DNA or is a double-stranded circular nucleic acid.

3. (canceled)

4. (canceled)

5. The method of claim 2, wherein the donor DNA encodes an exogenous T cell receptor (TCR)-alpha or a fragment thereof, an exogenous TCR-beta or a fragment thereof, or a combination thereof.

6. The method of claim 1, wherein the nucleic acid is associated with a delivery vehicle.

7. (canceled)

8. (canceled)

9. The method of claim 1, further comprising contacting the T cell with a gene editing reagent.

10. (canceled)

11. (canceled)

12. The method of claim 9, wherein the gene editing reagent comprises an RNA-guided nuclease.

13. The method of claim 12, wherein the RNA-guided nuclease is a CRISPR-Cas system.

14. (canceled)

15. The method of claim 13, wherein the CRISPR-Cas system comprises a Cas9, an nCas9, or a Cas-CLOVER.

16. (canceled)

17. (canceled)

18. (canceled)

19. The method of claim 9, wherein the gene editing reagent further comprises a guide RNA.

20. The method of claim 9, wherein the gene editing reagent comprises MAD7, TALEN, or ZFN.

21. (canceled)

22. (canceled)

23. (canceled)

24. The method of claim 2, wherein the donor DNA is inserted into an endogenous TCR locus.

25. (canceled)

26. The method of claim 9, wherein contacting the T cell with the gene editing reagent comprises transfecting the T cell with the gene editing reagent.

27. The method of claim 1, wherein contacting the T cell with the nucleic acid comprises transfecting the T cell with the nucleic acid.

28. The method of claim 1, wherein the one or more cGAS-STING pathway inhibitors comprises a cGAS inhibitor, a STING inhibitor, or a TANK-binding kinase 1 (TBK1) inhibitor.

29. (canceled)

30. (canceled)

31. The method of claim 1, wherein the one or more cGAS-STING pathway inhibitors is Amlexanox (Aml), MRT67307 (MRT), BX795, H151, ODN-A151 (ODN151), Ru.521, G140, or combinations thereof.

32. (canceled)

33. (canceled)

34. (canceled)

35. The method of claim 1, wherein the T cell and the nucleic acid are contacted in the presence of one or more cGAS-STING pathway inhibitors or wherein the T cell is contacted sequentially with the nucleic acid and the one or more cGAS-STING pathway inhibitors.

36. (canceled)

37. The method of claim 35, wherein the T cell is contacted with the one or more cGAS-STING pathway inhibitors prior to the nucleic acid.

38-181. (canceled)

182. A population of engineered T cells made by contacting a population of T cells with a nucleic acid and one or more cyclic GMP-AMP synthase (cGAS)-stimulator of interferon gene (STING) pathway inhibitors.

183-186. (canceled)

187. The population of engineered T cells of claim 182, wherein the nucleic acid is associated with a delivery vehicle.

188-207. (canceled)

208. A composition comprising a population of T cells, a nucleic acid, and one or more cyclic GMP-AMP synthase (cGAS)-stimulator of interferon gene (STING) pathway inhibitors.
